(19) United States
(12) Patent Application Publication
(10) Pub. No.: US 2009/0099029 A1
(43) Pub. Date: Apr. 16, 2009

(54) Methods and substrates for conducting assays

(75) Inventor: Michael Samuels, Branford, CT (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Invitrogen Corporation, Carlsbad, CA (US)

(21) Appl. No.: 11/453,256

(22) Filed: Jun. 15, 2006

Related U.S. Application Data
(60) Provisional application No. 60/690,802, filed on Jun. 15, 2005.

(51) Int. Cl. C40B 30/04; G01N 33/566

(52) U.S. Cl. 506/9; 435/7.8

(57) ABSTRACT

The present invention relates to methods of conducting kinase assays using a myelin basic protein subtrate and a tyrosine kinase. Also provided herein are compositions that include myelin basic protein and a tyrosine kinase. Illustrative embodiments of these assays are performed on a microarray. In another embodiment, provided herein is a universal substrate that includes myelin basic protein.

### CROSS-REFERENCE TO RELATED APPLICATIONS

This application claims the benefit of U.S. Provisional Application Ser. No. 60/690,802 filed Jun. 15, 2005, the disclosure of which is hereby incorporated by reference in its entirety.

### BACKGROUND OF THE INVENTION

1. Field of the Invention

The present invention relates to methods of conducting assays for kinase activity on microarrays useful for the large-scale study of protein function, screening assays, and high-throughput analysis of kinase reactions. The invention relates to methods of using protein chips to assay the presence, amount, activity and/or function of kinases present in a protein sample on a protein chip. In particular, the methods of the invention relate to conducting enzymatic assays using a microarray wherein a kinase and a substrate are immobilized on the surface of a solid support and wherein the kinase and the substrate are in proximity to each other sufficient for the occurrence of an enzymatic reaction between the substrate and the kinase. The invention also relates to microarrays that have a kinase and a substrate immobilized on their surface wherein the kinase and the substrate are in proximity to each other sufficient for the occurrence of an enzymatic reaction between the kinase and the substrate.

2. Background Art

A daunting task in the post-genome sequencing era is to understand the functions, modifications, and regulation of every protein encoded by a genome (Fields et al., 1999, Proc Natl Acad. Sci. 96:8825; Goffeau et al., 1996, Science 274:563). Currently, much effort is devoted toward studying gene, and hence protein, function by analyzing mRNA expression profiles, gene disruption phenotypes, two-hybrid interactions, and protein subcellular localization (Ross-Macdonald et al., 1999, Nature 402:413; DeRisi et al., 1997, Science 278:680; Winzeler et al., 1999, Science 285:901; Uetz et al., 2000, Nature 403:623; Ito et al., 2000, Proc. Natl. Acad. Sci. U.S.A. 97:1143). Important advances in this effort have been possible, in part, by the ability to analyze thousands of gene sequences in a single experiment using gene chip technology. Although these studies are useful, transcriptional profiles do not necessarily correlate well with cellular protein levels or protein activities. Thus, the analysis of biochemical activities can provide information about protein function that complements genomic analyses to provide a more complete picture of the workings of a cell (Zhu et al., 2001, Curr. Opin. Chem. Biol. 5:40; Martzen, et al., 1999, Science 286:1153; Zhu et al., 2000, Nat. Genet. 26:283; MacBeath, 2000, Science 289:1760; Caveman, 2000, J. Cell Sci. 113:3543).

Currently, biochemical analyses of protein function are performed by individual investigators studying a single protein at a time. This is a very time-consuming process since it can take years to purify and identify a protein based on its biochemical activity. The availability of an entire genome sequence makes it possible to perform biochemical assays on every protein encoded by the genome. Based on sequence comparison, genes encoding for proteins with a particular enzymatic activity can be identified. However, a detailed analysis of an individual proteins' biochemical properties, such as, substrate specificity, kinetic profile and sensitivities to inhibitors, is a time-consuming process. Thus, high-throughput ways of analyzing the biochemical activities of proteins are required.

It would be useful to analyze hundreds or thousands of protein samples using a single protein chip. Such approaches lend themselves well to high throughput experiments in which large amounts of data can be generated and analyzed. Microtiter plates containing 96 or 384 wells have been known in the field for many years. However, the size (at least 12.8 cm×8.6 cm) of these plates makes them unsuitable for the large-scale analysis of proteins.

Recently devised methods for expressing large numbers of proteins with potential utility for biochemical genomics in the budding yeast Saccharomyces cerevisiae have been developed. ORFs have been cloned into an expression vector that uses the GAL promoter and fuses the protein to a polyhistidine (e.g., HISX6) label. This method has thus far been used to prepare and confirm expression of about 2000 yeast protein fusions (Heyman et al., 1999, “Genome-scale cloning and expression of individual open reading frames using topoisomerase I-mediated ligation,” Genome Res. 9:383-392). Using a recombination strategy, about 85% of the yeast ORFs have been cloned in frame with a GST coding region in a vector that contains the CUP1 promoter (inducible by copper), thus producing GST fusion proteins (Martzen et al., 1999, “A biochemical genomics approach for identifying genes by the activity of their products,” Science 286:1153-1155). Martzen et al. used a pooling strategy to screen the collection of fusion proteins for several biochemical activities (e.g., phosphodiesterase and Appr-1-P-processing activities) and identified the relevant genes encoding these activities.

Several groups have recently described microarray formats for the screening of protein activities (Zhu et al., 2000, Nat. Genet. 26:283; MacBeath et al., 2000, Science 289:1763; Arenkov et al, 2000, Anal. Biochem 278:123). In addition, a collection of overexpression clones of yeast proteins have been prepared and screened for biochemical activities (Martzen et al., 1999, Science 286: 1153).

Photolithographic techniques have been applied to making a variety of arrays, from oligonucleotide arrays on flat surfaces (Pease et al., 1994, “Light-generated oligonucleotide arrays for rapid DNA sequence analysis,” PNAS 91:5022-5026) to arrays of channels (U.S. Pat. No. 5,843,767) to arrays of wells connected by channels (Cohen et al., 1999, “A microchip-based enzyme assay for protein kinase A,” Anal Biochem. 273:89-97). Furthermore, microfabrication and microlithography techniques are well known in the semiconductor fabrication area. See, e.g., Moreau, Semiconductor Lithography: Principals, Practices and Materials, Plenum Press, 1988.

Screening a large number of proteins or even an entire proteome would entail the systematic probing of biochemical activities of proteins that are produced in a high throughput fashion, and analyzing the functions of hundreds or thousands of proteins samples in parallel (Zhu et al., 2000, Nat. Genet. 26:283; MacBeath et al., 2000, Science 289:1763; Arenkov et al, 2000, Anal. Biochem 278:123; International Patent Application publication WO 01/83827 and WO 02/092118). In vitro assays have previously been conducted using random expression libraries or pooling strategies, both of which have shortcomings (Martzen et al., 1999, Science 286:1153; Bussow et al., 2000, Genomics 65:1). Specifically, random expression libraries are tedious to screen, and contain clones that are often not full-length. Another recent approach has been to generate defined arrays and screen the array using a pooling strategy (Martzen et al. 1999, Science 286:1153). The pooling strategy obscures the actual number of proteins screened, however, and the strategy is cumbersome when large numbers of positives are identified.

Kinases are proteins known to play important roles in many of the functions of all eukaryotic cells, including mammalian cells. Therefore, they are believed to be involved in disease formation and progression, and can be the target of drug treatment. Accordingly, considerable work continues on identifying new methods for identifying drug candidates that affect the activity of particular kinases. Especially valuable new methods include those that can be performed in a high-throughput manner, for a large number of kinases and a large number of drug candidates.

Citation or identification of any reference in this application shall not be considered as admission that such reference is available as prior art to the present invention.

### SUMMARY OF THE INVENTION

The present invention is based in part on the discovery that myelin basic protein (MBP) can serve as a substrate for numerous tyrosine kinases. Furthermore, the present invention is based on the discovery that for kinase assays that utilize immobilized MBP, such as those utilize a substrate coated with MBP, non-phosphorylated MBP, such as that produced in a prokaryotic cell, is a preferred substrate. Finally, the present invention in certain illustrative embodiments, utilizes MBP or a fragment or derivative thereof, in a fusion protein that includes additional kinase substrates.

The present invention provides methods, kits, and microarrays for kinase assays that utilize immobilized MBP. The present invention also provides methods, kits, and microarrays for identifying modulators of kinase activities using immobilized MBP.

An aspect of the present invention are methods for detecting phosphorylation of myelin basic protein (MBP) by a kinase, wherein the method includes: (a) incubating a tyrosine kinase and MBP, or a fragment or derivative thereof comprising at least 15 contiguous amino acids of MBP, or one or more conservative substitutions thereof, and comprising at least one phosphorylation site of MBP within the at least 15 contiguous amino acids, under conditions allowing for phosphorylation of the MBP or fragment or derivative thereof by the tyrosine kinase; and, (b) detecting phosphorylation of the MBP, or the fragment or derivative thereof. In an embodiment of this aspect, the incubating step is done in the presence of a test molecule. In further or alternative embodiments, the detecting step comprises detecting a decrease in the phosphorylation in the presence of the test molecule, thereby identifying the test molecule as an inhibitor of the kinase, while in still further or alternative embodiments. the detecting step comprises detecting an increase in the phosphorylation in the presence of the test molecule, thereby identifying the test molecule as an activator of the kinase. In further or alternative embodiments, step (b) of such methods includes detecting phosphorylated tyrosines on the myelin basic protein or the fragment or derivative thereof. In still further or alternative embodiments, the determining step includes contacting myelin basic protein, or a fragment or derivative thereof, with a binding partner that selectively binds to the phosphorylated or non-phosphorylated form of MBP or a fragment thereof. In further or alternative embodiments, incubating step is done in the presence of a test molecule so as to determine whether the test molecule modulates the reaction. In even further or alternative embodiments, the determining step includes detecting whether a change in the phosphorylation rate on occurs, or determining whether the phosphorylation occurs at all, in the presence of the test molecule relative to the amount of the reaction in the absence of the test molecule. In still further or alternative embodiments, a test molecule can be identified as an inhibitor of the phosphorylation of MBP, or the fragment or derivative thereof, by the kinase using the method.

In further or alternative embodiments the tyrosine kinase used in such methods is a tyrosine kinase of Table 2 or Table 6. In further or alternative embodiments, such the tyrosine kinase used in such methods is selected from CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and YES1. In further or alternative embodiments, such the tyrosine kinase used in such methods is selected from CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC. In further or alternative embodiments, the tyrosine kinase is selected from two or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC, while in further or alternative embodiments, the tyrosine kinase is selected from five or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC. In still further or alternative embodiments, the tyrosine kinase is selected from ten or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

In other embodiments of such methods, the MBP or the fragment or derivative thereof, is MBP or a fragment thereof comprising at least 15 contiguous amino acids of MBP. In further or alternative embodiments, the MBP or the fragment or derivative thereof, is full length MBP. In further or alternative embodiments, the MBP or the fragment or derivative thereof, is full length human MBP or a fragment thereof comprising at least 15 contiguous amino acids of human MBP. In further or alternative embodiments, the MBP or the fragment or derivative thereof, is full length bovine MBP or a fragment thereof comprising at least 15 contiguous amino acids of bovine MBP. In further or alternative embodiments, the MBP or fragment or derivative thereof, at the start of the incubating, is not phosphorylated. In further or alternative embodiments, such methods further include isolating the MBP or the fragment or derivative thereof from a prokaryotic host cell.

In further or alternative embodiments of such methods, at least one of the tyrosine kinase and the MBP or the fragment or derivative thereof, are immobilized on the surface of a solid support, while in further or alternative embodiments, both the tyrosine kinase and the MBP or the fragment or derivative thereof, are immobilized on the surface of a solid support. In further or alternative embodiments, the MBP or the fragment or derivative thereof, is coated onto the surface of the solid support and the kinase is deposited onto the surface of the solid support. In further or alternative embodiments, the kinase is coated onto the surface of the solid support and the MBP or the fragment or derivative thereof is deposited onto the surface of the solid support. In further or alternative embodiments, a kinase substrate other than MBP or a fragment or derivative thereof, is coated onto the surface of the solid support along with MBP or a fragment or derivative thereof. In further or alternative embodiments, a plurality of kinases are immobilized on the solid support, wherein at least one of the plurality of kinases is other than a tyrosine kinase. In further or alternative embodiments, the plurality of different kinases consists of between two different kinases and 10,000 different kinases. In further or alternative embodiments, the plurality of different kinases consists of between two and 1000 different mammalian kinases. In further or alternative embodiments, the plurality of different kinases consists of between two and 1000 different human kinases. In further or alternative embodiments, the plurality of different kinases comprises a tyrosine kinase and a serine/threonine kinase. In still further or alternative embodiments, the detecting includes detecting phosphorylation of MBP, or the fragment or derivative thereof, by the tyrosine kinase and/or by the serine/threonine kinase, wherein both the tyrosine kinase and the serine/threonine kinase phosphorylate MBP, or the fragment or derivative thereof. In further or alternative embodiments, the kinase and the MBP or the fragment or derivative thereof, are deposited using a microarray robot, pins, or a piezo electric field. In further or alternative embodiments, the solid support comprises at least two wells and wherein each well comprises the substrate and the kinase.

In further or alternative embodiments of such methods, a plurality of different substrates are immobilized on the solid support. In further or alternative embodiments, at least one of the plurality of different substrates is other than MBP or a fragment or derivative thereof. In further or alternative embodiments, the plurality of different substrates consists of between one and ten different substrates.

In further or alternative embodiments of such methods the tyrosine kinase is a receptor tyrosine kinase. In further or alternative embodiments, the tyrosine kinase is a cytoplasmic tyrosine kinase.

In further or alternative embodiments of such methods, the MBP or the fragment or derivative thereof, is a first amino acid sequence of a recombinant fusion protein further comprising a second amino acid sequence comprising a kinase substrate other than MBP or a fragment or derivative thereof. In further or alternative embodiments the second amino acid sequence is a substrate for a kinase that does not phosphorylate MBP. In further or alternative embodiments, the recombinant fusion protein comprises additional amino acid sequences that are kinase substrate such that the recombinant fusion protein is phosphorylated by at least 100 kinases.

Another aspect of the invention described herein are recombinant substrates having a first amino acid sequence corresponding to at least 15 contiguous amino acids of myelin basic protein and a second amino acid sequence different from the first amino acid sequence, wherein either or both of the first and second amino acid sequences have the ability to serve as a substrate for a kinase. In an embodiment of such substrates the 15 contiguous amino acids of myelin basic protein comprise a tyrosine residue. In further or alternative embodiments, the first amino acid sequence is full-length myelin basic protein. In further or alternative embodiments, the second amino acid sequence is flanked by a sequence corresponding to at least a portion of myelin basic protein. In further or alternative embodiments, the C-terminus of the second amino acid sequence is adjacent to the N-terminus of the first amino acid sequence. In further or alternative embodiments, the N-terminus of the second amino acid sequence is adjacent to the C-terminus of the first amino acid sequence. In further or alternative embodiments, the second amino acid is a substrate for a kinase that does not phosphorylate MBP. In further or alternative embodiments, the first amino acid sequence is not phosphorylated. In further or alternative embodiments, the second amino acid sequence is not phosphorylated. In further or alternative embodiments, neither the first amino acid sequence nor the second amino acid sequence are phosphorylated. In further or alternative embodiments, the substrate is phosphorylated on at least one serine, threonine or tyrosine residue. In further or alternative embodiments, the substrate is phosphorylated on at least one tyrosine residue. In further or alternative embodiments, the at least 15 contiguous amino acids of MBP are phosphorylated on at least one tyrosine residue. In further or alternative embodiments, the substrate is produced in a prokaryotic host cell. In further or alternative embodiments, the substrate is deposited on a solid support. In further or alternative embodiments, the solid support comprises a kinase immobilized on the surface of the solid support. In further or alternative embodiments, the solid support comprises an array of a plurality of different kinases immobilized on the surface of the solid support.

Another aspect of the invention described herein are methods for detecting phosphorylation of a recombinant substrate, the method which include: (a) incubating a kinase and the recombinant substrate under conditions allowing for a reaction between the kinase and the recombinant substrate, wherein the recombinant substrate comprises a first amino acid sequence corresponding to at least 15 contiguous amino acids of myelin basic protein and a second amino acid sequence different from the first amino acid sequence, wherein either or both of the first and second amino acid sequences have the ability to serve as a substrate for a kinase; and, (b) detecting phosphorylation of the recombinant substrate. In an embodiment of such methods, the incubating step is done in the presence of a test molecule. In further or alternative embodiments, the detecting step comprises detecting a decrease in the phosphorylation in the presence of the test molecule, thereby identifying the test molecule as an inhibitor of the kinase. In further or alternative embodiments, the detecting step comprises detecting an increase in the phosphorylation in the presence of the test molecule, thereby identifying the test molecule as an activator of the kinase. In further or alternative embodiments, the kinase is a tyrosine kinase. In further or alternative embodiments, the tyrosine kinase is a tyrosine kinase of Table 2 or Table 6. In further or alternative embodiments the tyrosine kinase is selected from CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and YES1. In further or alternative embodiments, the tyrosine kinase is selected from CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC. In further or alternative embodiments, the method further includes incubating a second kinase with the recombinant substrate. In further or alternative embodiments, the method further includes incubating a plurality of kinases with the recombinant substrate, wherein the plurality of kinases comprise a tyrosine kinase and a serine/threonine kinase. In further or alternative embodiments, both the kinase and the recombinant substrate, are immobilized on the surface of a solid support. In further or alternative embodiments the recombint substrate is coated onto the surface of the solid support and the kinase is deposited onto the substrate. In further or alternative embodiments, a plurality of kinases are immobilized on the solid support, wherein at least one of the plurality of kinases is other than a tyrosine kinase. In further or alternative embodiments, the plurality of different kinases comprises a tyrosine kinase and a serine/threonine kinase. In further or alternative embodiments, the detecting includes detecting phosphorylation of MBP, or the fragment or derivative thereof, by the tyrosine kinase and by the serine/threonine kinase.

Another aspect of the invention described herein are kits which include a recombinant substrate comprising a first amino acid sequence corresponding to at least 15 contiguous amino acids of myelin basic protein and a second amino acid sequence different from the first amino acid sequence, wherein either or both of the first and second amino acid sequences have the ability to serve as a substrate for a kinase, and a detectable agent that differentially binds to a phosphorylated reside of the recombinant substrate. In an embodiment of such kits, the kits also include a kinase capable of phosphorylating the recombinant substrate. In further or alternative embodiments, the detectable agent has the ability to bind to phosphosphorylated amino acid residues. In further or alternative embodiments, the detectable agent is a dye that binds to phosphotyrosine residues. In further or alternative embodiments, the kinase comprises a tyrosine kinase of Table 2 or Table 6. In further or alternative embodiments, the tyrosine kinase is selected from CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and YES1. In further or alternative embodiments, the tyrosine kinase is selected from CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

Another aspect of the invention described herein are kits which include a non-phosphorylated myelin basic protein (MBP) and a tyrosine kinase capable of phosphorylating MBP. In an embodiment of such kits, the kits also include a detectable agent having the ability to bind to phosphosphorylated amino acid residues. In further or alternative embodiments, the detectable agent is a dye that binds to phosphotyrosine residues. In further or alternative embodiments, the tyrosine kinases comprises a tyrosine kinase of Table 2 or Table 6. In further or alternative embodiments, the tyrosine kinase is selected from CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and YES1. In further or alternative embodiments, the tyrosine kinase is selected from CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

### DEFINITIONS AND ABBREVIATIONS

As used in this application, “protein” refers to a peptide or polypeptide. Proteins can be prepared from recombinant overexpression in an organism, preferably bacteria, yeast, insect cells or mammalian cells, or produced via fragmentation of larger proteins, or chemically synthesized.

As used in this application, “enzyme” refers to any protein with a catalytic activity.

As used in this application, “functional domain” is a domain of a protein which is necessary and sufficient to give a desired functional activity. Examples of functional domains include, inter alia, domains which exhibit an enzymatic activity such as oxidoreductase, transferase, hydrolase, lyase, isomerase, or ligase activity. In more specific embodiments, a functional domain exhibits kinase, protease, phosphatase, glycosidase, or acetylase activity. Other examples of functional domains include those domains which exhibit binding activity towards DNA, RNA, protein, hormone, ligand or antigen.

Each protein or substrate of an enzymatic reaction on a chip is preferably located at a known, predetermined position on the solid support such that the identity of each protein or probe can be determined from its position on the solid support. Further, the proteins and probes form a positionally addressable array on a solid support.

As used herein, the term “purified” refers to a molecule, a substrate or a protein that is substantially free of different molecules of the same type, substrates of the same type, or proteins, respectively, that are associated with it in its original state (from which it is purified). Preferably, a molecule is at least 50%, 60%, 70%, 80%, 90%, 95%, 98%, 99%, 99.5%, 99.8%, 99.9%, 99.98%, 99,998%, 99,9998%, 99,99998% or at least 99,999998% free of such different molecules, wherein, if the molecule is in solution, the solvent is not a different molecule. Preferably, a substrate is at least 50%, 60%, 70%, 80%, 90%, 95%, 98%, 99%, 99.5%, 99.8%, 99.9%, 99.98%, 99,998%, 99, 9998%, 99,99998% or at least 99,999998% free of such different substrates. Preferably, a protein is at least 50%, 60%, 70%, 80%, 90%, 95%, 98%, 99%, 99.5%, 99.8%, 99.9%, 99.98%, 99,998%, 99, 9998%, 99,99998% or at least 99,999998% free of such different proteins.

### ABBREVIATIONS

Abbreviation

RIE Reactive Ion Etching

GST glutathione-S-transferase

GPTS 3-glycidooxypropyltrimethoxysilane

ORF Open reading frame

FRET Fluorescence Resonance Energy Transfer

### BRIEF DESCRIPTION OF THE DRAWINGS/FIGURES

FIG. 1 illustrates the detection of kinase activity using ProQ Diamond staining and antibody-based detection of His-tagged kinases using a microarray-based screening assay.

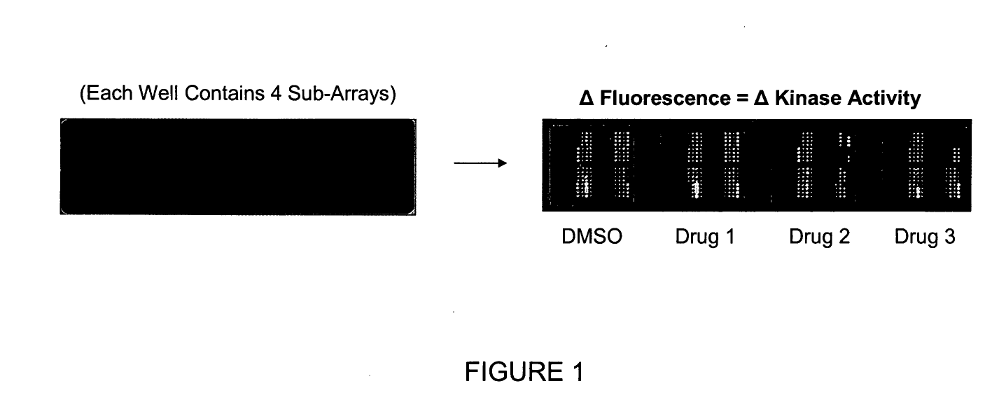

FIG. 2 illustrates the effects of various reaction times on kinase activity. Shown are images of the screening assay where kinase activity was detected using the ProQ Diamond Stain (left two panels) and kinase presence detected using an antibody to the His 6 epitope-tag present on the kinases (right two panels). For each detection system, only the right panel is coated with MBP on the slide (i.e., the left panel is a negative control). Equal amounts of kinases are present on the two slides. The antibody staining demonstrates that kinases are equally present on both substrate-coated and non-coated slides (i.e. this is a control). The ProQ stain demonstrates that fluorescence is only detected on the substrate-coated slide (and is localized to where the kinases have been spotted). Thus, fluorescence is dependent on the presence of both kinase and substrate.

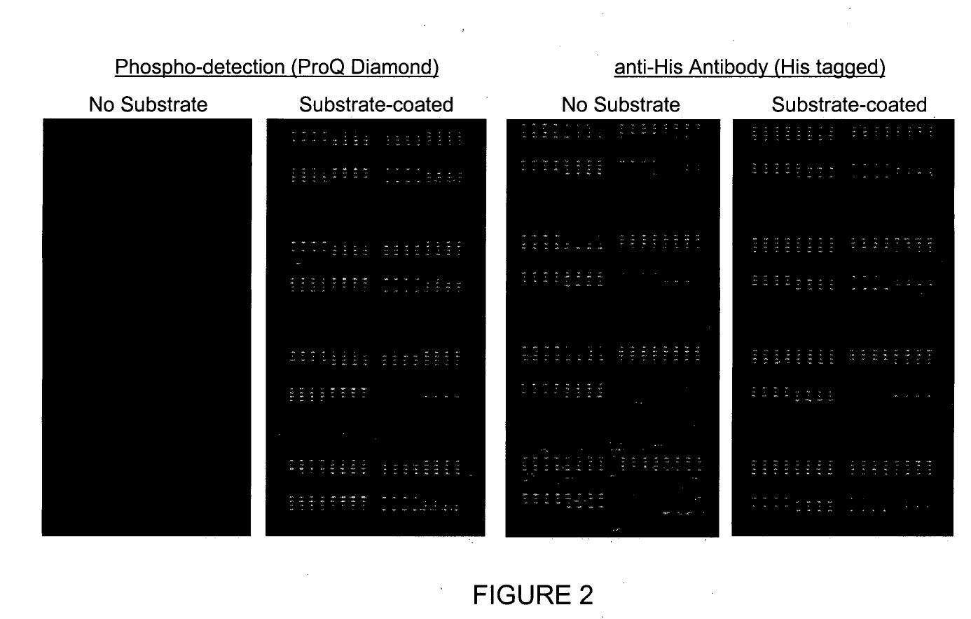

FIG. 3 is a schematic of the four-well microKIP Assay Format. The images shown are of a series of MBP-coated slides from the same print run after different amounts of time (7.5 minutes, 15 minutes, 30 minutes, 60 minutes, and 90 minutes) in reaction buffer and detected using ProQ Diamond Stain. The colored circles highlight the change in kinase activity over time for two kinases (red and green), or show no change for the control protein (BSA with a phosho-tyrosine residue attached). This figure illustrates that the kinases are acting in a catalytic manner to phosphorylate the MBP-coated slide.

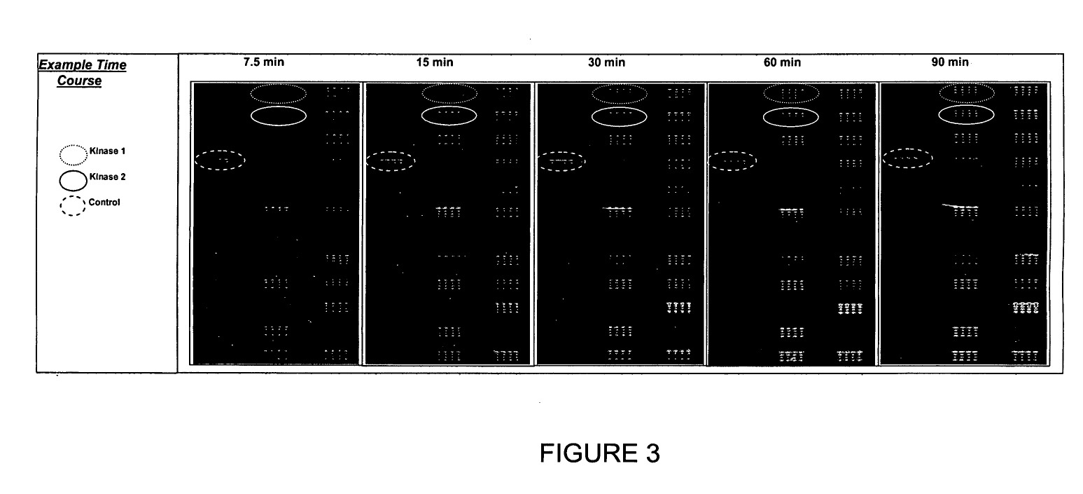

FIG. 4 is a schematic of a four-well slide, each of the four wells containing four sub-arrays, for assaying the effects of various compounds on kinase activity against a substrate. On the left a single sub-array is shown, with the density of kinases allowed when using either an 8×8 subarray, or a 16×16 subarray. This allows 256 kinases to be assayed (in quadruplicate) on a single well of the microarray slide. The middle panel shows the layout of the slide, with four clear areas (each capable of fitting four subarrays) surrounded by a hydrophobic coating, allowing for one slide to have four “reaction chambers” each containing identical kinases. The panel on the right is one example of the expected use of the array, with one well being a control (DMSO) and the other well's having different chemical compounds present during the reaction. Reduction of the fluorescent signal present in the control well by the compound treatment would identify specific kinase inhibition by the compound.

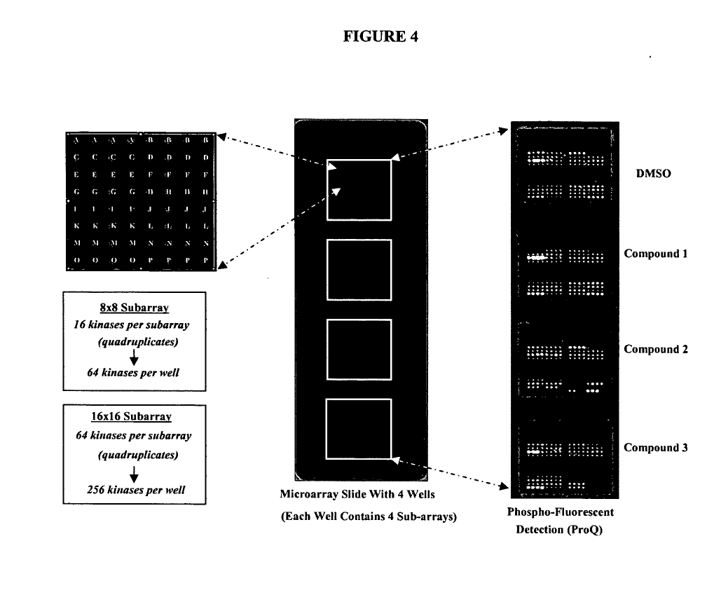

FIG. 5 illustrates the following sequences: (A) Human MBP cDNA sequence (GenBank BC080654) (SEQ ID NO:2). (B) Human MBP amino acid sequence (SEQ ID NO:1), and (C) General structure of a universal substrate (SEQ ID NO:24).

### DETAILED DESCRIPTION OF THE INVENTION

Methods of conducting assays for enzymatic activity on microarrays have been described in U.S. Patent Publication No. 2004-0248323, the disclosure of which is hereby incorporated by reference in its entirety. The invention is directed to methods of conducting assays for kinase enzymatic activity on protein microarrays (also referred to herein as protein chips). In the methods of the invention, a substrate and a kinase, both immobilized on the surface of the microarray, are in proximity with each other sufficient for the occurrence of an enzymatic reaction between the substrate and the kinase. The present invention also provides methods of using protein chips to assay the presence, amount, functionality, activity and sensitivity to modulators of kinases. The invention further provides microarrays containing a substrate and a kinase, both immobilized on the surface of the microarray, wherein the substrate and the kinase are in proximity with each other sufficient for the occurrence of an enzymatic reaction between the substrate and the kinase. The use of such microarrays includes, but is not limited to, determining whether the substrate is a substrate and/or if the kinase is an enzyme that acts on the substrate, determining kinase enzymatic activity, and to identify modulators of the kinase enzymatic reaction.

In certain embodiments, the methods of the invention can be used to identify kinases that catalyze a specific reaction. In certain embodiments, the methods of the invention can be used to identify kinases that use a specific substrate. In these embodiments, one or more kinases that are candidates for the enzyme that catalyzes the reaction of interest are immobilized on a protein chip for use with the invention.

In certain embodiments, the methods of the invention can be used to identify substrates of a kinase of interest. In certain embodiments, the methods of the invention can be used to identify substrates that are used by kinases having a specific catalytic activity. In certain embodiments, the methods of the invention can be used to identify substrates that are used by a class of kinases or by a specific kinase of interest. In these embodiments, one or more substrates that are candidates for substrates of the class of kinases or for the kinase of interest are immobilized on the surface of a solid support.

In certain embodiments of the invention, the substrate immobilized on the solid support is a reactant (i.e., a substrate) of the kinase immobilized on the solid support. In even more specific embodiments, the enzymatic reaction that occurs between the kinase and the substrate during the incubation step is a reaction that involves the substrate as a reactant (e.g. substrate) and the kinase as an enzymatic catalyst.

In additional embodiments, a plurality of substrates is immobilized on a solid support that includes at least one substrate for more than one different subclass of kinases. Accordingly, methods provided herein allow the screening of test molecules in a single reaction, for their ability to modulate enzymatic reactions of many different subclasses kinases. For example, the plurality of substrates can include substrates of many or all known subclasses of kinases in a species of organisms. In these examples, kinases immobilized on the solid support along with the plurality of substrates can include at least one representative kinase from each subclass for which a corresponding substrate is immobilized. In an illustrative example, the substrate is a mixture of Myelin Basic Protein (MBP), histone and casein. In another illustrative example, the substrate is a mixture of Myelin Basic Protein (MBP), histone, casein and/or poly(Glu4Tyr).

In certain embodiments, the methods of the invention can be used to identify modulators of kinase activity. In such screening assays, a molecule that increases or decreases the kinase activity being assayed can be identified. In certain embodiments, molecules that alter the substrate specificity of a kinase can be identified. In other embodiments, the kinetic properties of an inhibitor, an activator or a molecule that alters the substrate specificity of a kinase can be assessed.

In certain embodiments, a method of the invention for assaying a kinase reaction comprises the following steps: (a) incubating at least one kinase and at least one substrate under conditions conducive to the occurrence of an enzymatic reaction between the kinase and the substrate, wherein (i) the kinase and the substrate are immobilized on the surface of a solid support; (ii) the kinase and the substrate are in proximity sufficient for the occurrence of said enzymatic reaction; and (iii) the kinase and the substrate are not identical; and (b) determining whether a kinase reaction occurs.

In certain embodiments, a method of the invention comprises the steps of (i) immobilizing a substrate on a solid support; (ii) depositing a plurality of different kinases on the solid support such that a substrate and a kinase are in proximity sufficient for the occurrence of an enzymatic reaction between the substrate and the kinases; and (iii) detecting the occurrence of the enzymatic reaction. In certain embodiments, a method of the invention comprises the steps of (i) immobilizing a kinase on a solid support; (ii) depositing a plurality of different substrates on the solid support such that a substrate and a kinase are in proximity sufficient for the occurrence of an enzymatic reaction; and (iii) detecting the occurrence of the enzymatic reaction between the substrate and the kinase. In certain, more specific embodiments, the occurrence of the enzymatic reaction is visualized and/or quantified by a detectable signal.

In certain embodiments, a plurality of kinases is deposited on the surface of the solid support in a positionally addressable fashion such that the identity of a kinase that is located at a specific position of the array can be easily determined. In certain embodiments, a plurality of substrates is deposited on the surface of the solid support in a positionally addressable fashion such that the identity of a substrate that is located at a particular position of the array can be easily determined. A positionally addressable array provides a configuration such that each substrate and/or kinase of interest is located at a known, predetermined position on the solid support such that the identity of each substrate and/or kinase can be determined from its position on the array.

In certain aspects of the invention, a plurality of kinases and a plurality of substrates are deposited on the surface of a solid support. In these aspects, by way of example only, a plurality of substrates and a plurality of kinases can be immobilized in specific regions such that a kinase is immobilized in a region that is identical to, or overlaps with, a region that includes a specific substrate for the immobilized kinase. The regions of kinases and substrates can be obtained, by way of example only, by printing the enzymes and substrates using a microarray printer.

In certain embodiments, the surface of the solid support is coated with a substrate of a kinase reaction and the plurality of different kinases is deposited on top of the substrate coating. In certain, more specific embodiments, each kinase of the plurality of kinases is immobilized at a different position of the surface of the solid support. In other embodiments, the surface of the solid support is coated with a plurality of different substrates and the plurality of different kinases is deposited on top of each substrate. In certain, more specific embodiments, the different substrates are coated on the surface as a mixture. In other embodiments, each substrate of the plurality of substrates is coated in a different area of the solid support. In other embodiments, a substrate is deposited on the surface of the solid support and the plurality of different kinases is deposited on top of the substrate. In certain embodiments, a plurality of different substrates is deposited on the surface of the solid support and the plurality of different kinases is deposited on top of the substrates. In a specific embodiment, all possible substrate-kinase combinations of a set of kinases of interest and a set of substrates of interest are present on a single microarray. In certain, more specific, embodiments, the substrates and/or the kinases are purified.

Coating of a feature (i.e., a substrate or a kinase) typically involves a region of a solid support, i.e., the feature is contiguously immobilized on the surface of the solid support within the region such that one or more additional features (i.e., substrate or protein) can be immobilized within the region, e.g., deposition by printing. In more specific embodiments, a coated region is defined by walls or boundaries that contain a liquid applied to the surface of the solid support, and by a region of the surface within the walls or boundaries that is functionalized for immobilization of the kinase or substrate. In certain embodiments, the region covers the entire surface of the solid support. In other embodiments, multiple regions can be coated on the surface of a solid support by separating the surface of the solid support into distinct liquid regions using walls or boundaries, such as walls of wells placed on top of the surface or patterning of a hydrophobic layer to define regions for immobilization.

Printing on the other hand, typically involves applying a volume of liquid that is sufficiently small such that it does not cover the entire surface of a solid support or does not cover the entire surface of a region of a solid support that is defined by a liquid boundary, such as defined by a well or hydrophobic boundary. In this manner, a microarray containing spots of the deposited feature is obtained. Therefore, where a kinase is coated onto a surface of a solid support and the substrate is deposited onto the surface of the solid support, the coated kinasen will typically cover a larger area than the deposited substrate. Conversely, where a substrate is coated onto a surface of a solid support and the kinase is deposited onto the surface of the solid support, the coated substrate will typically cover a larger area than the deposited kinase. Illustrative methods for printing/depositing and coating onto microarrays are provided herein. Numerous methods for printing/depositing and coating onto solid supports are known in the art.

In certain embodiments, the different kinases of the plurality of different kinases are immobilized at different positions on the surface of the solid support. In certain, more specific embodiments, at least one kinase of the plurality of different kinases is immobilized at least 2, 3, 4, 5, 6, 7, 8, 9, 10, 12, 15, 20, 25, 50, or at least 100 different locations on the surface of the solid support. In a preferred embodiment, each kinase is immobilized at least 4 different positions on the surface of the solid support.

In certain embodiments, the different substrates of the plurality of different substrates are immobilized at different positions on the surface of the solid support. In certain, more specific embodiments, at least one substrate of the plurality of different substrates is immobilized at least 2, 3, 4, 5, 6, 7, 8, 9, 10, 12, 15, 20, 25, 50, or at least 100 different locations on the surface of the solid support. In a preferred embodiment, each substrate is immobilized at least 4 different positions on the surface of the solid support.

In certain embodiments, the surface of the solid support is coated with kinase and a plurality of different substrates is deposited on top of the kinase coating. In certain, more specific embodiments, each substrate of the plurality of substrates is immobilized at a different position of the surface of the solid support. In other embodiments, the surface of the solid support is coated with a plurality of different kinases and a plurality of different substrates is deposited on top of each different kinase. In certain, more specific embodiments, the different kinases are immobilized on the surface of the solid support as a mixture. In other, more specific embodiments, the different kinases are immobilized in different regions of the surface of the solid support. In other embodiments, a kinase is deposited on the surface of the solid support and a plurality of different substrates is deposited on top of the kinase. In certain embodiments, a plurality of different kinases is deposited on the surface of the solid support and a plurality of different substrates is deposited on top of the kinases. In a specific embodiment, all possible kinase-substrate combinations are present on a single microarray. In certain, more specific, embodiments, the substrates and/or the kinases are purified.

In certain embodiments, the plurality of kinases includes different kinases that are derived from the same source or the same species, such as, by way of example only, human, yeast, mouse, rat, bacteria, and C. elegans. In certain embodiments, the plurality of kinases consists of different kinases that are known to have a specific enzymatic activity. In certain other embodiments, the plurality of kinases on the microarray includes different kinases derived from different sources or from different species and where the kinases may have different or unknown enzymatic activity.

In certain embodiments, a substrate and/or a kinase are directly immobilized on a glass surface. In certain embodiments, the surface of the solid support is treated with an aldehyde before a substrate and/or kinase is immobilized on the surface. Methods for immobilizing substrates and kinases on a solid support are described in more detail herein.

In certain embodiments, the substrate includes a cofactor, as described further herein, or a candidate cofactor. Accordingly, in certain embodiments, a kinase is immobilized on the surface of a solid support and a substrate and a cofactor or a candidate cofactor are immobilized on the surface of a solid support such that the kinase and the cofactor can physically interact with each other under suitable conditions (i.e., suitable buffer and temperature). Reaction buffer containing a substrate or a candidate substrate is then added to provide conditions suitable for the occurrence of a kinase reaction. In certain embodiments, multiple different kinases and multiple different cofactors are immobilized on the surface of a solid support such that different kinase-cofactor combinations are immobilized in different locations of the solid support. In an illustrative, non-limiting, example, two different cofactors are each immobilized in a different region of the surface of the solid support. Five different kinases are each immobilized in a different location within the each region such that ten different kinase-cofactor combinations are located on the surface of the solid support and each combination is positionally addressable. Subsequently, reaction buffer with a substrate of the enzymes is added to determine which of the kinase-cofactor combinations provides the highest enzymatic activity.

In certain embodiments, if kinases are to be identified, a plurality of different kinases is deposited on the surface of the solid support together with a substrate that is known to be used in a specific kinase reaction, wherein each kinase is immobilized at a different position of the microarray. In other embodiments, if a kinase substrate is to be identified, a plurality of different substrates (i.e., candidate substrates) is deposited on the surface of the solid support together with a specific kinase, wherein each substrate is immobilized at a different position of the microarray. Any method known to the skilled artisan can be used to visualize and to quantify the kinase reaction. More detailed description of kinase reactions and their visualization are described further herein.

In certain embodiments, a substrate and a kinase are immobilized on the surface of a solid support within a well. In certain embodiments, each well on the solid support contains at least one kinase and at least one substrate such that kinase and substrate are in proximity sufficient for the occurrence of an enzymatic reaction between the substrate and the kinase. In other embodiments, a plurality of different kinases or different substrates is deposited onto the surface of the solid support such that each well harbors a plurality of different kinases or substrates. In certain, more specific embodiments, the plurality of kinases or substrates is organized in a positionally addressable array on the surface within a well. The solid support, e.g., a slide, can have at least 1, 2, 3, 4, 5, 6, 7, 8, 9, 10, 15, 20, 25, 50, 100, 1,000 or at least 10,000 wells. The performance of the kinase reaction on a solid support with wells has the advantage that different reaction solutions can be added at the same time onto one solid support (e.g., on one slide).

In certain specific embodiments, the bottom surface of a well is coated with a substrate of a kinase reaction, wherein the substrate is immobilized on the surface, and a plurality of different kinases are immobilized on the bottom surface of the well. The substrate and the kinases are in proximity with each other sufficient for the occurrence of an enzymatic reaction. In more specific embodiments, each kinase of the plurality of kinases is immobilized at a different position of the bottom surface of the well in a positionally addressable fashion.

In certain embodiments, the kinases of the plurality of kinases are derived from a single species. In other embodiments, the kinases of the plurality of kinases are derived from different species. In more specific embodiments, the kinases of the plurality of kinases are derived from a prokaryotic organism. In other embodiments, the kinases of the plurality of kinases are derived from an organism such as, but not limited to, yeast, Caenorhabditis elegans, Drosophila melanogaster, mouse, rat, horse, chimpanzee, or human.

In certain embodiments, a plurality of immobilized kinases includes one or more kinase from each branch of a kinome. In certain, more specific embodiments, a plurality of immobilized kinases includes one or more kinases from each branch of a mammalian kinome, such as a human kinome. A kinome includes all of the kinases within a species of organism.

In a specific embodiment, the kinase assays of the invention can be used to analyze the activity of kinases in a particular biological sample. This method is useful for, e.g., defining a pathological state of a cell based on the level of kinase activity as opposed to abundance of mRNA or protein. In specific embodiments, kinases whose activity is upregulated or downregulated in a preneoplastic, a neoplastic or a cancerous cell can be identified. Kinases whose activity is modulated in a cell of a specific disease or disorder compared to a normal cell are candidates for drug targets to identify drugs for treating the disease or disorder.

In certain embodiments, a plurality of different substrates is immobilized on the surface of a solid support and the extract of a cell is also immobilized on the surface of the solid support such that at least one substrate of the plurality of different substrates is in proximity with the extract sufficient for the occurrence of a kinase reaction between the substrate and the extract. In a specific embodiment, at least one substrate of the plurality of different substrates is a known substrate of a kinase reaction. In certain embodiments, the different substrates are organized in a positionally addressable array. This embodiment is useful for assessing kinase activities in a particular type of cell, wherein type of cell can refer to developmental state of the cell, stage of the cell cycle in the cell, or whether the cell is derived from a pathological tissue, e.g., is neoplastic or cancerous. In this embodiment, kinase activity is defined by the substrate. In certain, more specific embodiments, the plurality of different substrates is immobilized several times at different positions of the surface of the solid support. In certain embodiments, extracts from different types of cells are immobilized at the different positions such that each plurality or at least some of the pluralities of different substrates are in contact with a different cellular extract. In certain embodiments, each plurality or at least some of the pluralities of different substrates are in proximity with cellular extract from the same type of cell sufficient for the occurrence of a kinase reaction between the substrates of the pluralities and the kinases of the cellular extract. In certain embodiments, different reaction mixtures, i.e., reaction mixtures providing different conditions and/or cofactors, are contacted with the different pluralities of different substrates.

The invention also relates to protein microarrays. In certain embodiments the invention provides a positionally addressable array comprising at least one known kinase and at least one candidate substrate of the kinase, wherein (i) the kinase and the substrate are immobilized on the surface of a solid support; (ii) the kinase and the substrate are in proximity sufficient for the occurrence of the enzymatic reaction catalyzed by the kinase between the kinase and the substrate; and (iii) the kinase and the substrate are not identical to each other. In other embodiments, the positionally addressable array of the invention comprises at least one known substrate of a kinase reaction and at least one candidate kinase for the catalysis of the kinase reaction, wherein (i) the kinase and the substrate are immobilized on the surface of a solid support; (ii) the kinase and the substrate are in proximity sufficient for the occurrence of the enzymatic reaction between the kinase and the substrate; and (iii) the kinase and the substrate are not identical to each other. In even other embodiments, a positionally addressable array comprises at least one known substrate of a kinase reaction and at least one kinase that is known to catalyze the enzymatic reaction, wherein (i) the kinase and the substrate are immobilized on the surface of a solid support; (ii) the kinase and the substrate are in proximity sufficient for the occurrence of the enzymatic reaction between the kinase and the substrate; and (iii) the enzyme and the substrate are not identical to each other.

In certain embodiments, a plurality of kinases and a substrate are immobilized on the microarrays of the invention. The plurality of kinases can be a selection of kinases, such as, but not limited to kinases derived from a single species, kinases of a particular enzymatic activity, and kinases derived from a specific cellular extract. The microarrays of the invention can be coated with a substrate, or the substrate can be deposited on different spots of the surface of the solid support and the kinases of the plurality of kinases are deposited on top of the substrate. In certain more specific embodiments, the substrate is a known substrate of the kinase reaction to be assayed. In certain, more specific embodiments, each kinase of the plurality of kinases is immobilized at a different position of the surface of the solid support. Alternatively, the plurality of kinases is deposited first and the substrate is deposited subsequently on top of the kinases. In certain embodiments, the plurality of kinases is organized in a positionally addressable array.

In other embodiments, a plurality of substrates and a kinase are immobilized on the microarrays of the invention. The plurality of substrates can be a selection of proteins, peptides, sugars, polysaccharides, small organic molecules, inorganic molecules, DNA or RNA. The microarrays of the invention can be coated with the kinase, or the kinase can be deposited on different spots of the surface of the solid support and the substrates of the plurality of substrates are deposited on top of the kinase. Alternatively, the plurality of substrates is deposited first and the kinase is deposited subsequently on top of the substrates.

In certain embodiments, the microarrays of the invention have wells. In certain embodiments, at least one well is pre-coated or pre-deposited with a substrate and a plurality of different kinases is deposited on the surface of the solid support in the well such that a substrate and a kinase are in proximity with each other sufficient for the occurrence of an enzymatic reaction between the kinase and the substrate. In certain embodiments, at least one well is pre-coated or pre-deposited with a kinase and a plurality of different substrates is deposited on the surface of the solid support in the well such that a substrate and a kinase are in proximity with each other sufficient for the occurrence of an enzymatic reaction between the kinase and the substrate. In certain, more specific embodiments, the substrates are potential substrates of the kinase. In other embodiments, the substrates are known substrates of the kinase.

In certain embodiments, each well of a microarray of the invention has the same combination of substrates and kinases immobilized to the surface of the solid support within the well. In this embodiment, each well of the microarray can be filled with a different reaction buffer such that the kinase reaction(s) can be monitored under a plurality of different reaction conditions; in the presence and absence, respectively, of a plurality of different test molecules; or in the presence and absence, respectively, of different cofactors.

The invention also provides kits for carrying out the assay regimens of the invention and for manufacturing the microarrays of the invention. In a specific embodiment, kits of the invention comprise one or more arrays of the invention. Such kits may further comprise, in one or more containers, reagents useful for assaying biological activity of a kinase, reagents useful for assaying interaction of a substrate and a kinase, reagents useful for assaying the biological activity of a kinase having a biological activity of interest. The reagents useful for assaying biological activity of a kinase, or assaying interactions between a probe and kinase, can be contained in each well or selected wells on the protein chip. Such reagents can be in solution or in solid form. The reagents may include either or both kinases and the substrates required to perform the assay of interest.

In one embodiment, a kit comprises one or more protein microarrays of the invention. In certain embodiments, the kinases and substrates are already immobilized onto the surface of the solid support. In another embodiment, reagents are provided in the kit that can be used for immobilizing substrate and kinase onto the surface of the solid support.

In certain embodiments, the substrate is different from the kinases of the plurality of kinases.

In certain embodiments, the invention provides a method for assaying an kinase reaction, the method comprising: (a) incubating at least one kinase, at least one first substrate, and at least one second substrate under conditions conducive to the occurrence of an enzymatic reaction between the kinase and the first or the second substrate, wherein (i) the kinase, the first substrate and the second substrate are immobilized on the surface of a solid support; (ii) the kinase, the first substrate and the second substrate are in proximity sufficient for the occurrence of said enzymatic reaction; (iii) the kinase and the first substrate are not identical and (iv) the kinase and the second substrate are not identical; and (b) determining whether said enzymatic reaction occurs.

### Solid Support and Immobilization of Substrate and Protein

In the methods and microarrays of the invention, at least one substrate and at least one kinase are immobilized on the surface of a solid support such that substrate and kinase are in proximity sufficient for the occurrence of an enzymatic reaction. The substrate is a candidate substrate or a known substrate of the enzymatic reaction. The kinase is a candidate enzyme or an enzyme known to catalyze the enzymatic reaction of interest.

The substrate and the kinase can be immobilized to the surface of the solid support by any method known to the skilled artisan. In certain embodiments, the substrate is immobilized before the kinase is immobilized. In other embodiments, the kinase is immobilized before the substrate is immobilized. The suitability of a specific method of immobilizing a kinase or a substrate may depend on the molecular nature of the kinase or substrate. If the substrate is a proteinaceous substrate, e.g., a protein or a peptide, any method known to the skilled artisan can be used to immobilize a protein to the surface of a solid support. If the substrate is not a proteinaceous substrate, any method known to the skilled artisan can be used to immobilize a molecule of that type of molecules to surface of a solid support.

In certain embodiments of the invention, the substrate and the kinase are immobilized on the surface of the solid support such that substrate and kinase are in proximity with each other sufficient for the occurrence of the enzymatic reaction to be assayed. Typically, when the substrate and the kinase are in sufficient proximity immobilized on the surface of the solid support, physical contact between the substrate and the kinase occurs during incubation under conditions conducive to the occurrence of an enzymatic reaction between the kinase and the substrate. In certain embodiments of the invention, the substrate and the kinase are immobilized on the surface of the solid support such that substrate and kinase are in physical contact with each other.

In certain embodiments, the substrate is purified. In certain embodiments, the kinase is purified. In certain embodiments, the substrate and the kinase are purified.

In certain embodiments, the surface of a solid support is coated or deposited with a mixture of at least 2, 3, 4, 5, 10, 15, 20, 25, 50 or 100 different substrates. In certain embodiments, the surface of a solid support is coated or deposited with a mixture of at most 2, 3, 4, 5, 10, 15, 20, 25, 50 or 100 different substrates. In certain embodiments, a plurality of different mixtures of substrates is immobilized on the surface of the solid support.

The solid support can be constructed from materials such as, but not limited to, silicon, glass, quartz, polyimide, acrylic, polymethylmethacrylate (by way of example only, LUCITE®), ceramic, gold, nitrocellulose, amorphous silicon carbide, polystyrene, and/or any other material suitable for microfabrication, microlithography, or casting. For example, the solid support can be a hydrophilic microtiter plate (by way of example only, MILLIPORE™) or a nitrocellulose-coated glass slide. In a specific embodiment, the solid support is a nitrocellulose-coated glass slide. Nitrocellulose-coated glass slides for making protein (and DNA) microarrays are commercially available (e.g., from Schleicher & Schuell (Keene, N.H.), which sells glass slides coated with a nitrocellulose based polymer (Cat. no. 10 484 182)). In a specific embodiment, each kinase is spotted onto the nitrocellulose-coated glass slide using an OMNIGRID™ (GeneMachines, San Carlos, Calif.). The present invention contemplates other solid supports useful for constructing a protein chip, some of which are disclosed, for example, in International Patent Application publication WO 01/83827 which is incorporated herein by reference in its entirety.

In one embodiment, the solid support is a flat surface such as, but not limited to, a glass slide. Dense protein arrays can be produced on, for example, glass slides, such that assays for the presence, amount, and/or functionality of kinases can be conducted in a high-throughput manner.

In certain, more specific embodiments, the solid support is a glass slide that has been pre-treated with an aldehyde, such as paraformaldehyde or formaldehyde. In certain embodiments, the solid support is an aldehyde treated slide is obtained from TeleChem International, Inc. In other embodiments, the solid support is a nitrocellulose coated slide (Schleicher & Schuell). In other embodiments, the solid support is coated with an amino-silane surface (GAPS slide obtained from Corning®).

In certain embodiments, after immobilizing the substrates and the proteins, the chip is blocked. Any blocking agent known to the skilled artisan can be used with the methods of the invention. In a specific embodiment, Bovine Serum Albumin, glycine or a detergent (e.g., Tween20) can be used as a blocking agent. In certain other embodiments the chips are not blocked.

In a particular embodiment, the solid support comprises a silicone elastomeric material such as, but not limited to, polydimethylsiloxane (PDMS). An advantage of silicone elastomeric materials is their flexible nature.

In another particular embodiment, the solid support is a silicon wafer. The silicon wafer can be patterned and etched (see, e.g., G. Kovacs, 1998, Micromachined Transducers Sourcebook, Academic Press; M. Madou, 1997, Fundamentals of Microfabrication, CRC Press). The etched wafer can also be used to cast the microarrays to be used with the invention.

Accordingly, in certain embodiments, the plurality of kinases is applied to the surface of a solid support, wherein the density of the sites at which the kinases are applied is at least 1 site/cm², 2 sites/cm², 5 sites/cm², 10 sites/cm², 25 sites/cm², 50 sites/cm², 100 sites/cm², 1000 sites/cm², 10,000 sites/cm², 100,000 sites/cm², 1,000,000 sites/cm², 10,000,000 sites/cm², 25,000,000 sites/cm², 10,000,000,000 sites/cm², or 10,000,000,000,000 sites/cm². Each individual kinase is preferably applied to a separate site on the chip. In certain specific embodiments, the identities of the kinase(s) at each site on the chip is/are known. In certain other embodiments, a plurality of substrates is applied to the surface of a solid support, wherein the density of the sites at which substrates are applied is at least 1 site/cm², 2 sites/cm², 5 sites/cm², 10 sites/cm², 25 sites/cm², 50 sites/cm², 100 sites/cm², 1000 sites/cm², 10,000 sites/cm², 100,000 sites/cm², 1,000,000 sites/cm², 10,000,000 sites/cm², 25,000,000 sites/cm², 10,000,000,000 sites/cm², or 10,000,000,000,000 sites/cm². Each individual substrate sample is preferably applied to a separate site on the chip. In certain specific embodiments, the identities of the substrates at each site on the chip are known, i.e., the chip is a positionally addressable array.

In certain aspects of the invention, a population of identical kinases is immobilized on a specific region on the surface of the solid support. Different populations of identical kinases can be immobilized on different specific regions of the surface of the solid support. The regions can be separated for example, by less than 10 millimeters, less than 1 millimeter, less than 500 microns, or less than 100 microns. In certain embodiments, the different regions containing populations of identical kinases can be formed by printing the kinases to the surface of the solid support using a microarray printer.

In certain embodiments, a plurality of different kinases is applied to the surface, wherein the surface is either pre-coated with a substrate or pre-deposited with substrate. If the surface is pre-deposited with a substrate, care should be taken that each of the different kinases is deposited on top of the sites where a substrate is present. In certain other embodiments, a plurality of different substrates is applied to the surface, wherein the surface is either pre-coated with a kinase or pre-deposited with a kinase. If the surface is pre-deposited with a kinase, care should be taken that each of the different substrates is deposited on top of the sites where the kinase is present. The substrate can be a candidate substrate for the kinase reaction to be assayed.

In certain embodiments, a substrate and a kinase are immobilized on the surface of a solid support, wherein the solid support has wells. In certain embodiments, a plurality of different kinases or different substrates is deposited on the surface of the solid support within each well, thereby creating an array within each well such that each feature of the microarray is in a different well. In other embodiments, a plurality of different kinases or different substrates is deposited onto the surface of the solid support such that each well harbors a plurality of different kinases or substrates. The performance of the enzymatic reaction on a solid support with wells has the advantage that different reaction solutions can be added at the same time onto one solid support (e.g., on one slide). Another advantage of wells over flat surfaces is an increased signal-to-noise ratio. Wells allow the use of larger volumes of reaction solution in a denser configuration, and therefore greater signal is possible. Furthermore, wells decrease the rate of evaporation of the reaction solution from the chip as compared to flat surface arrays, thus allowing longer reaction times. Another advantage of wells over flat surfaces is that the use of wells permit association studies using a specific volume of reaction volume for each well on the chip, whereas the use of flat surfaces usually involves indiscriminate probe application across the whole surface. The application of a defined volume of reaction buffer can be important if a reactant that is supplied in the reaction buffer is being depleted during the course of the reaction. In such a scenario, the application of a defined volume allows for more reproducible results. The use of microlithographic and micromachining fabrication techniques (see, e.g., International Patent Application publication WO 01/83827, which is incorporated herein by reference in its entirety) can be used to create well arrays with a wide variety of dimensions ranging from hundreds of microns down to 100 nm or even smaller, with well depths of similar dimensions. In addition, the solid supports with wells created by microlithographic and micromachining fabrication techniques can be used as master molds to cast solid supports with wells out of polymeric material. In one embodiment, a silicon wafer is micromachined and acts as a master mold to cast a support with wells of 400 μm diameter that are spaced 200 μm apart, for a well density of about 277 wells per cm², with individual well volumes of about 30 nl for 100 μm deep wells (see, e.g., International Patent Application publication WO 01/83827, which is incorporated herein by reference in its entirety).

In certain embodiments, the wells of a microarray of the invention have depth. In other embodiments, the wells of a microarray of the invention do not have depth. In a nonlimiting example, the different wells are separated by barriers wherein the barrier comprises a different surface material than the surface material of the well. By way of example only, the wells are constituted by an area on the solid support that is a glass surface and the barriers are constituted by a surface material which is hydrophobic including, but not limited to, teflon. Such slides can be obtained, e.g., from Erie Scientific Company, NH. Without being bound by theory, the difference in surface tension provided by the different surface materials ensures that a liquid from one well will not leak into a neighboring well.

In one embodiment, the solid support comprises gold. In a preferred embodiment, the solid support comprises a gold-coated slide. In another embodiment, the solid support comprises nickel. In another preferred embodiment, the solid support comprises a nickel-coated slide. Solid supports comprising nickel are advantageous for purifying and attaching fusion proteins having a poly-histidine tag (“His tag”). In another embodiment, the solid support comprises nitrocellulose. In another preferred embodiment, the solid support comprises a nitrocellulose-coated slide.

The kinases and substrates can be bound directly to the solid support, or can be attached to the solid support through a linker molecule or compound. The linker can be any molecule or compound that derivatizes the surface of the solid support to facilitate the attachment of proteins and/or substrates to the surface of the solid support. The linker may covalently or non-covalently bind the kinases or substrates to the surface of the solid support. In addition, the linker can be an inorganic or organic molecule. In certain embodiments, the linker may be a silane, e.g., sianosilane, thiosilane, aminosilane, etc. Compounds useful for derivatization of a protein chip are also described in International Patent Application publication WO 01/83827, which is incorporated herein by reference in its entirety.

Accordingly, in one embodiment, the kinases and/or substrates are bound non-covalently to the solid support (e.g., by adsorption). Kinases and/or substrates that are non-covalently bound to the solid support can be attached to the surface of the solid support by a variety of molecular interactions such as, for example, hydrogen bonding, van der Waals bonding, electrostatic, or metal-chelate coordinate bonding. In a particular embodiment, kinases and/or substrates are bound to a poly-lysine coated surface of the solid support. In addition, as described above, in certain embodiments, the kinases and/or substrates are bound to a silane (e.g., sianosilane, thiosilane, aminosilane, etc.) coated surface of the solid support.

In addition, crosslinking compounds commonly known in the art, such as homo- or heterofunctional crosslinking compounds may be used to attach proteins and/or substrates to the solid support via covalent or non-covalent interactions. Such crosslinking agents include, but are not limited to, bis[sulfosuccinimidyl]suberate, N-[gamma-maleimidobutyryloxy]succinimide ester, and 1-ethyl-3-[3-dimethylaminopropyl]carbodiimide).

In another embodiment, kinases and/or substrates of the protein chip are bound covalently to the solid support. In other embodiments, kinases and/or substrates can be bound to the solid support by receptor-ligand interactions, which include interactions between antibodies and antigens, DNA-binding proteins and DNA, enzyme and substrate, avidin (or streptavidin) and biotin (or biotinylated molecules), and interactions between lipid-binding proteins and phospholipids (or membranes, vesicles, or liposomes comprising phospholipids).

Purified kinases and/or substrates can be placed on an array using a variety of methods known in the art. In one embodiment, the kinases and/or substrates are deposited onto the surface of a solid support. In a further embodiment, the kinases and/or substrates are attached to the solid support using an affinity tag. In a specific embodiment, an affinity tag different from that used to purification of the kinase or substrate is used for immobilizing the kinase or substrate. If two different tags are used further purification is achieved when building the protein array.

In a specific embodiment, kinases and/or substrates have an affinity for a compound that is attached to the surface of the solid support. Suitable compounds include, but are not limited to, trypsin/anhydrotrypsin, glutathione, immunoglobulin domains, maltose, nickel, or biotin and its derivatives, which bind to bovine pancreatic trypsin inhibitor, glutathione-S-transferase, Protein A or antigen, maltose binding protein, poly-histidine (e.g., HisX6 tag), and avidin/streptavidin, respectively. For example, Protein A, Protein G and Protein A/G are proteins capable of binding to the Fc portion of mammalian immunoglobulin molecules, especially IgG. These proteins can be covalently coupled to, for example, a Sepharose® support. In a specific embodiment, the kinases are bound to the solid support via His tags, wherein the solid support comprises a flat surface. In a preferred embodiment, the kinases are bound to the solid support via His tags, wherein the solid support comprises a nickel-coated glass slide.

In certain embodiments, proteins and/or substrates are expressed as fusion proteins, wherein the protein and/or substrate is fused to a bifunctional tag. In an example of such an embodiment, the protein and/or substrate is fused to an intein and a chitin binding domain. In a more specific embodiment, the proteins and/or substrates are expressed using the IMPACT™-CN system from New England Biolabs Inc. In the presence of thiols such as DTT, b-mercaptoethanol or cysteine, the intein undergoes specific self-cleavage which releases the target protein from the chitin-bound intein tag.

The protein chips to be used with the present invention are not limited in their physical dimensions and can have any dimensions that are useful. Preferably, the protein chip has an array format compatible with automation technologies, thereby allowing for rapid data analysis. Thus, in one embodiment, the protein microarray format is compatible with laboratory equipment and/or analytical software. In a preferred embodiment, the protein chip is the size of a standard microscope slide. In another preferred embodiment, the protein chip is designed to fit into a sample chamber of a mass spectrometer.

In specific embodiments, kinases and/or substrates are applied to a flat surface, such as, but not limited to, glass slides. Kinases and/or substrate are bound covalently or non-covalently to the flat surface of the solid support. The kinases and/or substrate can be bound directly to the flat surface of the solid support, or can be attached to the solid support through a linker molecule or compound. The linker can be any molecule or compound that derivatizes the surface of the solid support to facilitate the attachment of proteins and/or substrate to the surface of the solid support. The linker may covalently or non-covalently bind the kinases and/or substrate to the surface of the solid support. In addition, the linker can be an inorganic or organic molecule. By way of example only, specific linkers are compounds with free amines. Preferred among linkers is 3-glycidooxypropyltrimethoxysilane (GPTS).

In a non-limiting embodiment, by way of example only, kinases are immobilized on the solid support using the following procedure: briefly, after washing with 100% ethanol (EtOH) three times at room temperature, the chips (e.g., chips made of polydimethylsiloxane or glass slides) are immersed in 1% GPTS solution (95% ethanol (EtOH), 16 mM acetic acid (HOAc)) with shaking for 1 hr at room temperature. After three washes with 95% EtOH, the chips are cured at 135° C. for 2 hrs under vacuum. Cured chips can be stored in dry Argon for months 12. To attach kinases and substrates to the chips, kinase solutions are added to the wells and incubated on ice for 1 to 2 hours. After rinsing with cold HEPES buffer (10 mM HEPES, 100 mM NaCl, pH 7.0) three times, the wells are blocked with 1% BSA in PBS (Sigma, USA) on ice for >1 hr. Because of the use of GPTS, any reagent containing primary amine groups is avoided.

Printing of one or more kinases or one or more substrates can be accomplished, for example, by microspotting, which encompasses deposition technologies that enable automated microarray production by printing small quantities of pre-made biochemical substrates onto solid surfaces. Printing is accomplished by direct surface contact between the printing substrate and a delivery mechanism, such as a pin or a capillary. Robotic control systems and multiplexed printheads allow automated microarray fabrication.

Ink jet technologies utilize piezoelectric and other forms of propulsion to transfer biochemical substrates from miniature nozzles to solid surfaces. Using piezoelectricity, the sample is expelled by passing an electric current through a piezoelectric crystal that expands to expel the sample. Piezoelectric propulsion technologies include continuous and drop-on-demand devices. Examples of the use of ink jet technology include U.S. Pat. No. 5,658,802 (issued Aug. 19, 1997).

In another embodiment, protein-containing cellular material, such as but not limited to vesicles, endosomes, subcellular organelles, and membrane fragments, can be placed on the protein chip. In another embodiment, a whole cell is placed on the protein chip. In a further embodiment, the protein, protein-containing cellular material, or whole cell is attached to the solid support of the protein chip. In a specific embodiments, the protein, protein-containing cellular material, or whole cell is attached to the surface of the solid support that is coated or predeposited with substrate.

Furthermore, proteins, substrate, protein- or substrate-containing cellular material, or cells can be embedded in artificial or natural membranes prior to or at the time of placement on the protein chip. Embedding kinases in membranes is the preferred embodiment, if the kinase assumes its enzymatically active conformation preferentially in a membrane. In another embodiment, proteins, protein-containing cellular material, or cells can be embedded in extracellular matrix component(s) (e.g., collagen or basal lamina) prior to or at the time of placement on the protein chip.

The kinases and/or substrates are bound covalently or non-covalently to the surface of wells on the solid support. In more specific embodiments, the kinase is bound covalently to the surface and the substrate is bound non-covalently to the surface. In other embodiments, the kinase is bound non-covalently to the surface and the substrate is bound covalently to the surface. In other embodiments, both substrate and kinase are bound covalently to the surface. In other embodiments, both substrate and kinase are bound non-covalently to the surface. The kinases and/or substrates can be bound directly to the surface of the solid support, or can be attached to the solid support through a linker molecule or compound. The linker can be any molecule or compound that derivatizes the surface of the solid support to facilitate the attachment of proteins or substrates to the surface of the solid support. The linker may covalently bind the kinases and/or substrates to the surface of the solid support or the linker may bind via non-covalent interactions. In addition, the linker can be an inorganic or organic molecule. By way of example only, linkers are compounds with free amines, with a preferred linkers being 3-glycidooxypropyltrimethoxysilane (GPTS).

Kinases and/or substrates which are non-covalently bound to the surface of the solid support may utilize a variety of molecular interactions to accomplish attachment to surface of the solid support such as, for example, hydrogen bonding, van der Waals bonding, electrostatic, or metal-chelate coordinate bonding. Further, DNA-DNA, DNA-RNA and receptor-ligand interactions are types of interactions that utilize non-covalent binding. Examples of receptor-ligand interactions include interactions between antibodies and antigens, DNA-binding proteins and DNA, enzyme and substrate, avidin (or streptavidin) and biotin (or biotinylated molecules), and interactions between lipid-binding proteins and phospholipid membranes or vesicles. For example, proteins and/or substrates can be expressed with fusion protein domains that have affinities for a binding partner that is attached to the surface of the solid support. Suitable binding partners for fusion protein binding include trypsin/anhydrotrypsin, glutathione, immunoglobulin domains, maltose, nickel, or biotin and its derivatives, which bind to bovine pancreatic trypsin inhibitor, glutathione-S-transferase, antigen, maltose binding protein, poly-histidine (e.g., HisX6 tag), and avidin/streptavidin, respectively.

In certain embodiments, the proteins and/or the substrate is immobilized to the solid support via a peptide tag, wherein the affinity binding partner for the tag is attached (covalently or non-covalently) to the solid support. For a more detailed description of peptide tags see section 5.5.1.

In certain embodiments, a kinase is immobilized directly on the surface of the solid support. In other embodiments, a kinase is immobilized via a linker molecule to the solid support. In certain, more specific embodiments, the distance between a kinase and the surface of a solid support is at most 0.1 nm, 1 nm, 5 nm, 10 nm, 15 nm, 25 nm, 50 nm, 100 nm, 1 μm or at most 5 μm. In certain embodiments, the distance between the kinase and the surface of the solid support is at least 0.1 nm, 1 nm, 5 nm, 10 nm, 15 nm, 25 nm, 50 nm, 100 nm, 1 μm or at least 5 μm. In certain embodiments, a kinase is immobilized to the underivatized surface of a solid support. In a more specific embodiment, a kinase is immobilized to the underivitized glass surface of a solid support.

In certain embodiments, the substrate is immobilized directly on a surface of a solid support. In other embodiments, a substrate is immobilized via a linker molecule to a solid support. In certain, more specific embodiments, the distance between a substrate and the surface of a solid support is at most 0.1 nm, 1 nm, 5 nm, 10 nm, 15 nm, 25 nm, 50 nm, 100 nm, 1 μm or at most 5 μm. In certain embodiments, the distance between a substrate and the surface of a solid support is at least 0.1 nm, 1 nm, 5 nm, 10 nm, 15 nm, 25 nm, 50 nm, 100 nm, 1 μm or at least 5 μm. In certain embodiments, a substrate is immobilized to the underivatized surface of a solid support. In a more specific embodiment, the substrate is immobilized to the underivitized glass surface of a solid support.

In certain embodiments, a substrate and a kinase are immobilized directly on the surface of the solid support. In other embodiments, a substrate and a kinaseare immobilized via a linker molecule to the solid support. In certain, more specific embodiments, the distance between a substrate and the surface of the solid support and the distance between a kinase and the surface of the solid support (i.e., the length of the linker molecule, or the distance by which the linker distances the substrate or the kinase from the solid support) is at most 0.1 nm, 1 nm, 5 nm, 10 nm, 15 nm, 25 nm, 50 nm, 100 nm, 1 μm or at most 5 μm. In certain embodiments, the distance between a substrate and the surface of the solid support and the distance between a kinase and the surface of the solid support is at least 0.1 nm, 1 nm, 5 nm, 10 nm, 15 nm, 25 nm, 50 nm, 100 nm, 1 μm or at least 5 μm. In certain embodiments, a substrate and a kinase are immobilized to the underivatized surface of the solid support. In a more specific embodiment, a substrate and a kinase are immobilized to the underivitized glass surface of a solid support.

The solid support can have a porous or a non-porous surface.

An aspect to be considered when choosing the surface chemistry for immobilizing substrate and a protein are background signals created by the surface.

Kinases can be immobilized in many ways on a surface. In certain embodiments, a substrate or a kinase can be immobilized reversibly. In other embodiments, a substrate or a kinase can be immobilized irreversibly. The goal of immobilizing a substrate and a kinase is to retain the kinase and the substrate in a defined region on the microarray. The kinase and/or the substrate can be encapsulated or entrapped in a porous surface or a vesicle. The kinase and/or the substrate can be kinetically trapped but has free molecules in equilibrium with surface-bound ones.

In certain embodiments, the different kinases and/or the different substrates on the surface of a solid support are present in approximately equimolar amounts. Without being bound by theory, using approximately equimolar amounts facilitates the quantification of the results obtained.

In certain embodiments of the invention, the amount of a kinase or a substrate is present on the surface of a solid support is at least 10⁻¹² mol, 10⁻¹¹ mol, 10⁻¹⁰ mol, 10⁻⁹ mol, 10⁻⁸ mol, 10⁻⁷ mol, 10⁻⁶ mol, 10⁻⁵ mol, 10⁻⁴ mol, 10⁻³ mol, 10⁻² mol, or at least 10⁻¹ mol. In certain embodiments of the invention, the amount of a protein or a substrate is present on the surface of a solid support is at most 10⁻¹² mol, 10⁻¹¹ mol, 10⁻¹⁰ mol, 10⁻⁹ mol, 10⁻⁸ mol, 10⁻⁷ mol, 10⁻⁶ mol, 10⁻⁵ mol, 10⁻⁴ mol, 10⁻³ mol, 10⁻² mol, or at least 10⁻¹ mol.

Illustrative examples of immobilizing a kinase and a substrate include, but are not limited to,

1. Immobilization by specific covalent bonds, such as disulfide with a cysteine, or non-specific covalent bonds, such as a Schiff base, formed between a protein or a substrate and the surface of the solid support (e.g., a slide).

2. Immobilization by adsorption of a kinase or a substrate directly onto the surface of the solid support.

3. Immobilization by specific non-covalent interactions between a substrate or a protein and the surface, such as His-tagged proteins or substrates and Nickel surfaces.

4. Immobilization indirectly by interactions of a kinase or a substrate with immobilized molecules, including proteins, lipids, nucleic acids and carbohydrates.

5. The interactions of a kinase or a substrate with immobilized molecules can be specific, such as antibody/antigen or streptavidin/biotin.

6. The interactions of a kinase or a substrate with immobilized molecules can be non-specific.

7. Immobilization by cross linking to a matrix on the slide.

8. Immobilization by entrapment in a matrix on the slide.

9. The matrix can be made of polymers. The polymerization and/or the cross linking can occur before, during and after the printing of proteins.

10. The matrix can be made of interactions of non-covalent natures, such as hydrogen bonds and van der Waals interactions, between the same or different types of molecules.

11. A kinase or a substrate to be immobilized can be part of the matrix formation.

12. Immobilization by encapsulation of a kinase or a substrate in molecular-scale compartments, such as liposomes, vesicles or micelles, which are covalently or non-covalently attached to a surface.

13. Immobilization by protein aggregation, cross-linking, precipitation or denaturation on the surface of a solid support.

14. Immobilization by coating a kinase or substrate on a support surface and allowing the kinase or substrate to non-covalently bind to the surface.

In certain embodiments, substrate and kinase are immobilized by different procedures. In certain other embodiments, substrate and protein are immobilized by the same procedure.

Covalent bonding or other strong interactions between a kinase and the surface of a solid support may modify the structure and thus function of a kinase. Thus, the skilled artisan can, e.g., by means of structural prediction programs, available structures of kinases or experimental determination of a structure determine which region of a kinase is best suited to be in contact with the surface or the linker. In an illustrative embodiment, a kinase is known to have two structural domains, a first domain with catalytic activity and a second domain. In a specific embodiment, the second domain is linked to the surface of the solid support. In another embodiment, the first domain is linked to the surface of the solid support. Without being bound by theory, immobilization directly through the domain with the catalytic activity may inhibit activity. Immobilization of catalytic domains may not be desirable. Instead, immobilization through a fused domain or protein may offer better activity.

Other factors to be considered in generating the microarrays to be used with the methods of the invention are: Enzymatic activities increase with the amounts of kinases and substrates. Higher activities will also result if the effective concentrations of enzyme and substrate are higher. Proteins may denature at liquid/solid or air/liquid interface, resulting in less activity. Restricting enzyme or substrate conformations on a surface may reduce productive interactions between the molecules. The diffusion rate of large molecules is low, and the rate of reaction can be diffusion-limited.

In certain embodiments, slides with high protein binding capacities are used to increase local kinase and/or substrate concentrations. Without being limited by theory, bringing kinases and substrates into closer proximity may increase the effective concentrations. Immobilization of a kinase or a substrate by non-specific adsorption may denature a kinase. Interactions between slide surface and a kinase or a substrate may reduce their diffusion rates. The interactions increase with larger surface areas as on surfaces made of porous materials or matrices. Further, entrapment or immobilization using indirect methods may be less disruptive to the enzymes.

For the microarray assay to work effectively, the background signals from labeled molecules need to be minimized. In certain embodiments, the interactions between the surface and a labeled molecule that is used in the kinase reaction can be blocked with a non-labeled molecule before or during the kinase reaction to minimize background. The binding kinetics of molecules often depend on the concentrations of the probe, available slide surface areas for binding, temperature as well as the specific chemistry. Slides made of matrices or porous materials have much higher surface areas and thus potentially more interactions with the labeled molecules.

In certain embodiments, surfaces having slower binding kinetics compared to the assay time may offer better signal to background.

In certain embodiments, surfaces with lower protein binding capacities may reduce background. However, the binding capacity must be weighed with the sensitivity of the enzymatic assay as a reduction in kinase will also reduce signal intensity.

Other considerations include that surface chemistry also affects the making of protein microarrays. The surface properties, such as hydrophobicity, flatness, and homogeneity, influence the amount of proteins delivered to the slide and the size and morphology of the spots. These factors will ultimately affect the assay sensitivity and reproducibility.

Typically, in the methods of the present invention, a substrate (e.g., a substrate of a kinase reaction) and a kinase (e.g., an enzyme) are immobilized on the surface of a solid support before the kinase and the substrate are incubated under conditions conducive to the occurrence of an enzymatic reaction between the kinase and the substrate. Furthermore, the kinase and the substrate remain immobilized during at least a portion of the incubation step on the surface of the solid support at the location at which they were immobilized before the incubation step, for at least a time sufficient for the enzymatic reaction between the substrate and the kinase to take place. In certain embodiments of the methods of the present invention, a substrate (e.g., a substrate of an kinase reaction) and a kinase (e.g., an enzyme) are immobilized on the surface of a solid support in a manner such that they remain immobilized throughout the incubation step, at the same location at which they were immobilized on the solid support before the incubation step, and optionally can remain immobilized during the determining step as well at the location. The immobilization of the substrate and the kinase before the incubation step provides a difference between the present invention and traditional solution based assays, in which both kinase and substrate are not immobilized before the incubation step.

Accordingly, an incubating step of a method of the invention can be performed with one aliquot of incubation buffer covering the entire surface of a solid support containing multiple different immobilized kinases and/or multiple different immobilized substrates. Alternatively, an incubation step (a) of a method of the invention can be performed with one aliquot of incubation buffer covering the entire surface of a region of a solid support containing, wherein the region includes multiple different immobilized kinases and/or multiple different immobilized substrates.

In an illustrative example, a mixture of five different substrates is immobilized on the surface of a solid support such that the surface of the solid support is coated with the mixture of the five different substrates. In addition, for example five hundred different kinases are immobilized on the surface of the solid support in a positionally addressable fashion, for example by printing the kinases on the solid support that has been coated with the mixture of substrates. Thus, 2500 different kinase-substrate combinations are generated on the surface of the solid support, wherein the kinase at any position on the surface can be identified because it was immobilized in a positionally addressable fashion. For the incubating step in this illustrative example, all 2500 different kinase-substrate combinations are covered with one continuous aliquot of reaction buffer without any separation of reaction buffer over the surface of the solid support. The 2500 different substrate-kinase combinations remain immobilized before and throughout at least a portion of the incubation step. Without being bound by theory, because the kinases and the substrates are immobilized on the surface of the solid support, neither kinase nor substrate diffuses away from its original position on the surface of the solid support during at least a portion of the incubation step sufficient for an enzymatic reaction between the kinase and the substrate to occur. In certain aspects, repeating regions of the 2500 different immobilized kinase-substrate combinations are included on the surface of the same solid support. In these aspects, each different region containing the 2500 different substrate-kinase combinations can be covered with a different reaction buffer, for example where each different reaction buffer is identical except that it contains a different test molecule.

In another illustrative example, five different substrates are immobilized on the surface of a solid support by coating the substrates on the solid support, each different substrate is immobilized in a different region of the surface of the solid support. Thus, the surface of the solid support is coated with the different substrates. In addition, a plurality of five hundred different kinases is immobilized on the surface of the solid support in a positionally addressable fashion, such as by being deposited onto the surface of the solid support. Thus, 2500 different kinase-substrate combinations are generated on the surface of the solid support, wherein the kinase at any position on the surface can be identified because it was immobilized in a positionally addressable fashion. For the incubating step in this illustrative example, all 2500 different kinase-substrate combinations are covered with one continuous aliquot of reaction buffer without any separation of reaction buffer over the surface of the solid support.

The substrates and the kinases are immobilized before they are incubated under conditions conducive to the occurrence of an enzymatic reaction between a kinase and a substrate that are in proximity sufficient for the occurrence of the enzymatic reaction. Furthermore, the substrates and the kinases remain immobilized for at least a portion of the incubation step such that the enzymatic reaction occurs. Furthermore, in certain embodiments, depending for example on the specific method used to immobilize the kinases and the substrates, the kinases and the substrate can remain immobilized throughout the incubation step. However, for the present invention it is not necessary that the kinase remains immobilized throughout the incubating and determining steps, since a determination of whether the reaction occurs is typically made by detecting a reaction product, which typically remains immobilized throughout the incubation step.

In even another illustrative example, five different substrates are immobilized on the surface of the solid support, each different substrate forming a patch at a defined position of the surface of the solid support. In addition, five different kinases are immobilized on the surface of the solid support within each patch, also in a positionally addressable fashion. Thus, 25 different positionally addressable substrate-kinase combinations are generated on the surface of the solid support. For the incubating step, all 25 different combinations can be covered with one continuous aliquot of reaction buffer without any separation of reaction buffer over the areas of the different combinations.

In certain embodiments, the kinase (e.g., an enzyme) and the substrate (e.g., a substrate of the kinase) are immobilized on the surface of a solid support such that the kinase and the substrate remain continuously immobilized on the surface of the solid support after one or more washing steps. In certain, more specific embodiments, the kinase and the substrate remain immobilized on the surface of the solid support after at least one, at least two, at least three, at least four, at least five, at least six, at least seven, at least eight, at least nine, or at least ten washing steps. The washing steps are carried out under conditions that do not break covalent bonds. In other embodiments, the kinase is immobilized before an incubation step and remains immobilized on the surface of the solid support only for a period of time sufficient for the enzymatic reaction between the kinase and the substrate. In these embodiments, occurrence of the enzymatic reaction can be determined by detecting a product that is immobilized on the surface of the substrate at the location of the substrate.

In certain embodiments, the kinase (e.g., an enzyme) is immobilized on the surface of a solid support with a dissociation constant (i.e., dissociation from immobilized state into a liquid phase that covers the surface of the solid support) of less than 1000 μM, less than 100 μM, less than 10 μM, less than 1 μM, less than 0.1 μM, less than 0.01 μM, less than 0.001 μM, or less than 0.0001 μM, and the substrate (e.g., the substrate of the kinase) is immobilized on the surface of a solid support with a dissociation constant of less than 1000 μM, less than 100 μM, less than 10 μM, less than 1 μM, less than 0.1 μM, less than 0.01 μM, less than 0.001 μM, or less than 0.0001 μM. In certain embodiments, Phosphate Buffered Saline (PBS) is added to the surface of a solid support and the ratio between immobilized kinase and kinase that is dissolved in PBS can be determined. In certain embodiments, the ratio between immobilized kinase and kinase that is dissolved in PBS is at least 1:1; 10:1; 100:1; 10³:1; 10⁴:1; 10⁵:1; 10⁶:1; 10⁷:1; 10⁸:1; 10⁹:1; or at least 10¹⁰:1. In certain, more specific, embodiments, at least 1%, at least 5%, at least 10%, at least 25%, at least 50%, at least 75%, at least 90%, at least 95%, or at least 98% of the kinase that was immobilized before the enzymatic reaction and the substrate that was immobilized before the enzymatic reaction, respectively, remains immobilized after the enzymatic reaction.

In methods provided herein, the kinase (e.g., a candidate enzyme) and the substrate (e.g., a candidate substrate of the kinase) are typically immobilized on the surface of a solid support before an enzymatic reaction occurs between the kinase and the substrate. Occurrence of the enzymatic reaction can be determined by detecting an immobilized product at the same location on the surface of the solid support as was initially occupied by the substrate.

In certain embodiments, the kinase and the substrate are immobilized on the surface of a solid support before the incubation step and remain associated to the solid support for a storage period of at least one day, two days, three days, four days, five days, six days, one week, one month, two months, three months, four months, six months, or one year. In certain embodiments, an interaction between the kinase (e.g., a candidate enzyme) and the substrate (e.g., a candidate substrate) is not required for immobilization of the kinase and the substrate. In certain embodiments, immobilization of the kinase is independent of immobilization of the substrate, and, conversely, immobilization of the substrate is independent of immobilization of the kinase.

In certain aspects of the methods provided herein, after substrate(s) and/or kinase(s) are immobilized on a solid support, but before incubating the kinase(s) and the substrate(s) under conditions conducive to the occurrence of an enzymatic reaction between the kinase(s) and the substrate(s), the solid support is transported from a first location to a second location and/or between a first organization and a second organization. For example, the solid support with the immobilized kinase(s) and the immobilized substrate(s) can be shipped from a supplier to an end user. In certain aspects, methods provided herein include a purchase of the solid support containing the immobilized kinase(s) and/or the immobilized substrate(s) by a customer from a supplier and the transport of the solid support from the supplier to the customer. This purchase can be performed, for example, using an automated process, such as an internet-based process. The solid support with the immobilized kinase(s) and/or the immobilized substrate(s) can be transported in a storage buffer, for example a storage buffer that includes glycerol.

Kinase Reactions and their Quantification

In illustrative aspects of the invention that include a substrate that is MBP or a fragment or derivative thereof, the kinase included in a method, composition or kit herein is a tyrosine kinase. The tyrosine kinase, for example, can include a tyrosine kinase of Table 6. In certain illustrative aspects the tyrosine kinase is CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and/or YES1, which are identified as phosphorylating MBP in Table 6. In further illustrative embodiments, the tyrosine kinase is CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and/or SRC, identified as providing a strong phosphorylation signal in Table 6.

In certain embodiments, an enzymatic reaction of interest is performed wherein a substrate and a kinase are immobilized on the surface of a solid support such that the substrate and the kinase are in proximity sufficient for the occurrence of the enzymatic reaction. The reaction is performed by incubating the substrate and the kinase in a reaction mixture or reaction buffer that provides conditions conducive to the occurrence of the enzymatic reaction. The reaction conditions provided by the reaction buffer or mixture depend on the type of enzymatic reaction being performed and include, but are not limited to, salt concentration, detergent concentration, cofactors and pH. Other reaction conditions, such as temperature, also depend on the type of enzymatic reaction being performed.

Any enzymatic kinase reaction known to the skilled artisan can be performed with the methods of the invention. If the reaction involves more than one substrate, at least one substrate is immobilized, the other substrates can also be immobilized or can be in solution. In certain embodiments, if the enzymatic reaction involves one or more co-factors, such as, but not limited to, NAD, NADH or ATP, such a co-factor can be in solution or can also be immobilized on the surface of the solid support. Any method known to the skilled artisan can be used to visualize and quantitate the activity of the enzyme.

In certain embodiments, the enzymatic kinase reaction is performed such that the generation of the product of the reaction results in the emergence of a detectable signal. In certain embodiments, the enzymatic kinase reaction is performed such that an increase in concentration of the product of the reaction results in an increase of a detectable signal. In other embodiments, the enzymatic kinase reaction is performed such that an increase in concentration of the product of the reaction results in a decrease of a detectable signal. In certain embodiments, the enzymatic kinase reaction is performed such that an decrease of substrate concentration results in the increase or decrease of a detectable signal.

In certain embodiments, standard enzymatic assays that produce chemiluminescence or fluorescence are performed using a microarray, wherein kinase and substrate are immobilized on the surface of a solid support. Detection and quantification of an enzymatic reaction can be accomplished using, for example, photoluminescence, radioactivity, fluorescence using non-protein substrates, enzymatic color development, mass spectroscopic signature markers, and amplification (e.g., by PCR) of oligonucleotide tags. In a specific embodiment, peptides or other compounds released into solution by the enzymatic reaction of the array elements can be identified by mass spectrometry.

The types of assays to detect and quantify the products (or the decrease of substrate) of an enzymatic reaction fall into several general categories. Such categories of assays include, but not limited to: 1) using radioactively labeled reactants followed by autoradiography and/or phosphoimager analysis; 2) binding of hapten, which is then detected by a fluorescently labeled or enzymatically labeled antibody or high affinity hapten ligand such as biotin or streptavidin; 3) mass spectrometry; 4) atomic force microscopy; 5) fluorescent polarization methods; 6) rolling circle amplification-detection methods (Schweitzer et al., 2000, “Immunoassays With Rolling Circle DNA Amplification: A Versatile Platform For Ultrasensitive Antigen Detection”, Proc. Natl. Acad. Sci. USA 97:10113-10119); 7) competitive PCR (Fini et al., 1999, “Development of a chemiluminescence competitive PCR for the detection and quantification of parvovirus B19 DNA using a microplate luminometer”, Clin Chem. 45(9):1391-6; Kruse et al., 1999, “Detection and quantitative measurement of transforming growth factor-beta1 (TGF-beta1) gene expression using a semi-nested competitive PCR assay”, Cytokine 11(2):179-85; Guenthner and Hart, 1998, “Quantitative, competitive PCR assay for HIV-1 using a microplate-based detection system”, Biotechniques 24(5):810-6); 8) colorimetric procedures; and 9) FRET.

Useful information also can be obtained, for example, by performing the assays of the invention with cell extracts. In a specific embodiment, different substrates of an enzymatic kinase reaction are immobilized on the surface of a solid support and the proteins of the cell extract are also immobilized on the surface. The proteins of the cell extract and the substrates of an enzymatic kinase reaction are then incubated with a reaction mixture providing conditions conducive to the occurrence of the enzymatic reaction. The cellular repertoire of particular enzymatic activities can thereby be assessed.

In a more specific embodiment, a plurality of different substrates is immobilized on the surface of the solid support in a well. In specific embodiments, a plurality of wells is present on the microarray and each well contains the plurality of different substrates. The proteins of a cellular extract are also immobilized on the surface of the solid support in wells. Thus, different enzymatic kinase reactions can be tested simultaneously on the microarray. In certain embodiments, the assay of the invention can be performed with whole cells or preparations of plasma membranes. Thus, use of several classes of substrates and reaction buffers can provide for large-scale or exhaustive analysis of cellular activities. In particular, one or several screens can form the basis of identifying a “footprint” of the cell type or physiological state of a cell, tissue, organ or system. For example, different cell types (either morphological or functional) can be differentiated by the pattern of cellular activities or expression determined by the protein chip. This approach also can be used to determine, for example, different stages of the cell cycle, disease states, altered physiologic states (e.g., hypoxia), physiological state before or after treatment (e.g., drug treatment), metabolic state, stage of differentiation or development, response to environmental stimuli (e.g., light, heat), cell-cell interactions, cell-specific gene and/or protein expression, and disease-specific gene and/or protein expression.

In a specific embodiment, compounds that modulate the enzymatic activity of a kinase or kinases on a chip can be identified. For example, changes in the level of enzymatic activity are detected and quantified by incubation of a compound or mixture of compounds with an kinase reaction on the microarray, wherein a signal is produced (e.g., from substrate that becomes fluorescent upon kinase activity). Differences between the presence and absence of the compound are noted. Furthermore, the differences in effects of compounds on enzymatic activities of different kinases are readily detected by comparing their relative effect on samples within the protein chips and between chips.

In certain embodiments, the enzymatic activity detected using a method of the invention is in part due to autocatalysis, i.e., the kinase acts on itself as well as on a substrate. A nonlimiting example of autocatalysis is auto-phosphorylation.

In certain embodiments, immobilizing a substrate and a kinase in proximity sufficient for the occurrence of an enzymatic reaction between the substrate and the kinase induces the catalytic activity of the kinase. In certain embodiments, immobilizing a substrate and a kinase in proximity sufficient for the occurrence of an enzymatic reaction between the substrate and the kinase induces the autocatalytic activity of the kinase.

In certain embodiments, an enzymatic activity is enhanced by immobilizing kinase and substrate in proximity sufficient for the occurrence of the enzymatic reaction. In a specific embodiment, the activity is enhanced compared to the activity in solution.

In certain aspects of the invention, the kinase catalyzes a reaction in which a detectable group is associated with, or dissociated from, a substrate. For example, the detectable group can be a labeled moiety, such as a labeled phosphate group, sugar moiety, polysaccharide, nucleotide, oligonucleotide, amino acid, or peptide.

In certain aspects, a substrate and a kinase are immobilized on a solid support in methods for assaying an enzymatic activity.

Any kinase known to the skilled artisan can be used with the methods of the invention and with protein arrays of the invention. Kinases that can be used with the methods of the invention and immobilization on the microarrays of the invention include but are not limited to those shown in Table 1 and Table 2.

In another aspect the detection step can be detecting a positive signal of phosphorylation in the vicinity of the immobilized substrate. Not to be limited by theory, but the positive signal may come form enhanced autophosphorylation of the kinase or phosphorylation of the substrate.

TABLE 1

Hexokinase, Glucokinase, Ketohexokinase, Fructokinase, Rhamnulokinase,
Galactokinase, Mannokinase, Glucosamine kinase, Phosphoglucokinase, 6-
phosphofructokinase, Gluconokinase, Dehydogluconokinase,
Sedoheptulokinase, Ribokinase, L-ribulokinase, Xylulokinase,
Phosphoribokinase, Phosphoribulokinase, Adenosine kinase, Thymidine
kinase, Ribosylnicotinamide kinase, NAD(+) kinase, Dephospho-CoA kinase,
Adenylylsulfate kinase, Riboflavin kinase, Erythritol kinase,
Triokinase, Glycerone kinase, Glycerol kinase, Glycerate kinase, Choline
kinase, Pantothenate kinase, Pantetheine kinase, Pyridoxal kinase,
Mevalonate kinase, Protein kinase, Phosphorylase kinase, Homoserine
kinase, Pyruvate kinase, Glucose-1-phosphate phosphodismutase,
Riboflavin phosphotransferase, Glucuronokinase, Galacturonokinase, 2-
dehydro-3-deoxygluconokinase, L-arabinokinase, D-ribulokinase, Uridine
kinase, Hydroxymethylpyrimidine kinase, Hydroxyethylthiazole kinase, L-
fuculokinase, Fucokinase, L-xylulokinase, D-arabinokinase, Allose
kinase, 1-phosphofructokinase, 2-dehydro-3-deoxygalactonokinase, N-
acetylglucosamine kinase, N-acylmannosamine kinase, Acyl-phosphate-
hexose phosphotransferase, Phosphoramidate-hexose phosphotransferase,
Polyphosphate-glucose phosphotransferase, Inositol 3-kinase, Scyllo-
inosamine kinase, Undecaprenol kinase, 1-phosphatidylinositol 4-kinase,
1-phosphatidylinositol-4-phosphate 5-kinase, Protein-N(pi)-
phosphohistidine-sugar phosphotransferase, Protamine kinase, Shikimate
kinase, Streptomycin 6-kinase, Inosine kinase, Deoxycytidine kinase,
Deoxyadenosine kinase, Nucleoside phosphotransferase, Polynucleotide 5′-
hydroxyl-kinase, Diphosphate--glycerol phosphotransferase, Diphosphate--
serine phosphotransferase, Hydroxylysine kinase, Ethanolamine kinase,
Pseudouridine kinase, Alkylglycerone kinase, Beta-glucoside kinase, NADH
kinase, Streptomycin 3′′-kinase, Dihydrostreptomycin-6-phosphate 3′-
alpha-kinase, Thiamine kinase, Diphosphate--fructose-6-phosphate 1-
phosphotransferase, Sphinganine kinase, 5-dehydro-2-deoxygluconokinase,
Alkylglycerol kinase, Acylglycerol kinase, Kanamycin kinase, [Pyruvate
dehydrogenase (lipoamide)] kinase, 5-methylthioribose kinase, Tagatose
kinase, Hamamelose kinase, Viomycin kinase, Diphosphate-protein
phosphotransferase, 6-phosphofructo-2-kinase, Glucose-1,6-bisphosphate
synthase, Diacylglycerol kinase, Dolichol kinase,
[Hydroxymethylglutaryl-CoA reductase (NADPH)] kinase, Dephospho-
[reductase kinase] kinase, Protein-tyrosine kinase, Deoxyguanosine
kinase, AMP--thymidine kinase, [3-methyl-2-oxobutanoate dehydrogenase
(lipoamide)] kinase, [Isocitrate dehydrogenase (NADP+)] kinase, [Myosin
light-chain] kinase, ADP--thymidine kinase, Hygromycin-B kinase,
Caldesmon kinase, Phosphoenolpyruvate--glycerone phosphotransferase,
Xylitol kinase, Calcium/calmodulin-dependent protein kinase, Tyrosine 3-
monooxygenase kinase, Rhodopsin kinase, [Beta-adrenergic-receptor]
kinase, Inositol-trisphosphate 3-kinase, [Acetyl-CoA carboxylase]
kinase, [Myosin heavy-chain] kinase, Tetraacyldisaccharide 4′-kinase,
[Low-density lipoprotein receptor] kinase, Tropomyosin kinase, Inositol-
tetrakisphosphate 1-kinase, [Tau protein] kinase, Macrolide 2′-kinase,
Phosphatidylinositol 3-kinase, Ceramide kinase, 1D-myo-inositol-
tetrakisphosphate 5-kinase, [RNA-polymerase]-subunit kinase, Glycerol-3-
phosphate-glucose phosphotransferase, Diphosphate-purine nucleoside
kinase, Tagatose-6-phosphate kinase, Deoxynucleoside kinase, ADP-
specific phosphofructokinase, ADP-specific glucokinase, 4-(cytidine 5′-
diphospho)-2-C-methyl-D-erythritol kinase, 1-phosphatidylinositol-5-
phosphate 4-kinase, 1-phosphatidylinositol-3-phosphate 5-kinase,
Inositol-polyphosphate multikinase, Inositol-hexakisphosphate kinase,
Phosphatidylinositol-4,5-bisphosphate 3-kinase, Phosphatidylinositol-4-
phosphate 3-kinase, Acetate kinase, Carbamate kinase, Phosphoglycerate
kinase, Aspartate kinase, Formate kinase, Butyrate kinase,
Acetylglutamate kinase, Phosphoglycerate kinase (GTP), Glutamate 5-
kinase, Acetate kinase (diphosphate), Glutamate 1-kinase, Branched-
chain-fatty-acid kinase, Guanidoacetate kinase, Creatine kinase,
Arginine kinase, Taurocyamine kinase, Lombricine kinase.
Hypotaurocyamine kinase, Opheline kinase, Ammonia kinase,
Phosphoenolpyruvate--protein phosphatase, Agmatine kinase, Protein-
histidine pros-kinase, Protein-histidine tele-kinase, Polyphosphate
kinase, Phosphomevalonate kinase, Adenylate kinase, Nucleoside-phosphate
kinase, Nucleoside-diphosphate kinase, Phosphomethylpyrimidine kinase,
Guanylate kinase, Thymidylate kinase, Nucleoside-triphosphate--adenylate
kinase, (Deoxy) adenylate kinase, T2-induced deoxynucleotide kinase,
(Deoxy) nucleoside-phosphate kinase, Cytidylate kinase, Thiamine-
diphosphate kinase, Thiamine-phosphate kinase, 3-phosphoglyceroyl-
phosphate-polyphosphate phosphotransferase, Farnesyl-diphosphate kinase,
5-methyldeoxycytidine-5′-phosphate kinase, Dolichyl-diphosphate--
polyphosphate phosphotransferase, Ribose-phosphate pyrophosphokinase,
Thiamine pyrophosphokinase, 2-amino-4-hydroxy-6-
hydroxymethyldihydropteridine pyrophosphokinase, Nucleotide
pyrophosphokinase, GTP pyrophosphokinase, Nicotinamide-nucleotide
adenylyltransferase, FMN adenylyltransferase, Pantetheine-phosphate
adenylyltransferase, Sulfate adenylyltransferase, Sulfate
adenylyltransferase (ADP), DNA-directed RNA polymerase, DNA-directed DNA
polymerase, Polyribonucleotide nucleotidyltransferase, UTP--glucose-1-
phosphate uridylyltransferase, UTP--hexose-1-phosphate
uridylyltransferase, UTP--xylose-1-phosphate uridylyltransferase, UDP-
glucose--hexose-1-phosphate uridylyltransferase, Mannose-1-phosphate
guanylyltransferase, Ethanolamine-phosphate cytidylyltransferase,
Cholinephosphate cytidylyltransferase, Nicotinate-nucleotide
adenylyltransferase, Polynucleotide adenylyltransferase, tRNA
cytidylyltransferase, Mannose-1-phosphate guanylyltransferase (GDP),
UDP-N-acetylglucosamine pyrophosphorylase, Glucose-1-phosphate
thymidylyltransferase, tRNA adenylyltransferase, Glucose-1-phosphate
adenylyltransferase, Nucleoside-triphosphate-hexose-1-phosphate
nucleotidyltransferase, Hexose-1-phosphate guanylyltransferase, Fucose-
1-phosphate guanylyltransferase, DNA nucleotidylexotransferase,
Galactose-1-phosphate thymidylyltransferase, Glucose-1-phosphate
cytidylyltransferase, Glucose-1-phosphate guanylyltransferase, Ribose-5-
phosphate adenylyltransferase, Aldose-1-phosphate adenylyltransferase,
Aldose-1-phosphate nucleotidyltransferase, 3-deoxy-manno-octulosonate
cytidylyltransferase, Glycerol-3-phosphate cytidylyltransferase, D-
ribitol-5-phosphate cytidylyltransferase, Phosphatidate
cytidylyltransferase, Glutamate-ammonia-ligase adenylyltransferase,
Acylneuraminate cytidylyltransferase, Glucuronate-1-phosphate
uridylyltransferase, Guanosine-triphosphate guanylyltransferase,
Gentamicin 2′′-nucleotidyltransferase, Streptomycin 3′′-
adenylyltransferase, RNA-directed RNA polymerase, RNA-directed DNA
polymerase, mRNA guanylyltransferase, Adenylylsulfate--ammonia
adenylyltransferase, RNA uridylyltransferase, ATP adenylyltransferase,
Phenylalanine adenylyltransferase, Anthranilate adenylyltransferase,
tRNA nucleotidyltransferase, N-methylphosphoethanolamine
cytidylyltransferase, (2,3-dihydroxybenzoyl) adenylate synthase,
[Protein-PII] uridylyltransferase, 2-C-methyl-D-erythritol 4-phosphate
cytidylyltransferase, Holo-citrate lyase synthase,
Ethanolaminephosphotransferase, Diacylglycerol
cholinephosphotransferase, Ceramide cholinephosphotransferase, Serine-
phosphoethanolamine synthase , CDP-diacylglycerol--glycerol-3-phosphate
3-phosphatidyltransferase, Undecaprenyl-phosphate
galactosephosphotransferase, Holo-[acyl-carrier protein] synthase, CDP-
diacylglycerol--serine O-phosphatidyltransferase, Phosphomannan
mannosephosphotransferase, Sphingosine cholinephosphotransferase, CDP-
diacylglycerol--inositol 3-phosphatidyltransferase, CDP-glycerol
glycerophosphotransferase, Phospho-N-acetylmuramoyl-pentapeptide-
transferase, CDP-ribitol ribitolphosphotransferase, UDP-N-
acetylglucosamine--dolichyl-phosphate N-
acetylglucosaminephosphotransferase, UDP-N-acetylglucosamine--lysosomal-
enzyme N-acetylglucosaminephosphotransferase, UDP-galactose--UDP-N-
acetylglucosamine galactosephosphotransferase, UDP-glucose--glycoprotein
glucosephosphotransferase, Phosphatidylglycerol--membrane-
oligosaccharide glycerophosphotransferase, Membrane-oligosaccharide
glycerophosphotransferase, 1-alkenyl-2-acylglycerol
cholinephosphotransferase, Carboxyvinyl-carboxyphosphonate
phosphorylmutase, Phosphatidylcholine synthase, Triphosphoribosyl-
dephospho-CoA synthase, Pyruvate, phosphate dikinase, Pyruvate, water
dikinase, Selenide, water dikinase, Alpha-glucan, water dikinase, Protein
kinase C, Phosphoenolpyruvate carboxykinase (GTP), Phosphoenolpyruvate
carboxykinase (pyrophosphate), Phosphoenolpyruvate carboxykinase (ATP),

Identifying substrates of enzymes can also be conducted with the methods of the present invention. For example, a wide variety of different potential substrates are attached to the protein chip and are assayed for their ability to act as a substrate for particular kinase(s) that is also immobilized to the surface of the solid substrate.

In certain embodiments, candidate-substrates are identified in a parallel experiment on the basis of a substrates' ability to bind to the kinase of interest. A substrate can be a cell, protein-containing cellular material, protein, oligonucleotide, polynucleotide, DNA, RNA, small molecule substrate, drug candidate, receptor, antigen, steroid, phospholipid, antibody, immunoglobulin domain, glutathione, maltose, nickel, dihydrotrypsin, or biotin. After incubation of proteins on a chip with test molecules, the candidate substrates can be identified by mass spectrometry (Lakey et al., 1998, “Measuring protein-protein interactions”, Curr Opin Struct Biol. 8:119-23).

The identity of targets of a specific enzymatic activity can be assayed by treating a protein chip with complex protein mixtures, such as cell extracts, and determining protein activity, wherein the complex protein mixture is also immobilized on the surface of the solid support. For example, a protein chip containing an array of different kinases can be contacted with a cell extract from cells treated with a compound (e.g., a drug), and assayed for kinase activity. In another example, a protein chip containing an array of different kinases can be contacted with a cell extract from cells at a particular stage of cell differentiation (e.g., pluripotent) or from cells in a particular metabolic state (e.g., mitotic), and assayed for kinase activity. Proteins of the cell extract can be immobilized to the solid support by methods as described above. The results obtained from such assays, comparing for example, cells in the presence or absence of a drug, or cells at several differentiation stages, or cells in different metabolic states, can provide information regarding the physiologic changes in the cells between the different conditions.

Alternatively, the identity of targets of specific cellular activities can be assayed by treating a protein chip, containing many different proteins (e.g., a peptide library) immobilized to the surface of the solid support of the protein chip, with a complex protein mixture (e.g., such as a cell extract), and assaying for modifications to the proteins on the chip, wherein the protein mixture is also immobilized to the surface of the solid support. For example, a protein chip containing an array of different proteins can be contacted with a cell extract from cells treated with a compound (e.g., a drug), and assayed for kinase or other transferase activity can for example be assayed. In another example, a protein chip containing an array of different proteins can be contacted with a cell extract from cells at a particular stage of cell differentiation (e.g., pluripotent) or from cells in a particular metabolic state (e.g., mitotic). The results obtained from such assays, comparing for example, cells in the presence or absence of a drug, or cells at several stages of differentiation, or cells in different metabolic states, can provide information regarding the physiologic effect on the cells under these conditions.

The activity of proteins exhibiting differences in function, such as enzymatic activity, can be analyzed with the protein methods of the present invention. For example, differences in protein isoforms derived from different alleles are assayed for their activities relative to one another.

The methods of the invention can be used for drug discovery, analysis of the mode of action of a drug, drug specificity, and prediction of drug toxicity. As many kinases and substrates can be tested at the same time, the methods of the invention are suitable to determine profiles for different drugs. In certain embodiments, such a profile relates to sensitivities of different kinases to the drug of interest. In other embodiments, such a profile relates to effects of the drug of interest on the substrate specificity of different kinases. For example, the identity of kinases whose activity is susceptible to a particular compound can be determined by performing the assay of the invention in the presence and absence of a compound. More detailed description of screening assays using the methods of the invention are described herein

Moreover, the methods of the present invention can be used to determine the presence of potential inhibitors, catalysts, modulators, or enhancers of kinase activity. In one example, a cellular extract of a cell is added to an kinase assay of the invention.

The protein chips of the invention can be used to determine the effects of a drug on the modification of multiple targets by complex protein mixtures, such as for example, whole cells, cell extracts, or tissue homogenates. The net effect of a drug can be analyzed by screening one or more protein chips with drug-treated cells, tissues, or extracts, which then can provide a “signature” for the drug-treated state, and when compared with the “signature” of the untreated state, can be of predictive value with respect to, for example, potency, toxicity, and side effects. Furthermore, time-dependent effects of a drug can be assayed by, for example, adding the drug to the cell, cell extract, tissue homogenate, or whole organism, and applying the drug-treated cells or extracts to a protein chip at various timepoints of the treatment.

Exemplary kinase assays for use with the invention are described below. These examples are meant to illustrate the present invention and are not intended to limit in any way the scope of the present invention.

### Kinase Assay

In certain embodiments of the invention, the enzymatic reaction to be performed with the methods of the invention is a kinase reaction. In certain embodiments, a kinase is a tyrosine kinase or a serine/threonine kinase. Exemplary kinases to be used with the methods of the invention include, but not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44 mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). Further exemplary kinases are listed in Table 2 below and Table 1 above. In addition, a recent list of human kinases can be found in Manning et al., 2002, Science 298:1912-1934. In certain embodiments of the invention, proteins to be used with the methods of the invention and on the arrays of the invention are proteins that have sequence homologies to a known kinase.

TABLE 2

| | | | | | | |
|---|---|---|---|---|---|---|
| Description | Aliase(s) | Cat. No. | Accession Number | Length | Expression | Tag |

Serine/Threonine Protein Kinases

| | | | | | | |
|---|---|---|---|---|---|---|
| ADRBK1 | GRK2 | PV3361 | AAH37963, | Full | Insect | C-terminal His |
| | | | NP_001610 | | | |
| ADRBK2 | GRK3 | PV3827 | NP_005151, V308M | Full | Insect | N-terminal GST |
| AKT1 | PKB alpha | P2999 | NP_005154 | Full | Insect | N-terminal His |
| AKT2 | PKB beta | PV3184 | NP_001617 | Full | Insect | N-terminal His |
| AKT3 | PKB | PV3185 | NP_005456 | Full | Insect | N-terminal His |
| | gamma | | | | | |
| AURKB | Aurora B | PV3970 | NP_004208 | Full | Insect | N-terminal His |
| AURKC | Aurora C | PV3856 | AAH75064 | Full Catalytic | Insect | N-terminal GST |
| | | | | (15-289) | | |
| BRAF | | PV3848 | NP_004324.2 | Full | Insect | N-terminal GST |
| BRAF V599E | | PV3849 | NP_004324.1, | Full Catalytic | Insect | N-terminal GST |
| | | | V599E | (416-766) | | |
| CAMK1D | CaMKI | PV3663 | NP_705718 | Full | Insect | N-terminal His |
| | delta | | | | | |
| CAMK2A | CaMKII | PV3142 | NP_037052 | Full | Insect | C-terminal His |
| | alpha | | | | | |
| CAMK2D | CaMKII | PV3373 | NP_742113 | Full | Insect | C-terminal His |
| | delta | | | | | |
| CAMK4 | CaMKIV | PV3310 | NP_001735 | Full | E. coli | N-terminal GST |
| CDK1/cyclin | | PV3292 | NP_001777, | Full | Insect | C-terminal His |
| B | | | NP_114172 | | | |
| CDK2/cyclin | | PV3267 | NP_001789, | Full | Insect | N-terminal His |
| A | | | NP_001228 | | | |
| CDK5/p35 | | PV3000 | NP_004926.1 | Full | Insect | N-terminal His |
| CDK7/cyclin | | PV3868 | NP_001790 | Full | Insect | N-terminal His |
| H/MNAT1 | | | (Q130R), | | | |
| | | | NP_001230, | | | |
| | | | NP_002422) | | | |
| CHEK1 | CHK1 | P3040 | NP_001265 | Full | Insect | N-terminal His |
| CHEK2 | CHK2 | PV3367 | NP_009125 | Full | Insect | C-terminal His |
| CLK1 | | PV3315 | NP_004062 | Full Catalytic | E. coli | N-terminal GST |
| CLK3 | | PV3826 | NP_003983, T132S, | Full | Insect | N-terminal GST |
| | | | G133S | | | |
| CLK4 | | PV3839 | NP_065717 | Full | Insect | N-terminal GST |
| CSNK1A1 | CK1 | PV3850 | NP_001883, K164Q | Full | Insect | N-terminal GST |
| CSNK1D | CK1 delta | PV3665 | NP_620693 | Full | Insect | N-terminal GST |
| CSNK1E | CK1 epsilon | PV3500 | NP_001885 | Full | Insect | C-terminal His |
| CSNK1G1 | CK1 | PV3825 | NP_071331 | Full | Insect | N-terminal GST |
| | gamma 1 | | | | | |
| CSNK1G2 | CK1 | PV3499 | NP_001310 | Full | Insect | C-terminal His |
| | gamma 2 | | | | | |
| CSNK1G3 | CK1 | PV3838 | NP_004375, R174G | Full | Insect | N-terminal GST |
| | gamma 3 | | | | | |
| CSNK2A1 | CK2 alpha | PV3248 | NP_001886 | Full | Insect | C-terminal His |
| | 1 | | | | | |
| CSNK2A2 | CK2 alpha | PV3624 | NP_001887 | Full | Insect | N-terminal GST |
| | 2 | | | | | |
| DAPK1 | | PV3969 | NP_004929 | Full Catalytic | Insect | N-terminal GST |
| | | | | (1-363) | | |
| DAPK2 | | PV3614 | NP_055141 | Full | Insect | N-terminal GST |
| DAPK3 | ZIPK | PV3686 | NP_001339 | Full | Insect | N-terminal GST |
| DMPK | | PV3784 | NP_004400 | Full | Insect | N-terminal GST |
| DYRK1A | | PV3785 | NP_001387 | Full | Insect | N-terminal GST |
| DYRK3 | | PV3837 | NP_003573 | Full | Insect | N-terminal GST |
| DYRK4 | | PV3871 | NP_003836.1 | Full | Insect | N-terminal GST |
| HIPK4 | | PV3852 | NP_653286 | Full | Insect | N-terminal GST |
| GRK4 | | PV3807 | NP_892027 | Full | Insect | N-terminal GST |
| GRK5 | | PV3824 | NP_005299 | Full | Insect | N-terminal GST |
| GRK6 | | PV3661 | NP_001004106 | Full | Insect | N-terminal GST |
| GRK7 | | PV3823 | NP_631948 | Full | Insect | N-terminal GST |
| GSK3A | GSK3 | PV3270 | NP_063937 | Full | Insect | C-terminal His |
| | alpha | | | | | |
| GSK3B | GSK3 beta | PV3365 | NP_002084 | Full | Insect | C-terminal His |
| IKBKB | IKK beta | PV3836 | NP_001547 | Full | Insect | N-terminal GST |
| IRAK4 | | PV3362 | NP_057207, | Full | Insect | N-terminal His |
| | | | AAH13316 | | | |
| MAP2K1 | MEK1 | PV3303 | NP_002746 | Full | Insect | N-terminal His |
| MAP2K1, | MEK1 | P3099 | NP_002746, S218D, | Full | Insect | N-terminal His |
| mutant | | | S222D | | | |
| MAP2K2 | MEK2 | PV3615 | NP_109587 | Full | Insect | C-terminal His |
| MAP2K3 | MEK3 | PV3662 | NP_002747 | Full | Insect | N-terminal GST |
| MAP2K6 | MKK6 | PV3318 | NP_002749 | Full | Insect | N-terminal His |
| MAP2K6, | MKK6 | PV3293 | NP_002749, S207E, | Full | Insect | N-terminal His |
| mutant | | | T211E | | | |
| MAP3K11 | MLK3 | PV3788 | NP_002410.1 | Full | Insect | N-terminal GST |
| MAP3K2 | MEKK2 | PV3822 | AAF63496.1 | Full | Insect | N-terminal GST |
| MAP3K3 | MEKK3 | PV3876 | NP_002392 | Full | Insect | N-terminal GST |
| MAP3K5 | ASK1 | PV3809 | NP_005914 | Full | Insect | N-terminal GST |
| MAP3K9 | MLK1 | PV3787 | NP_149132 | Full Catalytic | Insect | N-terminal GST |
| | | | | (1-500) | | |
| MAP3K10 | MLK2 | PV3877 | NP_002437 | Full | Insect | N-terminal GST |
| MAP4K4 | HGK | PV3687 | NP_004824 | Full Catalytic | Insect | N-terminal GST |
| | | | | (1-328) | | |
| MAP4K5 | KHS1 | PV3682 | NP_942089 | Full | Insect | N-terminal GST |
| MAPK1 | ERK2 | PV3313 | NP_620407 | Full | E. coli | N-terminal GST |
| MAPK11 | p38 beta | PV3679 | NP_002742 | Full | Insect | N-terminal His |
| MAPK12 | p38 gamma | PV3654 | NP_002960 | Full | Insect | N-terminal His |
| MAPK13 | p38 delta | PV3656 | NP_002745 | Full | Insect | N-terminal His |
| MAPK14 | p38 alpha | PV3304 | NP_620581 | Full | E. coli | N-terminal GST |
| MAPK3 | ERK1 | PV3311 | NP_002737 | Full | E. coli | N-terminal GST |
| MAPK8 | JNK1 | PV3319 | NP_002741.1 | Full | Insect | N-terminal His |
| MAPK9 | JNK2 | PV3620 | NP_002743 | Full | Insect | N-terminal His |
| MAPKAPK2 | | PV3317 | NP_116584 | Full | E. coli | N-terminal His |
| MAPKAPK3 | | PV3299 | NP_004626 | Full | Insect | N-terminal His |
| MAPKAPK5 | PRAK | PV3301 | NP_003659 | Full | Insect | N-terminal His |
| MARK2 | | PV3878 | NP_059672, Q592P, | Full | Insect | N-terminal GST |
| | | | F357S | | | |
| MARK4 | | PV3851 | NP_113605 | Full | Insect | N-terminal GST |
| MINK1 | | PV3810 | NP_056531 | Full Catalytic | Insect | N-terminal GST |
| | | | | (1-320) | | |
| MLCK | MLCK2 | PV3835 | NP_872299, R175G | Full | Insect | N-terminal GST |
| MST4 | | PV3690 | NP_057626.2 | Full | Insect | N-terminal GST |
| MYLK2 | skMLCK | PV3757 | NP_149109 | Full | Insect | N-terminal GST |
| NEK2 | | PV3360 | NP_002488 | Full | Insect | C-terminal His |
| NEK3 | | PV3821 | NP_689933 | Full | Insect | N-terminal GST |
| NEK6 | | PV3353 | NP_055212 | Full Catalytic | Insect | C-terminal His |
| NEK7 | | PV3833 | NP_598001.1 | Full | Insect | N-terminal GST |
| PAK1 | | PV3820 | NP_002567 | Full | Insect | N-terminal GST |
| PAK3 | | PV3789 | NP_002569 | Full | Insect | N-terminal His |
| PAK4 | | PV3845 | NP_005875 | Full | Insect | N-terminal GST |
| PAK6 | | PV3502 | NP_064553 | Full | Insect | C-terminal His |
| PASK | | PV3972 | NP_055963 | Full Catalytic | Insect | N-terminal GST |
| | | | | (879-1323) | | |
| PDK1 | | P3001 | NP_002604 | Full | Insect | N-terminal His |
| PHKG1 | | PV3853 | NP_006204 | Full | Insect | N-terminal GST |
| PHKG2 | | PV3369 | NP_000285 | Full | Insect | C-terminal His |
| PIM1 | | PV3503 | NP_002639 | Full | Insect | C-terminal His |
| PIM2 | | PV3649 | NP_006866 | Full | Insect | N-terminal GST |
| PKN1 | PRK1 | PV3790 | NP_998725 | Full | Insect | N-terminal GST |
| PKN2 | PRK2 | PV3879 | NP_006247 | Full | Insect | N-terminal GST |
| PLK1 | | PV3501 | NP_005021 | Full | Insect | none |
| PLK3 | | PV3812 | NP_004064 | Full Catalytic | Insect | N-terminal GST |
| | | | | (58-340) | | |
| PRKACA | PKA | P2912 | NP_002721.1 | Full Catalytic | E. coli | N-terminal His |
| PRKCA | PKC alpha | P2232, | NP_002728 | Full | Insect | none |
| | | P2227 | | | | |
| PRKCB1 | PKC beta I | P2291, | NP_997700.1 | Full | Insect | none |
| | | P2281 | | | | |
| PRKCB2 | PKC beta II | P2254, | NP_002729 | Full | Insect | none |
| | | P2251 | | | | |
| PRKCD | PKC delta | P2293, | NP_006245 | Full | Insect | none |
| | | P2287 | | | | |
| PRKCE | PKC | P2292, | NP_005391.1 | Full | Insect | none |
| | epsilon | P2282 | | | | |
| PRKCG | PKC | P2233, | NP_002730 | Full | Insect | none |
| | gamma | P2228 | | | | |
| PRKCH | PKC eta | P2633, | NP_006246 | Full | Insect | N-terminal His |
| | | P2634 | | | | |
| PRKCI | PKC iota | PV3183, | NP_002731 | Full | Insect | N-terminal His |
| | | PV3186 | | | | |
| PRKCQ | PKC theta | P2996 | NP_006248 | Full | Insect | C-terminal His |
| PRKCZ | PKC zeta | P2273, | NP_002735 | Full | Insect | none |
| | | P2268 | | | | |
| PKC Isozyme | PKC | P2352 | | Full | Insect | n/a |
| Panel | Isozyme | | | | | |
| | Panel | | | | | |
| PRKD1 | PKD, PKC | PV3791 | NP_002733 | Full | Insect | N-terminal GST |
| | mu | | | | | |
| PRKD2 | PKD2 | PV3758 | NP_057541 | Full | Insect | N-terminal GST |
| PRKD2 | PKD2 | PV3352 | NP_057541 | Full Catalytic | Insect | N-terminal His |
| PRKCN | PKD3 | PV3692 | NP_005084 | Full | Insect | N-terminal GST |
| PRKG2 | PKG2 | PV3973 | NP_006250 | Full | Insect | N-terminal GST |
| PRKX | | PV3813 | NP_005035 | Full | Insect | N-terminal GST |
| RAF1 | cRAF | PV3805 | NP_002871, Y340D, | Full Catalytic | Insect | N-terminal GST |
| | | | Y341D | (306-648) | | |
| ROCK1 | | PV3691 | NP_005397 | Full Catalytic | Insect | N-terminal GST |
| | | | | (1-535) | | |
| ROCK2 | | PV3759 | NP_004841 | Full Catalytic | Insect | N-terminal GST |
| | | | | (1-552) | | |
| RPS6KA1 | RSK1 | PV3680 | NP_002944 | Full | Insect | N-terminal His |
| RPS6KA2 | RSK3 | PV3846 | NP_066958 | Full | Insect | N-terminal His |
| RPS6KA3 | RSK2 | PV3323 | NP_004577 | Full | Insect | C-terminal His |
| RPS6KA4 | MSK2 | PV3782 | NP_003933 | Full | Insect | N-terminal GST |
| RPS6KA5 | MSK1 | PV3681 | NP_004746.2 | Full | Insect | N-terminal GST |
| RPS6KB1 | p70S6K | PV3815 | NP_003152, T412E | Full Catalytic | Insect | N-terminal GST |
| | | | | (1-421) | | |
| RPS6KB2 | p70S6K | PV3831 | NP_003943 | Full | Insect | N-terminal GST |
| | beta | | | | | |
| SGK | SGK1 | PV3818 | NP_005618, S589D | Full Catalytic | Insect | N-terminal GST |
| | | | | (60-431) | | |
| SGK2 | | PV3858 | NP_057360, S416D | Full Catalytic | Insect | N-terminal GST |
| | | | | (54-427) | | |
| SGKL | SGK3 | PV3859 | NP_037389, S487D | Full Catalytic | Insect | N-terminal GST |
| | | | | (87-496) | | |
| SLK | | PV3830 | NP_055535.1 | Full | Insect | N-terminal GST |
| SRPK2 | | PV3829 | NP_872633 | Full | Insect | N-terminal GST |
| STK3 | MST2 | PV3684 | NP_006272 | Full | Insect | N-terminal His |
| STK4 | MST1 | PV3854 | NP_006273 | Full | Insect | N-terminal GST |
| STK6 | Aurora A | PV3612 | NP_940839 | Full | Insect | N-terminal His |
| STK17A | DRAK1 | PV3783 | NP_004751 | Full | Insect | N-terminal GST |
| STK22B | TSSK2 | PV3622 | NP_443732 | Full | Insect | N-terminal His |
| STK22D | TSSK1 | PV3505 | NP_114417 | Full | Insect | C-terminal His |
| STK23 | MSSK1 | PV3880 | NP_055185 | Full | Insect | N-terminal GST |
| STK24 | MST3 | PV3650 | NP_003567 | Full | Insect | N-terminal GST |
| STK25 | YSK1 | PV3657 | NP_006365 | Full | Insect | N-terminal GST |
| STK31 | SgK396 | PV3862 | NP_113602 | Full | Insect | N-terminal GST |
| TAOK2 | TAO1 | PV3760 | NP_004774 | Full Catalytic | Insect | N-terminal GST |
| | | | | (1-314) | | |
| TAOK3 | JIK | PV3652 | NP_057365 | Full | Insect | N-terminal GST |
| TBK1 | | PV3504 | NP_037386 | Full | Insect | N-terminal His |
| TTK | | PV3792 | NP_003309 | Full | Insect | N-terminal GST |
| WEE1 | | PV3817 | NP_003381.1 | Full | Insect | N-terminal GST |
| ZAK | | PV3882 | NP_598407 | Full | Insect | N-terminal GST |

Cytoplasmic Tyrosine Protein Kinases

| | | | | | | |
|---|---|---|---|---|---|---|
| ABL1 | | P3049 | AAB60934, | Full | Insect | C-terminal His |
| | | | NP_005148 | | | |
| ABL1 | | PV3863 | AAB60934 | Full | Insect | C-terminal His |
| Y253F | | | | | | |
| ABL1 | | PV3864 | AAB60934 | Full | Insect | C-terminal His |
| E255K | | | | | | |
| ABL1 | | PV3865 | AAB60934 | Full | Insect | C-terminal His |
| G250E | | | | | | |
| ABL1 T315I | | PV3866 | AAB60934 | Full | Insect | C-terminal His |
| ABL2 | Arg | PV3266 | NP_009298 | Full | Insect | C-terminal His |
| BLK | | PV3683 | NP_001706 | Full | Insect | N-terminal His |
| BMX | | PV3371 | NP_001712 | Full | Insect | C-terminal His |
| BTK | | PV3363 | NP_000052 | Full | Insect | C-terminal His |
| CSK | | P2927 | NP_004374 | Full | E. coli | C-terminal His |
| FER | | PV3806 | NP_005237 | Catalytic | Insect | N-terminal GST |
| | | | | (541-822) | | |
| FES | Fps | PV3354 | NP_001996 | Full | Insect | C-terminal His |
| FGR | | P3041 | NP_005239 | Full | Insect | C-terminal His |
| FRK | PTK5 | PV3874 | NP_002022 | Full | Insect | N-terminal GST |
| FYN | | P3042 | NP_694592 | Full | Insect | C-terminal His |
| HCK | | P2908 | NP_002101 | Full | Insect | C-terminal His |
| ITK | | PV3875 | NP_005537 | Full | Insect | N-terminal GST |
| JAK3 | | PV3855 | NP_000206 | Catalytic | Insect | N-terminal GST |
| | | | | (781-1124) | | |
| LCK | | P3043 | NP_005347 | Full | Insect | C-terminal His |
| LYNA | | P2906 | NP_002341 | Full | Insect | C-terminal His |
| LYNB | | P2907 | AAH59394 | Full | Insect | C-terminal His |
| MATK | Hyl | PV3370 | NP_647611 | Full | Insect | C-terminal His |
| PTK2 | FAK | PV3832 | NP_722560 | Full | Insect | N-terminal GST |
| PTK6 | Brk | PV3291 | NP_005966 | Full | Insect | C-terminal His |
| SRC | | P3044 | NP_005408 | Full | Insect | C-terminal His |
| SRCN1 | | P2904 | NP_005408 | Full | Insect | none |
| SRCN2 | | P2909 | NP_005408 | Full | Insect | none |
| SYK | | PV3857 | NP_003168 | Full | Insect | N-terminal GST |
| TEC | | PV3269 | NP_003206 | Full | Insect | C-terminal His |
| YES1 | Yes | P3078 | NP_005424 | Full | Insect | C-terminal His |
| ZAP70 | | P2782 | NP_001070 | Full | Insect | C-terminal His |

Receptor Tyrosine Protein Kinases

| | | | | | | |
|---|---|---|---|---|---|---|
| ALK | | PV3867 | NP_004295, | Cytoplasmic | Insect | N-terminal GST |
| | | | I1461C | | | |
| AXL | | PV3971 | NP_058713 | Cytoplasmic | Insect | C-terminal His |
| CSF1R | FMS | PV3249 | NP_005202 | Cytoplasmic | Insect | C-terminal His |
| DDR2 | | PV3870 | NP_006173, | Cytoplasmic | Insect | N-terminal GST |
| | | | S642A | | | |
| EGFR | | P2628 | N/A (cell lysate) | Full | A431 Cells | none |
| EGFR | ErbB1 | PV3872 | NP_005219.2 | Cytoplasmic | Insect | N-terminal GST |
| EGFR | ErbB1 | PV3873 | NP_005219.2, | Cytoplasmic | Insect | N-terminal GST |
| L861Q | L861Q | | L861Q | | | |
| EPHA1 | | PV3841 | NP_005223.2 | Cytoplasmic | Insect | N-terminal GST |
| EPHA2 | | PV3688 | NP_004422.2 | Cytoplasmic | Insect | N-terminal GST |
| EPHA3 | | PV3359 | NP_005224 | Cytoplasmic | Insect | C-terminal His |
| EPHA4 | | PV3651 | NP_004429.1 | Cytoplasmic | Insect | N-terminal GST |
| EPHA5 | | PV3840 | NP_004430.1 | Cytoplasmic | Insect | N-terminal GST |
| EPHA7 | | PV3689 | NP_004431.1 | Cytoplasmic | Insect | N-terminal GST |
| EPHA8 | | PV3844 | NP_065387 | Cytoplasmic | Insect | N-terminal GST |
| EPHB1 | | PV3786 | NP_004432 | Cytoplasmic | Insect | N-terminal GST |
| EPHB2 | | PV3625 | NP_004433 | Cytoplasmic | Insect | N-terminal GST |
| EPHB3 | | PV3658 | NP_004434 | Cytoplasmic | Insect | N-terminal GST |
| EPHB4 | | PV3251 | NP_004435 | Cytoplasmic | Insect | C-terminal His |
| ERBB2 | HER2 | PV3366 | NP_004439 | Cytoplasmic | Insect | C-terminal His |
| ERBB4 | HER4 | PV3626 | NP_005226 | Cytoplasmic | Insect | N-terminal GST |
| FGFR1 | | PV3146 | NP_000595 | Cytoplasmic | Insect | C-terminal His |
| FGFR2 | | PV3368 | NP_075420 | Cytoplasmic | Insect | C-terminal His |
| FGFR3 | | PV3145 | NP_000133 | Cytoplasmic | Insect | N-terminal His |
| FGFR4 | | P3054 | NP_002002 | Cytoplasmic | Insect | N-terminal His |
| FLT1 | VEGFR1 | PV3666 | NP_002010 | Cytoplasmic | Insect | N-terminal GST |
| FLT3 | | PV3182 | NP_004110 | Cytoplasmic | Insect | C-terminal His |
| FLT3 | | PV3967 | NP_004110 | Cytoplasmic | Insect | C-terminal His |
| D835Y | | | | | | |
| IGF1R | | PV3250 | NP_000866 | Cytoplasmic | Insect | C-terminal His |
| INSR | | PV3664 | NP_000199 | Cytoplasmic | Insect | N-terminal His |
| INSR | | PV3781 | NP_000199 | Cytoplasmic | Insect | N-terminal GST |
| INSRR | IRR | PV3808 | NP_055030 | Cytoplasmic | Insect | N-terminal GST |
| KDR | VEGFR2 | PV3660 | NP_002244 | Cytoplasmic | Insect | C-terminal His |
| KIT | cKit | P3081 | NP_000213 | Cytoplasmic | Insect | N-terminal His |
| KIT T670I | | PV3869 | NP_000213 | Cytoplasmic | Insect | N-terminal His |
| MERTK | cMER | PV3627 | NP_006334 | Cytoplasmic | Insect | N-terminal GST |
| MET | cMet | PV3143 | NP_000236 | Cytoplasmic | Insect | N-terminal His |
| MET | | PV3968 | NP_00236.2 | Cytoplasmic | Insect | N-terminal His |
| M1250T | | | | | | |
| MUSK | | PV3834 | NP_005583.1 | Cytoplasmic | Insect | N-terminal GST |
| NTRK1 | TRKA | PV3144 | NP_002520 | Cytoplasmic | Insect | C-terminal His |
| NTRK2 | TRKB | PV3616 | NP_006171 | Cytoplasmic | Insect | C-terminal His |
| NTRK3 | TRKC | PV3617 | NP_002521 | Cytoplasmic | Insect | C-terminal His |
| PDGFRA | PDGFR | PV3811 | NP_006197 | Cytoplasmic | Insect | N-terminal GST |
| | alpha | | | | | |
| PDGFRA | | PV3847 | NP_006197, | Cytoplasmic | Insect | N-terminal GST |
| T674I | | | T674I | | | |
| PDGFRB | PDGFR beta | P3082 | NP_002600 | Cytoplasmic | Insect | N-terminal His |
| RET | | PV3819 | NP_066124 | Cytoplasmic | Insect | N-terminal GST |
| ROR2 | | PV3861 | NP_004551 | Cytoplasmic | Insect | N-terminal GST |
| ROS1 | | PV3814 | NP_002935 | Cytoplasmic | Insect | N-terminal GST |
| TEK | Tie2 | PV3628 | NP_000450 | Cytoplasmic | Insect | N-terminal GST |
| TYRO3 | RSE | PV3828 | NP_006284 | Cytoplasmic | Insect | N-terminal GST |

In certain embodiments, the plurality of kinases including a tyrosine kinase, and a kinase substrate that is or includes MBP or a derivative or fragment thereof that includes at least 10, 15, 20, or 25 amino acids of MBP that includes a residue that is phosphorylated, are immobilized on the surface of the solid support. In certain embodiments, a tyrosine kinase and a plurality of different substrates that include MBP or a derivative or fragment thereof that includes at least 10, 15, 20, or 25 amino acids of MBP that includes a residue that is phosphorylated are immobilized on the surface of the solid support. In a specific embodiment, at least one substrate is a universal substrate that includes MBP or a derivative or fragment thereof that includes at least 10, 15, 20, or 25 amino acids of MBP that includes a residue that is phosphorylated.

The kinase reaction can be visualized and quantified by any method known to the skilled artisan. In specific embodiments, to visualize the kinase reaction, ATP whose gamma-phosphate is detectably labeled is added to the microarray in a reaction buffer. The reaction buffer provides, in addition to ATP, reaction conditions conducive to the kinase reaction. Reaction conditions include, but are not limited to, pH, salt concentration, concentration of Mg⁺⁺, and detergent concentration. After incubation in the reaction buffer, the microarray is washed to remove any labeled ATP and the product is quantified via the detectably labeled phosphate that has been transferred during the kinase reaction from ATP to the substrate. The signal intensity is proportional to the amount of labeled phosphate on the substrate and thus to the activity of the kinase reaction.

The gamma phosphate of ATP can be detectably labeled by any method known to the skilled artisan. In certain embodiments, the gamma phosphate of ATP is labeled with radioactive phosphorus, such as, but not limited to, ³²P or ³³P. ³⁵S-gamma-ATP can also be used with the methods of the invention. If the phosphate is labeled radioactively, the signal intensity can be evaluated using autoradiography.

Without being bound by theory, some kinases act on a substrate only in a particular molecular context. Such a molecular context may, e.g., consist of certain scaffold proteins. In certain embodiments of the invention, such scaffold proteins are provided with the reaction buffer. In other embodiments, the scaffold proteins are also immobilized on the surface of the solid support.

In certain embodiments, a kinase reaction can be visualized and quantified using antibodies that bind specifically to phosphorylated proteins or peptides. Such antibodies include, but are not limited to antibodies that bind to phospho-serine or antibodies that bind to phospho-tyrosine. The antibody that binds to the phosphorylated protein or peptide can be directly labeled and detected by any method known to the skilled artisan. In other embodiments, a secondary antibody is used to detect the antibody that is bound to the phosphorylated protein or peptide. The more active the kinase reaction is the more antibody will be bound and the stronger the signal will be.

In certain embodiments, phosphorylation can be detected using a molecule that binds to phosphate and that is linked to a detectable label such as, but not limited to, a dye. In preferred embodiments, the dye comprises a metal-chelating moiety. In a specific embodiment, a phosphorylated protein or peptide is detected using a metal-chelating dye such as provided in Pro-Q Diamond stain, a dye available from Molecular Probes. Suitable illustrative ProQ stains include the gel or microarray stain with the microarray stain being preferred.

In an illustrative embodiment, a phosphorylated protein or peptide is detected using a dye containing a metal-chelating moiety. Suitable metal-chelating moieties are moieties characterized as being capable of simultaneously binding metal ions that have affinity for exposed phosphate groups on target molecules, wherein a ternary complex is formed between the metal-chelating moiety, the metal ion and the phosphorylated target molecule. Metal ions that have been found to bind phosphate groups include, without limitation, trivalent gallium, iron and aluminum. Metal-chelating moieties that bind these ions, under certain conditions, include, without limitation, BAPTA, IDA, DTPA and phenanthrolines. Thus, the metal-chelating moieties must 1) bind metal ions that have affinity for phosphate groups, 2) not interfere with the binding of the metal ion for the phosphate groups and 3) maintain a stable ternary complex. Exemplary metal-chelating moieties that fit these three criteria include BAPTA, IDA, DTPA and phenanthrolines.

BAPTA, as used herein, refers to analogs, including fluorescent and nonfluorescent derivatives, of the metal-chelating moiety (1,2-bis(2-aminophenoxy)ethane-N,N,N′,N′-tetraacetic acid) and salts thereof including any corresponding compounds disclosed in U.S. Pat. Nos. 4,603,209; 4,849,362; 5,049,673; 5,453,517; 5,459,276; 5,516,911; 5,501,980; and 5,773,227. These BAPTA-based metal-chelating moieties are well known to those skilled in the art, primarily as calcium indicators due to their ability to bind divalent calcium ions under physiological conditions, i.e. a pH of about 7 and free calcium ion concentrations near the micromolar and submicromolar range. As calcium indicators these compounds are typically used in live cells wherein the indicators are derivatized on a carboxylic group to comprise at least one lipophilic group or specifically an acetoxymethyl (AM) ester group, wherein AM ester is represented as —CH₂OCOCH₃, to produce cell permeant derivatives of the indicators. It is an aspect of the present invention that certain novel compounds can also comprise an ester substrate, such as —CH₂OCOCH₃.

For the sake of clarity the following structure represents preferred present BAPTA metal-chelating moieties having Formula I:

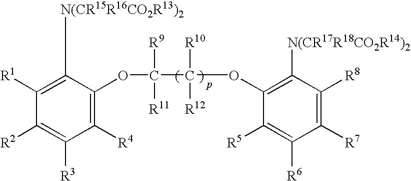

Preferably the two rings are linked by a hydrocarbon bridge between two oxygen atoms in which p is 0, 1 or 2 and the ring substituents (R¹-R⁸) are selected independently from the group consisting of hydrogen, halogen, hydroxyl, alkoxy, alicyclic, heteroalicyclic, alkyl, aryl, amino, aldehyde, carboxyl, nitro, cyano, thioether, sulfinyl, —CH₂OCOCH₃ and linker (L). Typically, the linker comprises a terminal label, reactive group or carrier molecule such as a synthetic polymer or matrix. Alternatively, two adjacent ring substituents in combination constitute a cyclic substituent that is cycloalkyl, cycloheteroalkyl, aryl, fused aryl, heteroaryl or fused heteroaryl. Preferably, the BAPTA metal-chelating moieties have at least two substituents that are not hydrogen, a most preferred BAPTA metal-chelating moiety is substituted by a fluorine atom as one of the substituents, most preferably substituted at the R⁶ or R³ position (e.g., Compounds 1, 2, 5, 7, 8 and 12). Typically the linker attaching the chemical moiety to the BAPTA is at the R², R³, R⁶, or R⁷ position. Equally preferred are BAPTA metal-chelating moieties that comprise a carbonyl group as a substituent, preferably at the R⁷ position, e.g., Compounds 9 and 12. Without being bound by a particular theory, it appears that an electron withdrawing group such as fluorine or carbonyl substituted at the R³, R⁴, R⁶ or R⁷ position results in BAPTA chelating moieties that are particularly advantageous for chelating trivalent gallium ions that then also allows for the simultaneous interaction of the chelated gallium ion with an exposed phosphate group on the phosphorylated target molecules, resulting in a stable ternary complex.

The bridge substituents R⁹, R¹⁰, R¹¹ and R¹², are independently selected from the group consisting of hydrogen, lower alkyl, or adjacent substituents R⁹ and R¹⁰, taken in combination, constitute a 5-membered or 6-membered alicyclic or heterocyclic ring. R¹⁵, R⁶, R¹⁷ and R¹⁸ are independently H or lower alkyl; preferably R¹⁵, R¹⁶, R¹⁷ and R¹⁸ are all hydrogen. R¹³ and R¹⁴ are independently hydrogen, —CH₂OCOCH₃ or a salt.

It is understood that the chemical moieties of the present invention are attached to the BAPTA metal-chelating moiety by a linker at any of R¹-R¹² or alternatively the dye label comprises one of the aromatic rings of the metal-chelating moieties wherein no linker is present. Therefore, two adjacent substituents of R¹-R¹², when taken in combination with each other, and with the aromatic ring to which they are bound, comprise a fluorophore or chromophore label. However, a phosphate-binding compound could have more than one linker, such that a dye label is attached with no linker and four other linkers are present on the metal chelating compound to attach other labels or reactive groups. In one aspect of the invention, two adjacent ring substituents (R¹-R⁴ or R⁵-R⁸) taken in combination form the dye label that is a fused benzofuran or heteroaryl- or carboxyheteroaryl-substituted benzofuran fluorophore. Where the dye label is fused to the compound of the invention, it is preferably fused between R² and R³, or between R⁶ and R⁷.

Xanthene derivative dyes are particularly useful dyes of the present invention wherein, either or both of the benzene rings of the BAPTA or substituted BAPTA metal-binding compound is bonded to a xanthene ring through a single chemical bond, as in the common Ca²⁺ indicators fluo-3, fluo-4 and rhod-2 (U.S. Pat. No. 5,049,673, supra) or through the intermediacy of a phenyl or substituted phenyl spacer as in the Oregon Green® BAPTA indicators (U.S. Pat. No. 6,162,931, supra). The xanthene rings are typically bonded to the BAPTA at positions para to the nitrogen functions of the BAPTA. Particularly preferred are xanthene-containing BAPTA derivatives whose fluorophore is a rhodamine or a halogenated fluorescein. Particularly preferred are fluorescent BAPTA derivatives in which the 5-position of the BAPTA chelator is substituted by F, including rhod-5F and fluo-5F.

DTPA, as used herein, refers to diethylenetriamine pentaacetic acid chelating moieties and derivatives thereof, including any corresponding compounds disclosed in U.S. Pat. Nos. 4,978,763 and 4,647,447. DTPA metal-chelating moieties are represented by Formula II comprising

(CH₂CO₂R¹³)_{Z}N[(CH₂)_{S}N(CH₂CO₂R¹³)]_{T}(CH₂)_{S}N(CH₂CO₂R¹³)_{Z}

wherein a linker is attached to a methine carbon or nitrogen atom, Z is 1 or 2, S is 1 to 5, T is 0-4 and R¹³ is independently a hydrogen or a salt.

IDA, as used herein, refers to iminodiacetic acid compounds and derivatives thereof and is represented by Formula III comprising-(L)-N(CH₂CO₂R¹³)₂ wherein R¹³ is independently a hydrogen or a salt and provided that said linker is not a single covalent bond. The IDA metal-chelating moieties must be attached by a linker to a chemical moiety wherein the linker comprises at least one nonhydrogen atom. Without wishing to be bound by a theory, it appears that the linker increases the stability of the ternary complex and possibly tunes the affinity of the metal-chelating moiety for a metal ion of the present invention.

In addition to the above mentioned specific metal chelating moieties we have also found that phenanthroline based chelators also form ternary complex with metal ions and phosphate target molecules in a moderately acidic environment. Phenanthroline, as used herein, refers to 1,10-phenanthroline compounds and derivatives thereof and is represented by the structure

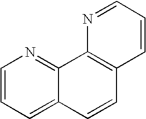

Any of the aromatic carbon atoms may be substituted with substituents well known to one skilled in the art, including those substituents disclosed in U.S. Pat. No. 6,316,267, supra. Alternatively, a linker can be attached to any of the aromatic carbon atoms to covalently attach a chemical moiety A to the phenanthroline moiety to form the phosphate-binding compounds of the present invention.

A suitable dye containing such a metal chelating moiety is commercially available as the Pro-Q Diamond stain (Molecular Probes). Suitable illustrative ProQ Diamond stains include the gel (MP33301) or microarray stain (MP33706).

Other detection systems that may be utilized include commercially available kits such as the PhosphoELISA (Biosource International) and fluorsence-based assays. Suitable fluorescence-based assay systems utilize reagents with novel metal binding amino acid residues exhibiting chelation-enhanced fluorescence (CHEF) upon binding to Mg²⁺ (see, for example, US 2005/0080242A2 and US 2005/0080243A1). Other systems are available to one of skill in the art and would be suitable in practicing the present invention.

### Substrates and Cofactors

In illustrative embodiments of the present invention, the substrate is or includes myelin basic protein (MBP), or a fragment or derivative thereof comprising at least 5. 10, 15, 20, 25, 50, 75, 100, 125, 150, or 175 contiguous amino acids of MBP, or one or more conservative substitutions thereof. Where the substrate is a fragment of MBP, the fragment typically includes at least 1, 2, 3, 4, 5, 6, 7, 8, 9, or 10 phosphorylation site(s) of MBP within the contiguous amino acids of MBP. The phosphorylation sites within an MBP fragment in certain embodiments, includes at least 1, 2, or 3 tyrosine residues. Furthermore, the MBP fragment can include different segments of MBP bound together, covalently or non-covalently.

A derivative of MBP is a polypeptide in which substitutions from the wild-teyp sequence are made on the basis of similarity in polarity, charge, solubility, hydrophobicity, hydrophilicity, and/or the amphipathic nature of the residues as long as the MBP derivative retains the ability to act as a substrate for a kinase that phosphorylates an identical residue of a wild type MBP. For example, substitutions of negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

A derivative of MBP is typically an MBP with conservative amino acid sequences. “Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having acidic side chains is glutamic acid and aspartic acid; a group of amino acids having amino-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

MBP refers to wild type mammalian MBP. This includes MBP from any mammal including, but not limited to, rat MBP, murine MBP, rabbit MBP, bovine MBP, and human MBP (SEQ ID NO:1).

In certain aspects, the MBP derivative shares at least 75%, 80%, 90%, 95%, 97%, 98%, or 99% identity with wild type MBP. The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

### Matrix: BLOSUM62

Open Gap: 11 and Extension Gap: 1 penalties
Gap x drop-off. 50

### Expect: 10

### Word Size: 3

### Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

In an illustrative embodiments, a substrate of an enzyme that catalyzes the phosphorylation of tyrosine residues in a protein or peptide is a protein or peptide (i.e. a tyrosine kinase substrate) with tyrosines. In another illustrative embodiment, a substrate of an enzyme that catalyzes the phosphorylation of serine and threonine residues in a protein or peptide (i.e. a serine/threonine kinase substrate) is a protein or peptide with a serine and/or threonine. A substrate for a dual specificity kinase has tyrosine and/or serine and/or threonine residues. Certain kinases require a conserved target motif in their substrate for phosphorylation. In certain embodiments, such a conserved target motif is present in the substrate. In a specific embodiment, a kinase substrate is, but is not limited to, myelin basic protein (MBP) or a derivative or fragment thereof that includes at least 10, 15, 20, or 25 amino acids of MBP including a residue that is phosphorylated.

The substrate can optionally include additional substrates in addition to MBP or a derivative or fragment thereof. For example, MBP and casein can be included. In another specific embodiment, a mixture of Myelin Basic Protein (MBP), histone and casein is used as substrate. In another specific embodiment, a mixture of Myelin Basic Protein (MBP), histone, casein and/or poly(Glu4Tyr) is used as substrate.

In illustrative embodiments, the MBP or derivative or fragment thereof, is not phosphorylated. For example, the MBP or derivative or fragment thereof can be a recombinant protein or peptide produced in a prokaryotic organism, such as E. coli. The MBP or derivative or fragment thereof can also be dephosphorylated as will be understood, before use in a method provided herein. In yet another specific embodiment, non-phosphorylated MBP is utilized as the substrate, for example as the sole substrate.

In still other embodiments, a “universal” substrate is provided. This substrate preferably comprises an amino acid sequence corresponding to MBP or a fragment or derivative thereof, as disclosed herein, joined to at least one amino acid sequence different from that of MBP, where both the MBP and the non-MBP amino acid sequence has the ability to serve as the substrate for a kinase. It is preferred that the non-MBP amino acid sequence is a substrate for one or more kinases that do not phosphorylate MBP. By joining multiple non-MBP amino acid sequences to the MBP sequence, a universal substrate is provided that may serve as a substrate for 2, 3, 4, 5, 10, 15, 20, 25, 50, 100, 150, 200, 250, 300, 400, 500, 750, 1000, or each kinase in a mammalian kinome, such as the human kinome. The non-MBP sequence(s) may be joined at the N-terminus of MBP, the C-terminus of MBP, or may be flanked by MBP sequence. It is preferred that the universal substrate is further joined to a purification tag such as GST, for the purpose of purification in a prokaryotic cell such as E. coli. In certain embodiments, multiple non-MBP sequences are adjacent to one another; in others, such sequences are separated by one or more linker(s) and/or MBP sequence(s). An exemplary universal substrate would be fused to a GST moiety at its N-terminus, directly adjacent to a full-length human MBP sequence, with one or more peptide sequences fused to the C-terminus of the MBP sequence. It is preferred that the universal substrate is not phosphorylated prior to use, in the assays of the present invention. As such, it may be useful to prepare non-phosphorylated myelin basic protein or the universal substrate synthetically or to express and purify the substrate from a prokaryotic host organism such as E. Coli. Exemplary non-MBP amino acid sequences useful in producing such a universal substrate include, for example, the kinase substrate peptides ALRRFSLGEK [SEQ ID NO 3], RGGLFSTTPGGTK [SEQ ID NO 4], VAPFSPGGRAK [SEQ ID NO 5], KLNRVFSVAC [SEQ ID NO 6], GDQDYLSLDK [SEQ ID NO 7], ARPRAFSVGK [SEQ ID NO 8], RRRQFSLRRKAK [SEQ ID NO 9], RPRTFSSLAEGK [SEQ ID NO 10], PRPFSVPPpSPDK [SEQ ID NO 11], KKKALSRQFSVAAK [SEQ ID NO 12], ESFSSSEEK [SEQ ID NO 13], VLAKSFGSPNRARKKk [SEQ ID NO 14], KKRPQRRYSNVL [SEQ ID NO 15], RRRLSFAEPG [SEQ ID NO 16], LVEPFTPSGEAPNQKK [SEQ ID NO 17, EVIEASFAEQEAK [SEQ ID NO 18], EEEIYGVIEK [SEQ ID NO 19], EAEAIYAAPGDK [SEQ ID NO 20], GVLTGYVARRK [SEQ ID NO 21], EEEEYIQIVK [SEQ ID NO 22], and AAEEIYAARRG [SEQ ID NO 23]. In certain embodiments, 1, 2, 3, 4, 5, 6, 7, 8, 9, 10, 11, 12, 13, 14, 15, 16, 17, 18, 19, 20, or all 21 peptides may be incorporated into the universal substrate.

Certain enzymes that use proteins or peptides as substrate require the presence of a particular amino acid or amino acid motif in their substrates for the enzymatic reaction to occur. Such sites in an amino acid sequence that are used by a particular enzymatic activity can be predicted using such databases as PROSITE. Such sequences may also be included within a universal substrate described herein, in addition to or in place of those sequences listed above.

### Cofactors

In certain embodiments of the invention, the enzymatic reaction being assayed requires a cofactor. Cofactors can be added to the reaction in the reaction mixture. Cofactors that can be used with the methods of the invention include, but are not limited to, 5,10-methenyltetrahydrofolate, Ammonia, Ascorbate, ATP, Bicarbonate, Bile salts, Biotin, Bis(molybdopterin guanine dinucleotide)molybdenum cofactor, Cadmium, Calcium, Cobalamin, Cobalt, Coenzyme F430, Coenzyme-A, Copper, Dipyrromethane, Dithiothreitol, Divalent cation, FAD, Flavin, Flavoprotein, FMN, Glutathione, Heme, Heme-thiolate, Iron, Iron(2+), Iron-molybdenum, Iron-sulfur, Lipoyl group, Magnesium, Manganese, Metal ions, Molybdenum, Molybdopterin, Monovalent cation, NAD, NAD(P)H, Nickel, Potassium, PQQ, Protoheme IX, Pyridoxal-phosphate, Pyruvate, Selenium, Siroheme, Sodium, Tetrahydropteridine, Thiamine pyrophosphate, Topaquinone, Tryptophan tryptophylquinone (TTQ), Tungsten, Vanadium, Zinc.

Properties of the Protein Chips to be Used with the Methods of the Invention

In various specific embodiments, the microarray of the invention is a positionally addressable array comprising a plurality of different kinases and a substrate immobilized on the surface of a solid support. In other embodiments, the microarray of the invention is a positionally addressable array comprising a plurality of different substrates and a kinase immobilized on the surface of a solid support. In certain embodiments, the kinases comprise a functional domain on a solid support. Each different kinase or substrate is at a different position on the solid support. In certain embodiments, the plurality of different kinases include at least 50%, 75%, 90%, or 95% of all expressed kinases in the genome of an organism, or at least 10, 100, 200, 250, 500, 1000, 2000, or 2500 kinases from the same organism. For example, such organism can be eukaryotic or prokaryotic, and is preferably a mammal, a human or non-human animal, primate, mouse, rat, cat, dog, horse, cow, chicken, fungus such as yeast, Drosophila, C. elegans, etc. Such biological activity of interest can be, but is not limited to, enzymatic activity such as kinase activity and other chemical group transferring enzymatic activity.

In certain embodiments, the plurality of different kinases or substrates is immobilized on the surface of the solid support at a density of about 1 to 10, 5 to 20, 10 to 50, 30 to 100, about 30, between 30 and 50, between 50 and 100, at least 100, between 100 and 1000, between 1000 and 10,000, between 10,000 and 100,000, between 100,000 and 1,000,000, between 1,000,000 and 10,000,000, between 10,000,000 and 25,000,000, at least 25,000,000, at least 10,000,000,000, or at least 10,000,000,000,000 different kinases or substrates, per cm.

In certain embodiments, the plurality of different kinases and a plurality of different substrates are immobilized on the surface of the solid support at a density of about 1 to 10, 5 to 20, 10 to 50, 30 to 100, about 30, between 30 and 50, between 50 and 100, at least 100, between 100 and 1000, between 1000 and 10,000, between 10,000 and 100,000, between 100,000 and 1,000,000, between 1,000,000 and 10,000,000, between 10,000,000 and 25,000,000, at least 25,000,000, at least 10,000,000,000, or at least 10,000,000,000,000 different kinases or substrates, respectively, per cm².

The protein chips to be used with the present invention are not limited in their physical dimensions and may have any dimensions that are convenient. For the sake of compatibility with current laboratory apparatus, protein chips the size of a standard microscope slide or smaller are preferred. In certain embodiments, protein chips are sized such that two chips fit on a microscope slide. Also preferred are protein chips sized to fit into the sample chamber of a mass spectrometer. Also preferred are microtiter plates.

In certain embodiments, a substrate and kinase are immobilized on the surface of a solid support within wells. In certain embodiments, a plurality of different kinases or different substrates is deposited or coated on the surface of the solid support such that each kinase or substrate of the microarray is in a different well. In other embodiments, a plurality of different kinases or different substrates is deposited onto the surface of the solid support such that each well harbors a plurality of different proteins or substrates. The performance of the kinase reaction on a solid support with wells has the advantage that different reaction solutions can be added at the same time onto one solid support (e.g., on one slide). Another advantage of wells over flat surfaces is increased signal-to-noise ratios. Wells allow the use of larger volumes of reaction solution in a denser configuration, and therefore greater signal is possible. Furthermore, wells decrease the rate of evaporation of the reaction solution from the chip as compared to flat surface arrays, thus allowing longer reaction times. Another advantage of wells over flat surfaces is that the use of wells permit association studies using a specific volume of reaction volume for each well on the chip, whereas the use of flat surfaces usually involves indiscriminate probe application across the whole substrate. The application of a defined volume of reaction buffer can be important if a reactant that is supplied in the reaction buffer is being depleted during the course of the reaction. In such a scenario, the application of a defined volume allows for more reproducible results.

In certain embodiments, if the microarrays to be used with the methods of the invention and the microarrays of the invention have wells, the wells in the protein chips may have any shape such as rectangular, square, or oval, with circular being preferred. The wells in the protein chips may have square or round bottoms, V-shaped bottoms, or U-shaped bottoms. Square bottoms are slightly preferred because the preferred reactive ion etch (RIE) process, which is anisotropic, provides square-bottomed wells. The shape of the well bottoms need not be uniform on a particular chip, but may vary as required by the particular assay being carried out on the chip.

The wells in the protein chips to be used with the methods of the present invention may have any width-to-depth ratio, with ratios of width-to-depth between about 10:1 and about 1:10 being preferred. The wells in the protein chips may have any volume, with wells having volumes of at least 1 pl, at least 10 pl, at least 100 pl, at least 1 nl, at least 10 nl, at least 100 nl, at least 11 μl, at least 10 μl, or at least 100 μl. The wells in the protein chips may have any volume, with wells having volumes of at most 1 pl, at most 10 pl, at most 100 pl, at most 1 nl, at most 10 nl, at most 100 nl, at most 1 μl, at most 10 μl, or at most 100 μl.

In certain embodiments, the wells are formed by placing a gasket with openings on the surface of the solid support such that the openings in the gasket form the wells. In certain, more specific embodiments, an array has at least 1, 2, 4, 6, 8, 10, 12, 14, 16, 18, 20, 24, 50 or at least 100 wells. In certain, more specific embodiments, an array has at most 1, 2, 4, 6, 8, 10, 12, 14, 16, 1, 20, 24, 50 or at least 100 wells.

In certain embodiments, the boundaries are formed by patterning a hydrophobic material with the pattern having openings to the surface of the solid support. Such openings in the pattern create hydrophilic regions surrounded by hydrophobic boundaries which are analogous to wells described above. In certain, more specific embodiments, an array has at least 1, 2, 4, 6, 8, 10, 12, 14, 16, 18, 20, 24, 50 or at least 100 hydrophilic regions. In certain, more specific embodiments, an array has at most 1, 2, 4, 6, 8, 10, 12, 14, 16, 18, 20, 24, 50 or at least 100 hydrophilic regions.

The protein chips of the invention can have a wide variety of density of wells/cm². The density of wells is between about 1 well/cm² and about 10,000,000,000,000 wells/cm². Densities of wells on protein chips cast from master molds of laser milled Lucite are generally between 1 well/cm² and 2,500 wells/cm². Appropriate milling tools produce wells as small as 100 μm in diameter and 100 μm apart. Protein chips cast from master mold etched by wet-chemical microlithographic techniques have densities of wells generally between 50 wells/cm² and 10,000,000,000 wells/cm². Wet-chemical etching can produce wells that are 10 μm deep and 10 μm apart, which in turn produces wells that are less than 10 μm in diameter. Protein chips cast from master mold etched by RIE microlithographic techniques have densities of wells generally between 100 wells/cm² and 25,000,000 wells/cm². RIE in combination with optical lithography can produce wells that are 500 nm in diameter and 500 nm apart. Use of electron beam lithography in combination with RIE can produce wells 50 nm in diameter and 50 nm apart. Wells of this size and with equivalent spacing produces protein chips with densities of wells 10,000,000,000,000 wells/cm². Preferably, RIE is used to produce wells of 20 μm in diameter and 20 μm apart. Wells of this size that are equivalently spaced will result in densities of 25,000,000 wells/cm².

In a specific embodiment, the microarray is prepared on a slide with 8 to 10 wells per slide, wherein the plurality of proteins is present in each well on the slide. In another embodiment, microarray is prepared on a slide with 8 to 10 wells per slide, wherein the plurality of substrates is present in each well on the slide.

In one embodiment, the array comprises a plurality of wells on the surface of a solid support wherein the density of wells is at least 1 well/cm², at least 10 wells/cm², 100 wells/cm². In another embodiment, said density of wells is between 100 and 1000 wells/cm². In another embodiment, said density of wells is between 1000 and 10,000 wells/cm². In another embodiment, said density of wells is between 10,000 and 100,000 wells/cm². In yet another embodiment, said density of wells is between 100,000 and 1,000,000 wells/cm². In yet another embodiment, said density of wells is between 1,000,000 and 10,000,000 wells/cm². In yet another embodiment, said density of wells is between 10,000,000 and 25,000,000 wells/cm². In yet another embodiment, said density of wells is at least 25,000,000 wells/cm². In yet another embodiment, said density of wells is at least 10,000,000,000 wells/cm². In yet another embodiment, said density of wells is at least 10,000,000,000,000 wells/cm².

The placement of a kinase(s) or a substrate(s) can be accomplished by using any dispensing means, such as bubble jet or ink jet printer heads. A micropipette dispenser can also be used. The placement of proteins or probes can either be conducted manually or the process can be automated through the use of a computer connected to a machine.

The present invention contemplates a variety of solid supports cast from a microfabricated mold, some of which are disclosed, for example, in international patent application publication WO 01/83827, published Nov. 8, 2001, which is incorporated herein by reference in its entirety.

### Methods for Making and Purifying Proteins

Any method known to the skilled artisan can be used to make and to purify the kinases to be used with the methods of the invention and for the preparation of the microarrays of the invention. In certain embodiments, the substrate is also a proteinaceous molecule, such as a protein, a polypeptide or a peptide and can be prepared and purified as described in this section.

Proteins to be used with the methods of the invention and for the preparation of the microarrays of the invention can be fusion proteins, in which a defined domain is attached to one of a variety of natural proteins, or can be intact non-fusion proteins. In certain embodiments, if the substrate is a protein or a peptide, a substrate to be used with the methods of the invention and for the preparation of the microarrays of the invention can be fusion protein, in which a defined domain is attached to the substrate, or can be intact non-fusion substrate.

The present invention also relates to methods for making and isolating viral, prokaryotic or eukaryotic proteins in a readily scalable format, amenable to high-throughput analysis. Preferred methods include synthesizing and purifying proteins in an array format compatible with automation technologies. Accordingly, in one embodiment, the invention provides a method for making and isolating eukaryotic proteins comprising the steps of growing a eukaryotic cell transformed with a vector having a heterologous sequence operatively linked to a regulatory sequence, contacting the regulatory sequence with an inducer that enhances expression of a protein encoded by the heterologous sequence, lysing the cell, contacting the protein with a binding agent such that a complex between the protein and binding agent is formed, isolating the complex from cellular debris, and isolating the protein from the complex, wherein each step is conducted, e.g., in a 96-well format.

In certain embodiments, the plurality of proteins comprises at least one protein with a first tag and a second tag. In yet another embodiment, the plurality of substrates comprises at least one substrate with a first tag and a second tag.

In one embodiment, each step in the synthesis and purification procedures is conducted in an array amenable to rapid automation. Such arrays can comprise a plurality of wells on the surface of a solid support wherein the density of wells is at least 10, 20, 30, 40, 50, 100, 1000, 10,000, 100,000, or 1,000,000 wells/cm², for example. Alternatively, such arrays comprise a plurality of sites on the surface of a solid support, wherein the density of sites is at least 10, 20, 30, 40, 50, 100, 1000, 10,000, 100,000, or 1,000,000 sites/cm², for example.

In a particular embodiment, proteins and/or substrates are made and purified in a 96-array format (i.e., each site on the solid support where processing occurs is one of 96 sites), e.g., in a 96-well microtiter plate. In a preferred embodiment, the surface of the microtiter plate that is used for the production of the proteins and/or substrates does not bind proteins (e.g., a non-protein-binding microtiter plate).

In certain embodiments, proteins and/or substrates are synthesized by in vitro translation according to methods commonly known in the art.

Any expression construct having an inducible promoter to drive protein synthesis and/or the synthesis of a substrate (if the substrate(s) is a protein or peptide) can be used in accordance with the methods of the invention. Preferably, the expression construct is tailored to the cell type to be used for transformation. Compatibility between expression constructs and host cells are known in the art, and use of variants thereof are also encompassed by the invention.

Any host cell that can be grown in culture can be used to synthesize the proteins and/or substrates of interest. Preferably, host cells are used that can overproduce a protein and/or a substrate of interest, resulting in proper synthesis, folding, and posttranslational modification of the protein. Preferably, such protein processing forms epitopes, active sites, binding sites, etc. useful for the activity of an enzyme or the suitability as a substrate. Posttranslational modification is relevant if the enzyme's activity is affected by posttranslational modification of the enzyme. Posttranslational modification is also relevant if the substrates ability to serve as a substrate for the enzymatic reaction of interest is affected by the posttranslational modification of the substrate. In a specific embodiment, phosphorylation of a protein is required for the enzymatic activity of the protein. In such a case the protein should be expressed in a system that promotes the phosphorylation of the protein at the appropriate site. In a specific embodiment, phosphorylation or glycosylation of a substrate is required for the substrate to modified by the enzymatic reaction of interest. In such a case the substrate should be synthesized in a system that promotes the phosphorylation or glycosylation of the substrate at the appropriate site.

Accordingly, a eukaryotic cell (e.g., yeast, human cells) is preferably used to synthesize eukaryotic proteins or substrates of eukaryotic enzymes. Further, a eukaryotic cell amenable to stable transformation, and having selectable markers for identification and isolation of cells containing transformants of interest, is preferred. Alternatively, a eukaryotic host cell deficient in a gene product is transformed with an expression construct complementing the deficiency. Cells useful for expression of engineered viral, prokaryotic or eukaryotic proteins are known in the art, and variants of such cells can be appreciated by one of ordinary skill in the art.

For example, the InsectSelect system from Invitrogen (Carlsbad, Calif., catalog no. K800-01), a non-lytic, single-vector insect expression system that simplifies expression of high-quality proteins and eliminates the need to generate and amplify virus stocks, can be used. A preferred vector in this system is pIB/V5-His TOPO TA vector (catalog no. K890-20). Polymerase chain reaction (“PCR”) products can be cloned directly into this vector, using the protocols described by the manufacturer, and the proteins can be expressed with N-terminal histidine tags useful for purifying the expressed protein.

Another eukaryotic expression system in insect cells, the BAC-TO-BAC™ system (LIFETECH™, Rockville, Md.), can also be used. Rather than using homologous recombination, the BAC-TO-BAC™ system generates recombinant baculovirus by relying on site-specific transposition in E. coli. Gene expression is driven by the highly active polyhedrin promoter, and therefore can represent up to 25% of the cellular protein in infected insect cells.

In a particular embodiment, yeast cultures are used to synthesize eukaryotic fusion proteins. Fresh cultures are preferably used for efficient induction of protein synthesis, especially when conducted in small volumes of media. Also, care is preferably taken to prevent overgrowth of the yeast cultures. In addition, yeast cultures of about 3 ml or less are preferable to yield sufficient protein for purification. To improve aeration of the cultures, the total volume can be divided into several smaller volumes (e.g., four 0.75 ml cultures can be prepared to produce a total volume of 3 ml).

Cells are then contacted with an inducer, and harvested. The nature of the inducer depends on the expression system used. The nature of the inducer particularly depends on the promoter used. In certain embodiments, the expression system used for the preparation of the proteins and/or substrates is an inducible expression system. Any inducible expression system known to the skilled artisan can be used with the methods of the invention and for the preparation of the microarrays of the invention. Examples of inducers include, but are not limited to, galactose, enhancer-binding proteins, and other transcription factors. In one embodiment, galactose is contacted with a regulatory sequence comprising a galactose-inducible GAL1 promoter.

Induced cells are washed with cold (i.e., 4° C. to about 15° C.) water to stop further growth of the cells, and then washed with cold (i.e., 4° C. to about 15° C.) lysis buffer to remove the culture medium and to precondition the induced cells for protein purification, respectively. Before protein purification, the induced cells can be stored frozen to protect the proteins from degradation. In a specific embodiment, the induced cells are stored in a semi-dried state at −80° C. to prevent or inhibit protein degradation.

Cells can be transferred from one array to another using any suitable mechanical device. For example, arrays containing growth media can be inoculated with the cells of interest using an automatic handling system (e.g., automatic pipette). In a particular embodiment, 96-well arrays containing a growth medium comprising agar can be inoculated with yeast cells using a 96-pronger. Similarly, transfer of liquids (e.g., reagents) from one array to another can be accomplished using an automated liquid-handling device (e.g., Q-FILL™, Genetix, UK).

Although proteins can be harvested from cells at any point in the cell cycle, cells are preferably isolated during logarithmic phase when protein synthesis is enhanced. For example, yeast cells can be harvested between OD₆₀₀=0.3 and OD₆₀₀=1.5, preferably between OD₆₀₀=0.5 and OD₆₀₀=1 0.5. In a particular embodiment, proteins are harvested from the cells at a point after mid-log phase. Harvested cells can be stored frozen for future manipulation.

The harvested cells can be lysed by a variety of methods known in the art, including mechanical force, enzymatic digestion, and chemical treatment. The method of lysis should be suited to the type of host cell. For example, a lysis buffer containing fresh protease inhibitors is added to yeast cells, along with an agent that disrupts the cell wall (e.g., sand, glass beads, zirconia beads), after which the mixture is shaken violently using a shaker (e.g., vortexer, paint shaker).

In a specific embodiment, zirconia beads are contacted with the yeast cells, and the cells lysed by mechanical disruption by vortexing. In a further embodiment, lysing of the yeast cells in a high-density array format is accomplished using a paint shaker. The paint shaker has a platform that can firmly hold at least eighteen 96-well boxes in three layers, thereby allowing for high-throughput processing of the cultures. Further the paint shaker violently agitates the cultures, even before they are completely thawed, resulting in efficient disruption of the cells while minimizing protein degradation. In fact, as determined by microscopic observation, greater than 90% of the yeast cells can be lysed in under two minutes of shaking.

The resulting cellular debris can be separated from the protein and/or substrate of interest by centrifugation. Additionally, to increase purity of the protein sample in a high-throughput fashion, the protein-enriched supernatant can be filtered, preferably using a filter on a non-protein-binding solid support. To separate the soluble fraction, which contains the proteins of interest, from the insoluble fraction, use of a filter plate is highly preferred to reduce or avoid protein degradation. Further, these steps preferably are repeated on the fraction containing the cellular debris to increase the yield of protein.

Proteins and/or substrates can then be purified from the protein-enriched supernatant using a variety of affinity purification methods known in the art. Affinity tags useful for affinity purification of fusion proteins by contacting the fusion protein preparation with the binding partner to the affinity tag, include, but are not limited to, calmodulin, trypsin/anhydrotrypsin, glutathione, immunoglobulin domains, maltose, nickel, or biotin and its derivatives, which bind to calmodulin-binding protein, bovine pancreatic trypsin inhibitor, glutathione-S-transferase (“GST tag”), antigen or Protein A, maltose binding protein, poly-histidine (“His tag”), and avidin/streptavidin, respectively. Other affinity tags can be, for example, myc or FLAG. Fusion proteins can be affinity purified using an appropriate binding compound (i.e., binding partner such as a glutathione bead), and isolated by, for example, capturing the complex containing bound proteins on a non-protein-binding filter. Placing one affinity tag on one end of the protein (e.g., the carboxy-terminal end), and a second affinity tag on the other end of the protein (e.g., the amino-terminal end) can aid in purifying full-length proteins.

In certain embodiments, a protein and/or a substrate is expressed as a fusion protein with a chitin binding domain. In other embodiments, a protein and/or a substrate is expressed as a fusion protein with a chitin binding domain and an intein. In a more specific embodiment, the proteins and/or substrates are expressed using the IMPACT™-CN system from New England Biolabs Inc.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration.

For purified proteins and/or substrates that will eventually be deposited or coated onto the surface of the solid support, such as, but not limited to, a microscope slide, the glutathione beads are separated from the purified proteins and/or substrates. Preferably, all of the glutathione beads are removed to avoid blocking of the microarrays pins used to spot the purified proteins onto a solid support. In a preferred embodiment, the glutathione beads are separated from the purified proteins using a filter plate, preferably comprising a non-protein-binding solid support. Filtration of the eluate containing the purified proteins should result in greater than 90% recovery of the proteins.

The elution buffer preferably comprises a liquid of high viscosity such as, for example, 15% to 50% glycerol, preferably about 25% glycerol. The glycerol solution stabilizes the proteins and/or substrates in solution, and prevents dehydration of the protein solution during the printing step using a microarrayer.

Purified proteins and/or substrates are preferably stored in a medium that stabilizes the proteins and prevents dessication of the sample. For example, purified proteins can be stored in a liquid of high viscosity such as, for example, 15% to 50% glycerol, preferably in about 25% glycerol. It is preferred to aliquot samples containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

In certain embodiments, an enzyme to be used with the invention is composed of two or more proteins in a complex. In such a case, any method known to the skilled artisan can be used to provide the complex for use with the methods of the invention. In a specific embodiment, the proteins of the complex are co-expressed and the proteins are purified as a complex. In other embodiments, the proteins of the complex are expressed as a fusion protein that comprises all proteins of the complex. The fusion protein may or may not comprise linker peptides between the individual proteins of the complex. In other embodiments, the proteins of the complex are expressed, purified and subsequently incubated under conditions that allow formation of the complex. In certain embodiments, the proteins of the complex are assembled on the surface of the solid support before they become immobilized. In even other embodiments, the individual proteins of an enzymatic complex of interest are deposited on top of each other on the surface of the solid support. Without being bound by theory, once the proteins of the complex are immobilized on the surface the close physical proximity of the proteins of the complex to each other allows for the enzymatic reaction to take place even though the complex is not assembled.

The protein and/or substrate can be purified prior to placement on the protein chip or can be purified during placement on the chip via the use of reagents that bind to particular proteins, which have been previously placed on the protein chip. Partially purified protein-containing cellular material or cells can be obtained by standard techniques (e.g., affinity or column chromatography) or by isolating centrifugation samples (e.g., P1 or P2 fractions).

### Tagged Proteins

In certain embodiments, the proteins and/or substrates to be used with the methods of the invention or for the preparation of the microarrays of the invention comprise a first tag and a second tag. The advantages of using double-tagged proteins include the ability to obtain highly purified proteins, as well as providing a streamlined manner of purifying proteins from cellular debris and attaching the proteins to a solid support. In a particular embodiment, the first tag is a glutathione-S-transferase tag (“GST tag”) and the second tag is a poly-histidine tag (“His tag”). In a specific embodiment, the poly-histidine tag consists of six histidines (Hisx6). In other embodiments, the poly-histidine tag consists of 4, 5, 7, 8, 9, 10, 11, or 12 histidines. In a further embodiment, the GST tag and the His tag are attached to the amino-terminal end of the protein or the substrate. Alternatively, the GST tag and the His tag are attached to the carboxy-terminal end of the protein or substrate.

In a preferred embodiment, a protein and/or a substrate is expressed using the IMPACT™-CN system from New England Biolabs Inc.

In yet another embodiment, the GST tag is attached to the amino-terminal end of the protein or substrate. In a further embodiment, the His tag is attached to the carboxy-terminal end of the protein or substrate. In yet another embodiment, the His tag is attached to the amino-terminal end of the protein or substrate. In a further embodiment, the GST tag is attached to the carboxy-terminal end of the protein or substrate.

In yet another embodiment, the protein or substrate comprises a GST tag and a His tag, and neither the GST tag nor the His tag is located at the amino-terminal or carboxy-terminal end of the protein. In a specific embodiment, the GST tag and His tag are located within the coding region of the protein or substrate of interest; preferably in a region of the protein not affecting the enzymatic activity of interest and preferably in a region of the substrate not affecting the suitability of the substrate to be modified by the enzymatic reaction of interest.

In one embodiment, the first tag is used to purify a fusion protein. In another embodiment, the second tag is used to attach a fusion protein to a solid support. In a specific further embodiment, the first tag is a GST tag and the second tag is a His tag.

A binding agent that can be used to purify a protein or a substrate can be, but is not limited to, a glutathione bead, a nickel-coated solid support, and an antibody. In one embodiment, the complex comprises a fusion protein having a GST tag bound to a glutathione bead. In another embodiment, the complex comprises a fusion protein having a His tag bound to a nickel-coated solid support. In yet another embodiment, the complex comprises the protein of interest bound to an antibody and, optionally, a secondary antibody.

### Screening Assays

The methods of the invention and the protein microarrays of the invention can be used to identify molecules that modify kinase activity or a kinase substrate-specificity. In particular, the methods of the invention and the protein microarrays of the invention can be used to identify a molecule with a particular profile of activity, i.e., the molecule modifies certain kinases and does not affect the activity of other kinases. Such an assay is particularly useful to identify compounds that are modulators of a desired specificity, wherein the compound with the highest specificity modifies the activity of only one specific kinase and a compound with a lower specificity modifies the activity of a subclass of kinases. Modulators of an enzymatic activity can be activators of the kinase activity, inhibitors of the kinase activity or modulators of the kinase substrate specificity. An inhibitor of an enzymatic reaction can inhibit the kinase reversably, irreversably, competitively, or non-competitively.

In certain embodiments, a screening assay of the invention is performed by conducting the kinase assay on a microarray as described herein, wherein the reaction is performed in the presence and the absence of a molecule that is to be tested for its effect on the kinase reaction. The effect of the test molecule on the kinase reaction can be determined by comparing the activity in the presence of the test molecule with the activity in the absence of the test compound. In certain embodiments, if the assay is performed in wells, several molecules can be tested simultaneously on the same microarray. In certain embodiments, if the assay is performed in wells, different concentrations of a molecule can be tested simultaneously on the same microarray.

In certain embodiments, a molecule is tested for its effect on the activity of a kinase reaction, wherein a plurality of different kinases and a substrate are immobilized to the surface of the solid support. In a specific embodiment, the substrate may be a known substrate of at least one of the kinases. This is the preferred embodiment, if the molecule is tested for an effect on kinase activity. If substrate specificity of a kinase of interest is to be tested, the preferred embodiment is to perform the assay on a microarray wherein a plurality of different substrates and the kinase of interest are immobilized on the surface of a solid support.

In other embodiments, the methods of the invention and the microarrays of the invention can be used to identify a substrate that is utilized by a kinase of interest, or a kinase subclass of interest.

In certain embodiments, the methods of the invention are used to determine a profile of kinase activities of a cell in a particular state of development or proliferation or of a cell of a particular cell type. In a specific embodiment, the methods of the invention are used to determine a profile of kinase activities of a cell that is pre-neoplastic, neoplastic or cancerous in comparison to a non-neoplastic or non-cancerous, respectively, cell. In a specific embodiment, a cell extract of a cell type of interest is immobilized on the surface of a solid support and a plurality of different kinase substrates is also immobilized on the surface. In a more specific embodiment, the cell extract is size fractionated and the different fractions are used with the methods of the invention to enrich for the kinases of interest in the cell extract. In an even more specific embodiment, at least one kinase is isolated from a cell of interest and tested for its activity using the methods of the invention.

In certain embodiments, kinetic properties of a known inhibitor of a certain kinase are assessed using the methods of the invention. In certain, more specific embodiments, at least 1, 2, 3, 4, 5, 6, 7, 8, 9, 10, 12, 14, 16, 18, 20, 30, 40, 50 or at least 100 copies of the plurality of different kinases are immobilized on the surface of a solid support at different positions of the microarray. The different kinases of at least 1 copy of the plurality of different kinases on the microarray are in proximity with a substrate sufficient for the occurrence of an enzymatic reaction between the kinase of the plurality of different kinases and the substrate. The different copies of the plurality of different kinases can then incubated with different reaction mixtures. The different reaction mixtures can each contain a different test molecule that is to be tested for its effect on the kinase reaction being assayed. In other embodiments, the different reaction mixtures can each contain a different concentration of a test molecule or known inhibitor or activator of the kinase reaction. In certain embodiments, the different copies of the plurality of different kinases are in different wells on the solid support.

In certain, more specific embodiments, a at least 1, 2, 3, 4, 5, 6, 7, 8, 9, 10, 12, 14, 16, 18, 20, 30, 40, 50 or at least 100 copies of a plurality of different substrates are immobilized on the surface of a solid support at different positions of the microarray. The different substrates of at least 1 copy of the plurality of different substrates on the microarray are in proximity with a kinase sufficient for the occurrence of an enzymatic reaction between the substrates of the plurality of different substrate and the kinase. The different copies of the plurality of different substrates can then incubated with different reaction mixtures. The different reaction mixtures can each contain a different test molecule that is to be tested for its effect on the kinase reaction being assayed. In other embodiments, the different reaction mixtures can each contain a different concentration of a test molecule or known inhibitor or activator of the enzymatic reaction. In certain embodiments, the different copies of the plurality of different substrates are in different wells on the solid support.

In certain embodiments, the IC₅₀ of an inhibitor of a kinase reaction can be determined. As described above, different concentrations of the inhibitor can be tested for their effects on a kinase reaction. Based on the different effects of different concentrations of the inhibitor on the kinase reaction, the IC₅₀ can be determined. In a specific embodiment, a dose-response curve is established based on the different effects of different concentrations of the inhibitor on the kinase reaction, wherein the IC₅₀ is the concentration of the inhibitor where the kinase activity is 50% of the activity in the absence of inhibitor.

In certain illustrative examples, provided herein is a method for identifying a test molecule that modulates an kinase reaction, including:

(a) incubating at least one kinase, at least one substrate, and at least one test molecule under conditions conducive to the occurrence of an enzymatic reaction between the kinase and the substrate (i.e. a reaction involving the substrate that is catalyzed by the kinase), wherein (i) the kinase and the substrate are immobilized on the surface of a solid support; (ii) the kinase and the substrate are in proximity sufficient for the occurrence of said enzymatic reaction; and (iii) the kinase and the substrate are not identical; and

(b) determining whether the kinase reaction is modulated by the test molecule. Typically, the kinase and the substrate are immobilized before the incubation step.

In one illustrative example, a plurality of substrates are coated onto the surface of the solid support and a plurality of kinases are deposited onto the surface of the solid support before the incubation step, and the method identifies test molecules that modulate phosphorylation of the substrate by the kinase during the incubation step.

### Libraries of Molecules

Any molecule known to the skilled artisan can be used with the methods of the invention to test the molecule's effect on the kinase reaction being assayed. In other embodiments, any molecule can be used as a candidate substrate with the methods of the invention. For example, a test molecule can be a polypeptide, carbohydrate, lipid, amino acid, nucleic acid, fatty acid, steroid, or a small organic compound. In addition, a test molecule can be lipophilic, hydrophilic, plasma membrane permeable, or plasma membrane impermeable. The molecule can be of natural origin or synthetic origin The test molecule can be a small molecule, such as a synthetic compound.

In certain embodiments, a library of different molecules is used with the methods of the invention, or an individual molecule is used with the methods of the invention, from a library of different molecules or of the same chemical class as the molecules discussed in this section, as non-limiting examples. One or more members of a library, including, for example, each member of a library, can be used as a test molecule to test its effect on the enzymatic reaction or as a substrate to test its suitability as a substrate for the reaction being assayed.

In certain embodiments, the members of the library are tested individually. In other embodiments, the members of a library are tested initially in pools. The size of a pool can be at least 2, 10, 50, 100, 500, 1000, 5,000, or at least 10,000 different molecules. Once a positive pool is identified, fractions of the pool can be tested or the individual members of the pool of molecules are tested.

Libraries can contain a variety of types of molecules. Examples of libraries that can be screened in accordance with the methods of the invention include, but are not limited to, peptoids; random biooligomers; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; carbohydrate libraries; and small molecule libraries (preferably, small organic molecule libraries). In some embodiments, the molecules in the libraries screened are nucleic acid or peptide molecules. In a non-limiting example, peptide molecules can exist in a phage display library. In other embodiments, the types of compounds include, but are not limited to, peptide analogs including peptides comprising non-naturally occurring amino acids, e.g., D-amino acids, phosphorous analogs of amino acids, such as α-amino phosphoric acids and α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used in the assays of the invention.

In certain embodiments, combinatorial libraries of small organic molecules including, but not limited to, benzodiazepines, isoprenoids, thiazolidinones, metathiazanones, pyrrolidines, morpholino compounds, and benzodiazepines. In another embodiment, the combinatorial libraries comprise peptoids; random bio-oligomers; benzodiazepines; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; or carbohydrate libraries can be used with the methods of the invention. Combinatorial libraries are themselves commercially available (see, e.g., ComGenex, Princeton, N.J.; Asinex, Moscow, Ru, Tripos, Inc., St. Louis, Mo.; ChemStar, Ltd, Moscow, Russia; 3D Pharmaceuticals, Exton, Pa.; Martek Biosciences, Columbia, Md.; etc.).

In a preferred embodiment, the library is preselected so that molecules of the library are of the general type of molecules that are being used in the enzymatic reaction of interest.

The combinatorial molecule library for use in accordance with the methods of the present invention may be synthesized. There is a great interest in synthetic methods directed toward the creation of large collections of small organic compounds, or libraries, which could be screened for pharmacological, biological or other activity. The synthetic methods applied to create vast combinatorial libraries are performed in solution or in the solid phase, i.e., on a solid support. Solid-phase synthesis makes it easier to conduct multi-step reactions and to drive reactions to completion with high yields because excess reagents can be easily added and washed away after each reaction step. Solid-phase combinatorial synthesis also tends to improve isolation, purification and screening. However, the more traditional solution phase chemistry supports a wider variety of organic reactions than solid-phase chemistry.

Combinatorial molecule libraries to be used in accordance with the methods of the present invention may be synthesized using the apparatus described in U.S. Pat. No. 6,190,619 to Kilcoin et al., which is hereby incorporated by reference in its entirety. U.S. Pat. No. 6,190,619 discloses a synthesis apparatus capable of holding a plurality of reaction vessels for parallel synthesis of multiple discrete compounds or for combinatorial libraries of compounds.

In one embodiment, the combinatorial molecule library can be synthesized in solution. The method disclosed in U.S. Pat. No. 6,194,612 to Boger et al., which is hereby incorporated by reference in its entirety, features compounds useful as templates for solution phase synthesis of combinatorial libraries. The template is designed to permit reaction products to be easily purified from unreacted reactants using liquid/liquid or solid/liquid extractions. The compounds produced by combinatorial synthesis using the template will preferably be small organic molecules. Some compounds in the library may mimic the effects of non-peptides or peptides. In contrast to solid phase synthesize of combinatorial compound libraries, liquid phase synthesis does not require the use of specialized protocols for monitoring the individual steps of a multistep solid phase synthesis (Egner et al., 1995, J. Org. Chem. 60:2652; Anderson et al., 1995, J. Org. Chem. 60:2650; Fitch et al., 1994, J. Org. Chem. 59:7955; Look et al., 1994, J. Org. Chem. 49:7588; Metzger et al., 1993, Angew. Chem., Int. Ed. Engl. 32:894; Youngquist et al., 1994, Rapid Commun. Mass Spect. 8:77; Chu et al., 1995, J. Am. Chem. Soc. 117:5419; Brummel et al., 1994, Science 264:399; and Stevanovic et al., 1993, Bioorg. Med. Chem. Lett. 3:431).

Combinatorial molecule libraries useful for the methods of the present invention can be synthesized on solid supports. In one embodiment, a split synthesis method, a protocol of separating and mixing solid supports during the synthesis, is used to synthesize a library of compounds on solid supports (see e.g., Lam et al., 1997, Chem. Rev. 97:41-448; Ohlmeyer et al., 1993, Proc. Natl. Acad. Sci. USA 90:10922-10926 and references cited therein). Each solid support in the final library has substantially one type of compound attached to its surface. Other methods for synthesizing combinatorial libraries on solid supports, wherein one product is attached to each support, will be known to those of skill in the art (see, e.g., Nefzi et al., 1997, Chem. Rev. 97:449-472).

In certain embodiments of the invention, the compound is a small molecule (less than 10 kDa), e.g., a non-peptide small molecule.

The examples set forth below illustrate but do not limit the invention.

### EXAMPLES

### Example I

### Kinase Activity Assay on Microarray

### Materials & Reagents

| Materials/Equipment/ | | |
|---|---|---|
| Reagents | Vendor | Part Number |
| Disposables/Reagents | | |
| Gamma- AT³³P (10 μCi/μl, | Perkin Elmer | NEG602H250UC |
| 250 μCi) | | |
| Histone, Calf Thymus | Calbiochem | 38205 |
| Casein | Sigma | C-4032 |
| Myelin Basic Protein | Sigma | M-1891 |
| Poly-glutamic acid-tyrosine | Sigma | P-2075 |
| PBS Tablets | American | AB11108 |
| | Bioanalytical | |
| Tween-20 | American | AB02038 |
| | Bioanalytical | |
| 60 × 24 mm Hybridization | Schleicher & | 10 484 907 |
| Cover slips | Schuell | |
| Equipment | | |
| Cyclone Phospho-imager | Perkin Elmer | B431220 |
| 8 × 10 Autoradiography | Fisher | FB-XC-810 |
| Cassettes | | |
| Phosphor Storage Screens | Perkin Elmer | 7001723 |
| (MS) | | |
| Lab Rotator | Lab-Line | 1314 |
| | Instruments | |
| Eppendorf Centrifuge | Fisher Scientific | 05-400-60 |
| (5810) | | |

### Reagent/Stock Preparation

a) Kinase Substrate Stocks
Dissolve protein substrates in 20 mM Tris to a final concentration of 10 mg/mL.

b) 1 L of 1×PBS
Dissolve 5 PBS tablets in 1 L dH₂O. Mix thoroughly.

c) 1 L PBST
Dissolve 5 PBS tablets in 1 L dH₂O. Add 1 mL Tween-20. Mix thoroughly.

d) Kinase Assay Dilution Buffer
20 mM MOPS, pH 7.2, 25 mM b-glycerol phosphate, 5 mM EGTA, 1 mM sodium orthovanadate
   Assay Solution (1 ml nominal—total=˜1.1 ml)

| | |
|---|---|
| In 1 ml of Kinase Assay Dilution Buffer, add | (~final concentration) |
| 1 μl of 1 M DTT | (1 mM) |
| 1 μl of 30% BSA | (3 mg/ml) |
| 1 μl of 1 M MnCl₂ | (1 mM) |
| 1 μl of 1 M CaCl₂ | (1 mM) |
| 25 μl of 1 M MgCl₂ | (25 mM) |

### Methods

Step 1: Coating of Slides with Kinase Substrates

To coat slides with kinase substrate, the substrates are diluted to 10 ng/μL in 1×PBS and 180-200 μL of substrate solution are pipetted onto one slide, e.g., a glass slide, aldehyde treated slides (TeleChem International, Inc.), nitrocellulose-coated slides (Schleicher & Schuell), slides with an amino-silane surface (Corning). A second slide is then placed on top of the first slide so that the sides to be deposited with kinases face each other. Care should be taken that the liquid covers the entire slide and that there are no air bubbles. The slides are placed in a 50 mL conical tube, making sure they are laying flat and incubated at 4° C. for one hour to several days.

Alternatively, substrates may be deposited on the slides using a microarrayer, wherein the samples are kept at 4° C. The substrates should be diluted in the proper printing buffer. The spot size should be 150-200 μm, and the spacing should be between 0.5 and 1 mm. After printing, incubate at 4° C. for one hour to several days.

### Step 2: Washing and Blocking of Coated Slides

The substrate-coated or substrate-deposited slides obtained in step 1 are removed from the conical tubes and placed in a slide staining dish. Subsequently, approximately 100 mL of PBST are added to the dish. The slides are then washed for one hour at 4° C. with shaking. The PBST is then discarded and the slides are gently rinsed with dH2O using a squirt bottle. After rinsing, the slides are placed into a slide boxes and centrifuged at 4000 rpm for one minute. The slides are then stored at 4° C. until printing with kinase.

### Step 3: Printing of Kinases on Substrate-Coated Slides

Kinases are diluted in the proper printing buffer. The concentration should be between 1 and 10 ng/μL. The kinases are deposited on the substrate-coated slides obtained in step 1 and 2 using a microarrayer. The spot size should be 150-200 μm, and the spacing should be between 0.5 and 1 mm. If the substrate is deposited on the slides, the spacing of the kinase array should match that of the substrate array (i.e., the kinases should be deposited on top of the substrate). The slides can be stored at 4° C. until the kinase activity assay is performed.

### Step 4: Assay of Kinase Activity on Microarray

1 mL of kinase assay buffer for every 12 glass slides to be probed is prepared. 6 μL of gamma-AT³³P (10 μCi/μL) are added to the assay buffer. The slides are placed in 50 mL conical tubes, laying flat, proteins facing up. 70 μL to 150 μL of the kinase assay buffer with gamma-AT³³P are added onto each slide. Using tweezers, the slide is covered with a hybridization slip, making sure that the solution completely covers the microarray.

The conical tube is then closed and placed in a 30° C. incubator. Care should be taken that the slide is laying flat. The reaction is then incubated for 90 minutes. Subsequently, the tubes are removed from the incubator. Approximately 40 mL of dH₂O are added to each tube and, using the tweezers, the hybridization slip is removed, the tube is closed and inverted several times for 1-2 minutes to rinse the slide inside the conical tube. The wash solution is then discarded. Approximately 40 mL of dH₂O are added again to each tube, the tubes are closed and inverted several times for 1-2 minutes, the wash solution is discarded. The slides are then removed from the tubes and place in a slide box and centrifuged at 4000 rpm for 1-2 minutes.

A phosphor screen (suitable for ³³P) is re-activated for each membrane by exposing it to light for at least 30 minutes. A piece of filter paper is placed in an autoradiography cassette and the dried slides are placed on the filter paper, facing up. The slides are covered with a piece of clear plastic film (such as SaranWrap). The phosphor screen is placed on top of the SaranWrap, facing the slides. The cassette is then closed and locked and exposed for a few hours to a couple of days, depending on the activity. In a dark room (or a room with dim light), the cassette is opened and the phosphor screen is removed. The phosphor screen is then mounted on the Cyclone rotor and scanned at 600 dpi.

It has been determined that the substrate is required for the kinase reaction to take place. Thus, the signal obtained in this experiment is due to specific phosphorylation of the substrate and not due to autophosphorylation or binding of the labeled ATP to some of the enzymes.

It has also been determined that treatment of the slide with aldehyde improves the signal-to-noise ratio. The experiments were conducted essentially using the method described above but with different types of slides. The aldehyde-treated slides were obtained from TeleChem International, Inc. The slide shown as FAST is a nitrocellulose coated slide and was obtained from Schleicher & Schuell. The slide shown as GAPS is coated with an amino-silane surface and was obtained from Corning®. Successful kinase assays according to the method provided herein have also been obtained using ZetaGrip slides (available from TeleChem International, Inc., ArrayIt™ Division, Sunnyvale, Calif.; on the Internet at www.arrayit.com).

### Safety Considerations

1. The operator must follow proper procedures and use cautions when handling radioactive materials.

2. Before using the microarrayer, the operator should be trained to avoid injuries to the person and/or damages to the machine.

Approximately fifty human protein kinases have been successfully employed in the methods provided in this Example. Validated kinases include a variety of kinases of direct relevance to disease, including Abl, EGFR, FGFR, members of the src kinase family and a variety of PKC isoforms. The methods provided herein are broadly applicable to all kinase families, as validated kinases represent all branches of the kinase phylogenetic tree of the human kinome.

### Example II

### Inhibitor Specificity Profiling

Fifty different kinases were immobilized on a slide together with a substrate as described above. A mixture of Myelin Basic Protein (MBP), histone and casein was used as substrate. The kinase reactions were performed in the presence of H89 inhibitor, Rottlerin inhibitor or PP2 inhibitor. The inhibitors were obtained from Calbiochem. The PP2 inhibitor is an inhibitor of tyrosine kinases. The concentration of inhibitor was 100 μm for each inhibitor. The control reaction was performed in the absence of inhibitor. The specificity of the assay was demonstrated by the fact that PP2 inhibitor strongly inhibited tyrosine kinases.

### Example III

### Dose-Response Analyses

Microarrays were prepared with 10 wells/slide, wherein the kinases EPHB3, FYN, and PRKCD and their substrate were immobilized in each well. The slide was coated with substrate essentially as described in Example I. Subsequently, a gasket with 10 openings was applied to the surface of the slide thereby creating 10 wells, i.e., the gasket provides the barriers between the wells. The accession numbers for the different kinases in the NCBI database are: for FYN: NM_{—}002037; for PRKCD: NM_{—}006254; and for EPHB3: NM_{—}004443. A mixture of Myelin Basic Protein (MBP), histone and casein was used as substrate. The kinase reaction was performed in each well with a different concentration of PP2 inhibitor.

The data show that PP2 strongly inhibits the tyrosine kinases FYN and EPHB3 but not the serine/threonine kinase PRKCD. In a second experiment, the kinase reaction was performed in each well with a different concentration of staurosporine. The dose-response curve demonstrates that staurosporine strongly inhibits PRKCD and FYN but not EPHB3.

### Example IV

### Comprehensive Inhibitor Assays

The present example provides a method for performing inhibitor assays using methods provided herein, and provides results obtained using those methods. The surface of a slide is coated with substrate within the wells of a multiwell array. The surface is coated with substrate, and washed and blocked as described in Example I. Subsequently, a gasket with openings is applied to the surface of the slide thereby creating wells, i.e., the gasket provides the barriers between the wells.

The kinases are deposited on the surface by the following procedure. The dimensions of the wells of the multi-well array used are obtained and the areas on the slides that will match the wells are defined. These numbers are used to calibrate the microarrayer so that the deposited spots will locate within the wells. The wells are formed later by placing the gasket with openings on top of the surface of the solid support.

The number of proteins that can be deposited per well depends on the dimension of the well and the spacing required. The chambers made by Scleicher&Schuell and Grace Bio-labs have 7000 μm×7000 μm wells and allow up to 12×12 spots/well deposited if the spacing is 500 μm. At least 4 replicate per kinase is recommended for quantitative experiments.

The plate of kinases to be deposited is made so that the printing pins pick up the identical kinase preparation (identical volume, concentration, buffer components, etc.) at the same time. This will ensure comparable results among the arrays. In addition, kinase activities should be assessed and normalized to give uniform signals within the array. The kinases are deposited onto the slide as described in Example I.

The kinase assay is performed by removing the plastic covering from sticky side of the chamber, placing the chamber carefully on the slides, aligning the wells to the deposited areas. The chamber is placed on the slide to make a tight seal between wells. Subsequently, the kinase assay buffer with gamma-AT³³P is prepared as described in Example I. Inhibitors (or other molecules of interest or concentrations of the same molecule) are prepared in aliquots. The cover slip is removed from the chamber, thereby exposing the wells. Appropriate amounts of inhibitor and kinase assay buffer is added to wells (volumes that will cover the well but not exceed the well capacity). The cover slip is placed on the slide and the entire slide/chamber assembly is placed in a 50 ml tube. The slides are incubated at 30° C. for 90 minutes, making sure the slides sit flat. The slides are washed as described in Example I. The chamber is removed from the tube using a pair of tweezers and the wash procedure is repeated once. The kinase reaction is evaluated as described in Example I.

### Example V

### Sequential Printing of Substrate and Enzyme

### Introduction

The following experiments were conducted to test whether sequential printing of substrate and enzyme affects the enzymatic reaction between the substrate and the enzyme on the surface of a solid support. The experiments were further conducted to test the effect of (i) the chemistry used for immobilizing substrate and enzyme on the surface of the solid support; and (ii) the effect of a washing step before printing of substrate and enzyme on the surface of a solid support on the signal-to-noise ratio of the enzymatic reaction between substrate and enzyme.

### Materials and Methods

Kinase substrates were deposited on the surface of a solid support as disclosed in Example I. Subsequently, kinases were deposited on the same spots as the kinase substrates. The kinase reaction was performed as described above in Example I. The kinases deposited on the array were Isoforms of PKC (including PKCh, PKCd, PKCi, and mixture), LCK, LYN, FYN, PKA. Some of the kinases used were obtained from commercial sources (PKC mixture, PKA, FYN, LYN, and LCK). Other kinases (PKC isoforms, FYN, LYN, and LCK) were produced by standard techniques. The substrate that was deposited was a Casein, Histone, MBP, and poly(GluTyr) mixture. Eight concentrations (2× dilutions; 250, 125, 62.5, 31.25, 15,6, 7.8, 3.9, 1.9 ug/ml for each substrate in the mixture) were used. Slides were washed in 40 ml of PBS in a 50 ml conical tube for 1-2 minutes, twice.

### Results

A detectable signal specific for the enzymatic reaction was obtained for each sample, except the FAST sample without washing. In other words, when FAST slides were used, a detectable signal was obtained only if the slide had been washed before the substrate and the kinase were deposited on the slide. However, when SuperAldehyde slides (TeleChem International, Inc.) or GAPS slides, respectively, were used, a washing step before printing of kinase and substrate improved the signal of the kinase reaction only slightly. Further, FAST slides gave the highest background and SuperAldehyde the lowest. Higher kinase concentrations gave higher signals on all three types of slides. In summary, the experiment illustrates that both the protein and the substrate can be deposited on the solid support in methods provided herein.

### Example VI

### Comparison of Microarray Assays where Enzymes and Substrates are Immobilized on a Solid Support Versus Conventional Solution Assays

To compare results obtained from microarray assay methods of the present invention to conventional solution assays, five kinases (ARG, FYN, PKCa, PKCd, and PKCe) were assayed using methods provided herein and compared to solution assays performed by a commercial service (Upstate, Waltham, Mass.) using PP2 (a tyrosine kinase specific inhibitor) at 1 μM. The kinase microarray assay with immobilized kinases and immobilized substrates was performed according to the method provided in Example I. The substrates, which included a mixture of 10 mg/ml of histone, casein, myelin basic protein (MBP), and poly-glutamic acid-tyrosine (polyEY), were coated on the surface of a glass slide.

The concentration of substrates that was used for coating slides was 10 μg/ml for each of the 4 substrates. SuperAldehyde slides from TeleChem International were used for the assay.

The percentage of inhibition data show an excellent agreement between the microarray assay of the present invention and the traditional solution-based assay. The microarray assays of the present invention provide significant advantages, as discussed herein. For example, the microarray assays of the present invention are performed with significantly less inhibitor and kinase than the solution assay. Furthermore, the microarray assay method of the present invention employ a solid-phase co-localization of kinase substrate pairs, enabling parallel processing of large numbers of kinases in a single reaction.

### Example VII

### Global Specificity Profiling Experiment

This example demonstrates that single point inhibition assays using methods provided herein, enable global evaluation of compound specificity. To assess the application of microarray assays for compound profiling, seven known inhibitors (see Table of inhibitors used in global specificity profiling experiment) and one control (2% DMSO) were tested on microarrays deposited with a group of kinases (as well as positive and negative controls). The method of Example I was used. Twelve spots of each kinase or control were deposited on each array, and three arrays were used for each inhibitor. A mixture of generic kinase substrates (histone, casein, MBP, and polyEY) was used in the assay. The average of all signals from the same inhibitor or control experiment was calculated.

The percentage-of-inhibition data for 39 kinases active on these substrates (activity>negative+2 standard deviations) obtained from this experiment were in agreement with published specificity data For example, the broad spectrum of kinases inhibited by staurosporine was clearly evident, while FYN (kinase 33) was inhibited only by PP2 (aside from staurosporine). The general specificities observed were consistent with the known general specificities for these inhibitors, which are listed in Table 3. For instance, PP2 primarily inhibited tyrosine kinases, while Ro-31-8220 more specifically targeted the serine-threonine kinases. The complete list of kinases analyzed in this experiment are provided in Table 4. To expedite data analysis regarding the kinase families that are inhibited by a particular substrate or group of substrates, a graphical representation can be constructed of inhibition data for substrates in such a manner that phylogenetically related kinases can be spatially arranged on the graphical representation.

TABLE 3
Inhibitors used in global specificity profiling experiment

| Name | General Specificity |
|---|---|
| H-89 | Serine/threonine |
| SB 202190 | Serine/threonine |
| Ro-31-8220 | Serine/threonine |
| Staurosporine | Broad |
| Genistein | Tyrosine |
| PP2 | Tyrosine |
| AG 490 | Tyrosine |

TABLE 4
Kinases used in the global specificity profiling experiment

| Kinase Number | Kinase Name |
|---|---|
| 1 | MAPK3K7 |
| 2 | AZK |
| 3 | ILK |
| 4 | BMPR1B |
| 5 | SYK |
| 6 | SYK |
| 7 | RET |
| 8 | LCK |
| 9 | LYN |
| 10 | BLK |
| 11 | FGR |
| 12 | FYN |
| 13 | FRK |
| 14 | EPHA3 |
| 15 | EPHA4 |
| 16 | STK3 |
| 17 | CAMK2D |
| 18 | NA |
| 19 | PIM2 |
| 20 | PIM1 |
| 21 | STK22 |
| 22 | TLK2 |
| 23 | MAPKAPK2 |
| 24 | CLK2 |
| 25 | DYRK1A |
| 26 | PCTAIRE |
| 27 | CDKL1 |
| 28 | MAPK8 |
| 29 | PRKCZ |
| 30 | PRKCI |
| 31 | PRKCH |
| 32 | PRKCE |
| 33 | PRKCD |
| 34 | PRKCL2 |
| 35 | MAST205 |
| 36 | ADRBK1 |
| 37 | VRK3 |
| 38 | STK16 |
| 39 | TBK1 |

### Example VIII

### Validation of Ic₅₀ Measurement Using Kinase Activity Microarrays of the Present Invention

The present Example illustrates that by measuring single-point inhibitions at varying inhibitor concentrations, kinase microarrays can be used to measure IC₅₀ values in a highly parallel fashion. The experiment was performed according to Example I, wherein various concentrations of staurosporine were included in the kinase assay buffer (i.e. the buffer included in the incubating step). Substrates for Protein kinase C_{delta} were coated on a series of ten slides, and subsequently Protein Kinase C_{delta} was deposited on the slides. Each slide contained 50 replicates of Protein Kinase C_{delta}. Substrates used to coat slides:

The same 4 substrates at 10 ug/ml each (casein, MBP, histone, pEY) as in Example VII were used. A Microarray printer from GeneMachines™, made by Genomic Solutions was used for printing the arrays. Accordingly, both substrate and Protein Kinase C_{delta} were immobilized on the slide. An IC₅₀ of 1 nM was calculated using the methods provided herein, in good agreement with the literature value of 0.7 nM. Accordingly, methods of the present invention can be used to calculate IC₅₀ values for inhibitors.

### Example IX

### Further Analysis of a Plurality of Inhibitors and a Plurality of Kinases

The present Example provides experiments that illustrate that the methods provided herein are effective for many types of kinases and can be used to analyze various test molecules. The assays were performed essentially as disclosed in Example I. A large number of kinases and enzymes were analyzed (see Table 5, Parts I and II). The following tables summarize qualitatively the inhibition by the inhibitors. Inhibitors showed different potency and specificity, as expected for this type of assay.

TABLE 5
(Part I) Inhibition Results

| | | | | | | | | | | | Rottler- | | | |
|---|---|---|---|---|---|---|---|---|---|---|---|---|---|---|
| | | | | | | | | Solu- | | | in | | | |
| Sub- | Sub- | | | | | | GST_{—} | tion_{—} | Micro- | | (Mallo- | Quer- | SB | |
| array_{—} | array_{—} | Plate_{—} | Plate_{—} | Plate_{—} | | Do- | Expres- | Activ- | array_{—} | H-89 | toxin) | cetin | 202190 | KN-62 |
| Row | Column | Block | Row1 | Column1 | Name | main | sion | ity | Activity | 100 uM | 100 uM | 100 uM | 100 uM | 100 uM |
| 1 | 1 | 1 | A | 12 | RAF1 | 0 | 1 | 2 | 0 | | | | | |
| 1 | 1 | 2 | C | 12 | LOC51231 | 1 | 1 | 3 | 2 | 2 | 0 | 0 | | |
| 1 | 1 | 3 | E | 12 | Homo | 0 | 1 | 3 | 2 | 1 | 1 | 0 | | |
| | | | | | sapiens, [symbol] | | | | | | | | | |
| 1 | 1 | 4 | G | 12 | EGFR | 1 | 1 | 2 | 0 | | | | | |
| 1 | 1 | 5 | A | 6 | LCK | 0 | 1 | 3 | 2 | 2 | 2 | 0 | | |
| 1 | 1 | 6 | C | 6 | STK6 | 1 | 1 | 3 | 1 | 0 | 2 | 0 | | |
| 1 | 1 | 7 | E | 6 | MAP2K4 | 1 | 1 | 3 | 1 | 1 | 1 | 0 | | |
| 1 | 1 | 8 | G | 6 | DYRK1A | 1 | 1 | 2.5 | 2 | 2 | 0 | 0 | | |
| 2 | 1 | 1 | A | 11 | MAP3K2 | 1 | 1 | 3 | 1 | | | | | |
| 2 | 1 | 2 | C | 11 | JIK | 1 | 1 | 3 | 0 | | | | | |
| 2 | 1 | 3 | E | 11 | Homo | 0 | 1 | 3 | 1 | 1 | 2 | 1 | | |
| | | | | | sapiens, [symbol] | | | | | | | | | |
| 2 | 1 | 4 | G | 11 | STK13 | 1 | 1 | 3 | 1 | 1 | 1 | 2 | | |
| 2 | 1 | 5 | A | 5 | LYN | 0 | 1 | 3 | 2 | 2 | 2 | 2 | | |
| 2 | 1 | 6 | C | 5 | MAP4K3 | 1 | 0 | 3 | 0 | 1 | 1 | 1 | | |
| 2 | 1 | 7 | E | 5 | EPHB2 | 1 | 1 | 3 | 2 | 2 | 2 | 2 | | |
| 2 | 1 | 8 | G | 5 | PTK2B | 1 | 1 | 3 | 1 | 1 | 2 | 1 | | |
| 3 | 1 | 1 | A | 10 | CAMK1 | 1 | 1 | 3 | 0 | | | | | |
| 3 | 1 | 2 | C | 10 | STK38 | 1 | 0 | 2 | 0 | | | | | |
| 3 | 1 | 3 | E | 10 | Homo | 0 | 1 | 3 | 0 | | | | | |
| | | | | | sapiens, [symbol] | | | | | | | | | |
| 3 | 1 | 4 | G | 10 | MAST205 | 1 | 1 | 2.5 | 0 | | | | | |
| 3 | 1 | 5 | A | 4 | FYN | 0 | 1 | 3 | 2 | 2 | 2 | 0 | | |
| 3 | 1 | 6 | C | 4 | MARK2 | 1 | 1 | 3 | 2 | 2 | 2 | 0 | | |
| 3 | 1 | 7 | E | 4 | ITK | 0 | 1 | 3 | 1 | 1 | 1 | 0 | | |
| 3 | 1 | 8 | G | 4 | PINK1 | 1 | 1 | 3 | 0 | | | | | |
| 4 | 1 | 1 | A | 9 | MET | 1 | 1 | 3 | 0 | | | | | |
| 4 | 1 | 2 | C | 9 | IRAK3 | 1 | 1 | 2 | 2 | 0 | 2 | 0 | | |
| 4 | 1 | 3 | E | 9 | Homo | 0 | 1 | 2 | 2 | 2 | 2 | 0 | | |
| | | | | | sapiens, [symbol] | | | | | | | | | |
| 4 | 1 | 4 | G | 9 | MAP3K7 | 0 | 1 | 3 | 2 | 2 | 2 | 0 | | |
| 4 | 1 | 5 | A | 3 | PCTK1 | 0 | 0 | 3 | 2 | 2 | 0 | 0 | | |
| 4 | 1 | 6 | C | 3 | STK16 | 1 | 1 | 3 | 2 | 2 | 0 | 0 | | |
| 4 | 1 | 7 | E | 3 | CLK1 | 1 | 1 | 3 | 1 | | | | | |
| 4 | 1 | 8 | G | 3 | SRC | 1 | 1 | 3 | 0 | | | | | |
| 5 | 1 | 1 | A | 8 | LYN | 1 | 1 | 3 | 0 | | | | | |
| 5 | 1 | 2 | C | 8 | TLK2 | 1 | 1 | 3 | 2 | 2 | 2 | 0 | | |
| 5 | 1 | 3 | E | 8 | PAK1 | 1 | 1 | 3 | 0 | | | | | |
| 5 | 1 | 4 | G | 8 | FGFR2 | 1 | 1 | 3 | 2 | 2 | 2 | 0 | | |
| 5 | 1 | 5 | A | 2 | PRKCI | 0 | 1 | 3 | 2 | 2 | 2 | 0 | | |
| 5 | 1 | 6 | C | 2 | PIM1 | 1 | 1 | 3 | 2 | 2 | 2 | 0 | | |
| 5 | 1 | 7 | E | 2 | SRPK1 | 1 | 1 | 3 | 0 | | | | | |
| 5 | 1 | 8 | G | 2 | FLJ20574 | 0 | 1 | 3 | 0 | | | | | |
| 6 | 1 | 1 | A | 7 | FYN | 1 | 1 | 3 | 0 | | | | | |
| 6 | 1 | 2 | C | 7 | TGFBR2 | 1 | 1 | 2 | 2 | 2 | 2 | 0 | | |
| 6 | 1 | 3 | E | 7 | MAP3K4 | 1 | 1 | 3 | 1 | 1 | 1 | 0 | | |
| 6 | 1 | 4 | G | 7 | TOPK | 1 | 1 | 3 | 2 | 2 | 2 | 0 | | |
| 6 | 1 | 5 | A | 1 | MAP3K3 | 0 | 1 | 3 | 2 | 2 | 2 | 0 | | |
| 6 | 1 | 6 | C | 1 | ADRBK1 | 1 | 1 | 3 | 0 | | | | | |
| 6 | 1 | 7 | E | 1 | MAPKAPK3 | 1 | 1 | 3 | 0 | | | | | |
| 6 | 1 | 8 | G | 1 | TBK1 | 0 | 1 | 3 | 2 | 2 | 1 | 0 | | |

(Part I) Inhibition Results

| | | | | | | | | | | | | AG | AG | AG |
|---|---|---|---|---|---|---|---|---|---|---|---|---|---|---|
| Sub- | Sub- | | | | | Ro-31- | Ro-31- | Stauro- | Genis- | Genis- | PP2 | 490 | 1296 | 1478 |
| array_{—} | array_{—} | Plate_{—} | Plate_{—} | Plate_{—} | | 8220 | 8220 | sporine | tein | tein <3.7 | 100 | 100 | 100 | 100 |
| Row | Column | Block | Row1 | Column1 | Name | 100 uM | 90 uM | 21.5 uM | 100 uM | mM | uM | uM | uM | uM |
| 1 | 1 | 1 | A | 12 | RAF1 | | | | | | | | | |
| 1 | 1 | 2 | C | 12 | LOC51231 | 2 | 0 | 0 | 2 | 0 | 0 | 0 | | 0 |
| 1 | 1 | 3 | E | 12 | Homo | 1 | | | 2 | | 2 | 0 | | 1 |
| | | | | | sapiens, [symbol] | | | | | | | | | |
| 1 | 1 | 4 | G | 12 | EGFR | | | | | | | | | |
| 1 | 1 | 5 | A | 6 | LCK | 2 | 0 | 0 | 2 | 0 | 2 | 2 | | 2 |
| 1 | 1 | 6 | C | 6 | STK6 | 2 | 0 | 0 | 2 | 0 | 2 | 2 | | 0 |
| 1 | 1 | 7 | E | 6 | MAP2K4 | 1 | 0 | 0 | 2 | 0 | 2 | 0 | | 0 |
| 1 | 1 | 8 | G | 6 | DYRK1A | 2 | 0 | 0 | 2 | 1 | 1 | 2 | | 0 |
| 2 | 1 | 1 | A | 11 | MAP3K2 | | | | | | | | | |
| 2 | 1 | 2 | C | 11 | JIK | | | | | | | | | |
| 2 | 1 | 3 | E | 11 | Homo | 2 | | | 2 | | 2 | 2 | | 2 |
| | | | | | sapiens, [symbol] | | | | | | | | | |
| 2 | 1 | 4 | G | 11 | STK13 | 2 | | | 2 | | 2 | 2 | | 1 |
| 2 | 1 | 5 | A | 5 | LYN | 2 | 0 | 0 | 2 | 2 | 2 | 2 | | 2 |
| 2 | 1 | 6 | C | 5 | MAP4K3 | 2 | | | 2 | | 2 | 1 | | 2 |
| 2 | 1 | 7 | E | 5 | EPHB2 | 2 | 0 | 0 | 2 | 0 | 2 | 2 | | 2 |
| 2 | 1 | 8 | G | 5 | PTK2B | 2 | 0 | 0 | 2 | | 2 | 2 | | 2 |
| 3 | 1 | 1 | A | 10 | CAMK1 | | | | | | | | | |
| 3 | 1 | 2 | C | 10 | STK38 | | | | | | | | | |
| 3 | 1 | 3 | E | 10 | Homo | | | | | | | | | |
| | | | | | sapiens, [symbol] | | | | | | | | | |
| 3 | 1 | 4 | G | 10 | MAST205 | | | | | | | | | |
| 3 | 1 | 5 | A | 4 | FYN | 2 | 0 | 2 | 2 | 1 | 2 | 2 | | 2 |
| 3 | 1 | 6 | C | 4 | MARK2 | 2 | | | 2 | | 2 | 2 | | 2 |
| 3 | 1 | 7 | E | 4 | ITK | 2 | | | 2 | | 2 | 1 | | 1 |
| 3 | 1 | 8 | G | 4 | PINK1 | | | | | | | | | |
| 4 | 1 | 1 | A | 9 | MET | | | | | | | | | |
| 4 | 1 | 2 | C | 9 | IRAK3 | 2 | | | 2 | | 2 | 0 | | 2 |
| 4 | 1 | 3 | E | 9 | Homo | 0 | | | 2 | | 2 | 0 | | 0 |
| | | | | | sapiens, [symbol] | | | | | | | | | |
| 4 | 1 | 4 | G | 9 | MAP3K7 | 2 | 2 | 2 | 1 | 0 | 2 | 0 | | 0 |
| 4 | 1 | 5 | A | 3 | PCTK1 | 0 | | | 1 | | 1 | 0 | | 0 |
| 4 | 1 | 6 | C | 3 | STK16 | 1 | 2 | 2 | 0 | 0 | 2 | 0 | | 0 |
| 4 | 1 | 7 | E | 3 | CLK1 | | | | | | | | | |
| 4 | 1 | 8 | G | 3 | SRC | | | | | | | | | |
| 5 | 1 | 1 | A | 8 | LYN | | | | | | | | | |
| 5 | 1 | 2 | C | 8 | TLK2 | 1 | 2 | 2 | 2 | 0 | 2 | 2 | | 2 |
| 5 | 1 | 3 | E | 8 | PAK1 | | | | | | | | | |
| 5 | 1 | 4 | G | 8 | FGFR2 | 2 | 2 | 2 | 2 | 0 | 2 | 2 | | 2 |
| 5 | 1 | 5 | A | 2 | PRKCI | 0 | 2 | 2 | 2 | 0 | 1 | 2 | | 0 |
| 5 | 1 | 6 | C | 2 | PIM1 | 0 | 2 | 2 | 2 | 0 | 2 | 2 | | 1 |
| 5 | 1 | 7 | E | 2 | SRPK1 | | | | | | | | | |
| 5 | 1 | 8 | G | 2 | FLJ20574 | | | | | | | | | |
| 6 | 1 | 1 | A | 7 | FYN | | | | | | | | | |
| 6 | 1 | 2 | C | 7 | TGFBR2 | 2 | 2 | 2 | 2 | 1 | 2 | 2 | | 2 |
| 6 | 1 | 3 | E | 7 | MAP3K4 | 2 | | | 1 | | 2 | 2 | | 1 |
| 6 | 1 | 4 | G | 7 | TOPK | 0 | 2 | 2 | 2 | 0 | 2 | 2 | | 1 |
| 6 | 1 | 5 | A | 1 | MAP3K3 | 0 | 2 | 2 | 2 | 2 | 2 | 2 | | 2 |
| 6 | 1 | 6 | C | 1 | ADRBK1 | | | | | | | | | |
| 6 | 1 | 7 | E | 1 | MAPKAPK3 | | | | | | | | | |
| 6 | 1 | 8 | G | 1 | TBK1 | 2 | | | 2 | | 1 | 2 | | 2 |

(Part II) Inhibition Results

| | | | | | | | | | | | Rottler- | | | |
|---|---|---|---|---|---|---|---|---|---|---|---|---|---|---|
| | | | | | | | | Solu- | | | in | | | |
| Sub- | Sub- | | | | | | GST_{—} | tion_{—} | Micro- | | (Mallo- | Quer- | SB | |
| array_{—} | array_{—} | Plate_{—} | Plate_{—} | Plate_{—} | | Do- | Expres- | Activ- | array_{—} | H-89 | toxin) | cetin | 202190 | KN-62 |
| Row | Column | Block | Row2 | Column2 | Name | main | sion | ity | Activity | 100 uM | 100 uM | 100 uM | 100 uM | 100 uM |
| 1 | 2 | 1 | B | 12 | BMX | 1 | 1 | 3 | 2 | 1 | 1 | 0 | | |
| 1 | 2 | 2 | D | 12 | FGFR1 | 1 | 1 | 3 | 2 | 1 | 1 | 0 | | |
| 1 | 2 | 3 | F | 12 | CDKL3 | 0 | 1 | 2 | 1 | 1 | 1 | 0 | | |
| 1 | 2 | 4 | H | 12 | Empty | 0 | 0 | | 0 | | | | | |
| 1 | 2 | 5 | B | 6 | ABL1 | 1 | 1 | 3 | 2 | 0 | 0 | 0 | | |
| 1 | 2 | 6 | D | 6 | STK4 | 1 | 1 | 3 | 2 | 2 | 0 | 0 | | |
| 1 | 2 | 7 | F | 6 | Homo | 0 | 1 | 2 | 2 | 2 | 0 | 0 | | |
| | | | | | sapiens, [symbol] | | | | | | | | | |
| 1 | 2 | 8 | H | 6 | PRKG1 | 1 | 1 | 2 | 0 | | | | | |
| 2 | 2 | 1 | B | 11 | MERTK | 1 | 1 | 3 | 2 | 2 | 2 | 1 | | |
| 2 | 2 | 2 | D | 11 | DYRK2 | 1 | 1 | 3 | 2 | 2 | 2 | 2 | | |
| 2 | 2 | 3 | F | 11 | STK24 | 0 | 1 | 3 | 2 | 2 | 1 | 1 | | |
| 2 | 2 | 4 | H | 11 | Empty | 0 | 0 | | 0 | | | | | |
| 2 | 2 | 5 | B | 5 | EPHB3 | 1 | 1 | 3 | 2 | 1 | 2 | 2 | | |
| 2 | 2 | 6 | D | 5 | TTK | 1 | 1 | 3 | 0 | | | | | |
| 2 | 2 | 7 | F | 5 | Homo | 0 | 1 | 2 | 2 | 1 | 0 | 2 | | |
| | | | | | sapiens, [symbol] | | | | | | | | | |
| 2 | 2 | 8 | H | 5 | FER | 1 | 1 | 3 | 2 | 2 | 2 | 1 | | |
| 3 | 2 | 1 | B | 10 | FGR | 0 | 1 | 3 | 2 | 2 | 2 | 2 | | |
| 3 | 2 | 2 | D | 10 | CDK10 | 0 | 1 | 2 | 0 | | | | | |
| 3 | 2 | 3 | F | 10 | Homo | 0 | 1 | 3 | 2 | 2 | 2 | 2 | | |
| | | | | | sapiens, [symbol] | | | | | | | | | |
| 3 | 2 | 4 | H | 10 | GST | 0 | 1 | | 0 | | | | | |
| 3 | 2 | 5 | B | 4 | FGFR2 | 1 | 1 | 3 | 2 | 2 | 0 | 0 | | |
| 3 | 2 | 6 | D | 4 | PAK3 | 1 | 1 | 3 | 2 | 2 | 0 | 2 | | |
| 3 | 2 | 7 | F | 4 | Homo | 0 | 0 | 3 | 1 | 2 | 0 | 1 | | |
| | | | | | sapiens, [symbol] | | | | | | | | | |
| 3 | 2 | 8 | H | 4 | RIPK1 | 1 | 0 | 3 | 0 | | | | | |
| 4 | 2 | 1 | B | 9 | TEK | 1 | 1 | 3 | 0 | | | | | |
| 4 | 2 | 2 | D | 9 | CSNK2A1 | 0 | 1 | 3 | 2 | 0 | 2 | 0 | | |
| 4 | 2 | 3 | F | 9 | Homo | 0 | 1 | 3 | 2 | 2 | 0 | 0 | | |
| | | | | | sapiens, [symbol] | | | | | | | | | |
| 4 | 2 | 4 | H | 9 | GST | 0 | 1 | | 0 | | | | | |
| 4 | 2 | 5 | B | 3 | PRKCH | 0 | 1 | 3 | 2 | 2 | 2 | 0 | | |
| 4 | 2 | 6 | D | 3 | PAK4 | 1 | 1 | 2 | 0 | | | | | |
| 4 | 2 | 7 | F | 3 | Homo | 0 | 1 | 2 | 2 | 2 | 0 | 0 | | |
| | | | | | sapiens, [symbol] | | | | | | | | | |
| 4 | 2 | 8 | H | 3 | DAPK2 | 1 | 1 | 3 | 2 | 2 | 1 | 0 | | |
| 5 | 2 | 1 | B | 8 | EPHB1 | 1 | 1 | 3 | 2 | 1 | 1 | 0 | | |
| 5 | 2 | 2 | D | 8 | EPHA3 | 1 | 1 | 2 | 0 | | | | | |
| 5 | 2 | 3 | F | 8 | Homo | 0 | 1 | 3 | 2 | 1 | 0 | 0 | | |
| | | | | | sapiens, [symbol] | | | | | | | | | |
| 5 | 2 | 4 | H | 8 | Cell | 0 | 0 | | 0 | | | | | |
| 5 | 2 | 5 | B | 2 | PRKCD | 0 | 1 | 3 | 2 | 1 | 0 | 0 | | |
| 5 | 2 | 6 | D | 2 | LOC57118 | 1 | 1 | 3 | 1 | 1 | 0 | 0 | | |
| 5 | 2 | 7 | F | 2 | Homo | 0 | 1 | 2 | 2 | 0 | 2 | 0 | | |
| | | | | | sapiens, [symbol] | | | | | | | | | |
| 5 | 2 | 8 | H | 2 | FLJ20574 | 1 | 1 | 3 | 1 | | | | | |
| 6 | 2 | 1 | B | 7 | RET | 1 | 1 | 3 | 2 | 0 | 0 | 0 | | |
| 6 | 2 | 2 | D | 7 | ACVR1B | 1 | 1 | 3 | 0 | | | | | |
| 6 | 2 | 3 | F | 7 | Homo | 0 | 1 | 3 | 2 | 0 | 2 | 0 | | |
| | | | | | sapiens, [symbol] | | | | | | | | | |
| 6 | 2 | 4 | H | 7 | Cell | 0 | 0 | | 0 | | | | | |
| 6 | 2 | 5 | B | 1 | CAMK2D | 1 | 1 | 3 | 2 | 0 | 2 | 0 | | |
| 6 | 2 | 6 | D | 1 | MKNK2 | 1 | 1 | 3 | 0 | | | | | |
| 6 | 2 | 7 | F | 1 | Homo | 0 | 1 | 3 | 2 | 0 | 1 | 0 | | |
| | | | | | sapiens, [symbol] | | | | | | | | | |
| 6 | 2 | 8 | H | 1 | PHKG1 | 1 | 0 | 3 | 0 | | | | | |

(Part II) Inhibition Results

| | | | | | | | | | | | | AG | AG | AG |
|---|---|---|---|---|---|---|---|---|---|---|---|---|---|---|
| Sub- | Sub- | | | | | Ro-31- | | | Genis- | | PP2 | 490 | 1296 | 1478 |
| array_{—} | array_{—} | Plate_{—} | Plate_{—} | Plate_{—} | | 8220 | Ro-31- | Stauro- | tein | Genis- | 100 | 100 | 100 | 100 |
| Row | Column | Block | Row2 | Column2 | Name | 100 uM | 8220 | sporine | 100 uM | tein | uM | uM | uM | uM |
| 1 | 2 | 1 | B | 12 | BMX | 2 | | | 0 | | 2 | 0 | | 1 |
| 1 | 2 | 2 | D | 12 | FGFR1 | 2 | | | 0 | | 2 | 0 | | 1 |
| 1 | 2 | 3 | F | 12 | CDKL3 | 2 | | | 0 | | 2 | 2 | | 1 |
| 1 | 2 | 4 | H | 12 | Empty | | | | | | | | | |
| 1 | 2 | 5 | B | 6 | ABL1 | 2 | 0 | 0 | 0 | 0 | 2 | 0 | | 1 |
| 1 | 2 | 6 | D | 6 | STK4 | 2 | 0 | 0 | 0 | 0 | 2 | 0 | | 1 |
| 1 | 2 | 7 | F | 6 | Homo | 2 | 0 | 0 | 0 | 0 | 2 | 0 | | 1 |
| | | | | | sapiens, [symbol] | | | | | | | | | |
| 1 | 2 | 8 | H | 6 | PRKG1 | | | | | | | | | |
| 2 | 2 | 1 | B | 11 | MERTK | 2 | | | 2 | | 2 | 2 | | 2 |
| 2 | 2 | 2 | D | 11 | DYRK2 | 2 | 0 | 0 | 2 | 1 | 2 | 2 | | 2 |
| 2 | 2 | 3 | F | 11 | STK24 | 2 | | | 2 | | 2 | 2 | | 1 |
| 2 | 2 | 4 | H | 11 | Empty | | | | | | | | | |
| 2 | 2 | 5 | B | 5 | EPHB3 | 2 | 0 | 0 | 2 | 0 | 2 | 2 | | 2 |
| 2 | 2 | 6 | D | 5 | TTK | | | | | | | | | |
| 2 | 2 | 7 | F | 5 | Homo | 2 | 0 | 0 | 2 | 0 | 2 | 2 | | 2 |
| | | | | | sapiens, [symbol] | | | | | | | | | |
| 2 | 2 | 8 | H | 5 | FER | 2 | | | 2 | | 2 | 2 | | 1 |
| 3 | 2 | 1 | B | 10 | FGR | 2 | 2 | 2 | 2 | 2 | 2 | 2 | | 2 |
| 3 | 2 | 2 | D | 10 | CDK10 | | | | | | | | | |
| 3 | 2 | 3 | F | 10 | Homo | 2 | 2 | 2 | 2 | 2 | 2 | 2 | | 2 |
| | | | | | sapiens, [symbol] | | | | | | | | | |
| 3 | 2 | 4 | H | 10 | GST | | | | | | | | | |
| 3 | 2 | 5 | B | 4 | FGFR2 | 2 | 0 | 2 | 2 | 1 | 2 | 2 | | 2 |
| 3 | 2 | 6 | D | 4 | PAK3 | 2 | 2 | 2 | 2 | 0 | 2 | 1 | | 2 |
| 3 | 2 | 7 | F | 4 | Homo | 2 | | | 2 | | 2 | 2 | | 2 |
| | | | | | sapiens, [symbol] | | | | | | | | | |
| 3 | 2 | 8 | H | 4 | RIPK1 | | | | | | | | | |
| 4 | 2 | 1 | B | 9 | TEK | | | | | | | | | |
| 4 | 2 | 2 | D | 9 | CSNK2A1 | 2 | 0 | 2 | 2 | 0 | 2 | 2 | | 2 |
| 4 | 2 | 3 | F | 9 | Homo | 0 | 2 | 2 | 1 | 0 | 2 | 1 | | 2 |
| | | | | | sapiens, [symbol] | | | | | | | | | |
| 4 | 2 | 4 | H | 9 | GST | | | | | | | | | |
| 4 | 2 | 5 | B | 3 | PRKCH | 2 | 2 | 2 | 2 | 0 | 2 | 2 | | 0 |
| 4 | 2 | 6 | D | 3 | PAK4 | | | | | | | | | |
| 4 | 2 | 7 | F | 3 | Homo | 2 | 2 | 2 | 1 | 0 | 2 | 0 | | 2 |
| | | | | | sapiens, [symbol] | | | | | | | | | |
| 4 | 2 | 8 | H | 3 | DAPK2 | 2 | 2 | 2 | 2 | 0 | 2 | 2 | | 0 |
| 5 | 2 | 1 | B | 8 | EPHB1 | 0 | 2 | 2 | 1 | 0 | 2 | 0 | | 2 |
| 5 | 2 | 2 | D | 8 | EPHA3 | | | | | | | | | |
| 5 | 2 | 3 | F | 8 | Homo | 0 | 2 | 2 | 0 | 0 | 0 | 0 | | 0 |
| | | | | | sapiens, [symbol] | | | | | | | | | |
| 5 | 2 | 4 | H | 8 | Cell | | | | | | | | | |
| 5 | 2 | 5 | B | 2 | PRKCD | 0 | 2 | 2 | 0 | 0 | 0 | 0 | | 0 |
| 5 | 2 | 6 | D | 2 | LOC57118 | 0 | | | 0 | | 0 | 1 | | 0 |
| 5 | 2 | 7 | F | 2 | Homo | 0 | 2 | 2 | 2 | 0 | 2 | 2 | | 2 |
| | | | | | sapiens, [symbol] | | | | | | | | | |
| 5 | 2 | 8 | H | 2 | FLJ20574 | | | | | | | | | |
| 6 | 2 | 1 | B | 7 | RET | 0 | 2 | 2 | 2 | 0 | 2 | 2 | | 2 |
| 6 | 2 | 2 | D | 7 | ACVR1B | | | | | | | | | |
| 6 | 2 | 3 | F | 7 | Homo | 0 | 2 | 2 | 2 | 0 | 2 | 2 | | 2 |
| | | | | | sapiens, [symbol] | | | | | | | | | |
| 6 | 2 | 4 | H | 7 | Cell | | | | | | | | | |
| 6 | 2 | 5 | B | 1 | CAMK2D | 0 | 2 | 2 | 2 | 0 | 2 | 2 | | 2 |
| 6 | 2 | 6 | D | 1 | MKNK2 | | | | | | | | | |
| 6 | 2 | 7 | F | 1 | Homo | 0 | 0 | 2 | 1 | 0 | 1 | 2 | | 1 |
| | | | | | sapiens, [symbol] | | | | | | | | | |
| 6 | 2 | 8 | H | 1 | PHKG1 | | | | | | | | | |

Level 0 means no inhibition or unclear.
Level 1 means little or marginal inhibition.
Level 2 means substantial inhibition. [symbol]
indicates data missing or illegible when filed

### Example X

### Kinase Assay Using Mbp Substrate

In this illustrative example, four-well slides were designed with a hydrophobic mask surrounding 4-wells of aldehyde- or epoxy-coated glass (smooth or ES grade; Erie Scientific (Portsmouth, N.H.)). Additional slides used include aldehyde (#C60-5590-M20) or epoxy (#C50-5588-M20) smooth glass or aldehyde (#C62-5591-M20) or epoxy (#C52-5589-M20) ES glass slides from Erie Scientific, or aldehyde (#SMABC) or epoxy (#SMEBC) slides from Telechem International (Sunnyvale, Calif.). Bovine, dephosphorylated, Myelin Basic Protein (MBP) was purchased from Upstate Biotechnology (#13-110). MBP was diluted to 1 mg/ml in PBS, applied to the slide surface, covered with a coverslip, and left overnight at 4° C. to coat the slide with the MBP. Slides were washed 3 times with water and spun dry before printing.

Kinases were purchased from Panvera (Invitrogen, Carlsbad, Calif.), diluted in printing buffer (50 mM Tris pH 7.5, 25% glyercol, 0.05% TritonX-100, 2 mM DTT) and deposited using a GeneMachine OmniGrid100. Slides were stored at −20° C.

Reactions were performed following removal of the slide from the freezer. Reaction buffer (20 mM HEPES pH 7.5, 4 mM MgCl₂, 2 mM DTT, 20 uM ATP, 5% DMSO) was added with or without inhibitor, a coverslip applied, and the slide placed at 30° C. for the appropriate reaction time. The slide was washed with water to stop the reaction (3 times) and spun dry. ProQ Diamond Microarray Stain (Invitrogen #P33706) was applied, covered with a coverslip, and the slide was incubated in the dark at room temperature for 30 minutes. The slide was destained and washed three times with water, and spun dry. Results were acquired and analyzed using fluorometer (GenePix 4000B) and are summarized in Table 6.

TABLE 6

| | | | Panvera/ |
|---|---|---|---|
| | | MBP | Invitrogen Cat. |
| Receptor Tyrosine | | Kinase | No. (Carlsbad, |
| Kinase | Tag | Activity | CA) |
| AXL | C-terminal His | NT* | PV3971 |
| CSF1R | C-terminal His | Strong | PV3249 |
| EGFR | None | Negative | P2628 |
| EPHA1 | N-terminal GST | Strong | PV3841 |
| EPHA2 | N-terminal GST | Weak | PV3688 |
| EPHA3 | C-terminal His | Strong | PV3359 |
| EPHA4 | N-terminal GST | Strong | PV3651 |
| EPHA5 | N-terminal GST | Negative | PV3840 |
| EPHA7 | N-terminal GST | Weak | PV3689 |
| EPHA8 | N-terminal GST | Weak | PV3844 |
| EPHB1 | N-terminal GST | Strong | PV3786 |
| EPHB2 | N-terminal GST | Strong | PV3625 |
| EPHB3 | N-terminal GST | Strong | PV3658 |
| EPHB4 | C-terminal His | Strong | PV3251 |
| ERBB2 | C-terminal His | Negative | PV3366 |
| ERBB4 | N-terminal GST | Negative | PV3626 |
| DDR2 | | NT | PV3870 |
| FGFR1 | C-terminal His | Strong | PV3146 |
| FGFR2 | C-terminal His | Strong | PV3368 |
| FGFR3 | N-terminal His | Strong | PV3145 |
| FGFR4 | N-terminal His | Strong | P3054 |
| FLT1(VEGFR1) | N-terminal GST | Strong | PV3666 |
| FLT3 | C-terminal His | Strong | PV3182 |
| IGF1R | C-terminal His | Strong | PV3250 |
| INSR | N-terminal His | Strong | PV3664 |
| INSR | N-terminal GST | Strong | PV3781 |
| INSRR | N-terminal GST | Negative | PV3808 |
| KDR(VEGFR2) | C-terminal His | Strong | PV3660 |
| KIT | N-terminal His | Negative | P3081 |
| MERTK | N-terminal GST | Strong | PV3627 |
| MET | N-terminal His | Strong | PV3143 |
| MUSK | N-terminal GST | Negative | PV3834 |
| NTRK1 | C-terminal His | Strong | PV3144 |
| NTRK2 | C-terminal His | Strong | PV3616 |
| NTRK3 | C-terminal His | Strong | PV3617 |
| PDGFRA | N-terminal GST | Strong | PV3811 |
| PDGFRA, T674I | N-terminal GST | Negative | PV3847 |
| PDGFRB | N-terminal His | Strong | P3082 |
| RET | N-terminal GST | Strong | PV3819 |
| ROS1 | N-terminal GST | Strong | PV3814 |
| TIE2 | | NT | PV3628 |
| TYRO3 | N-terminal GST | Strong | PV3828 |

| | | MBP | Panvera/ |
|---|---|---|---|
| | | Kinase | Invitrogen Cat. |
| | Tag | Activity | No. |
| Cytoplasmic Tyrosine | | | |
| Kinase | | | |
| ABL1 | C-terminal His | Weak | P3049 |
| ABL2(ARG) | C-terminal His | Weak | PV3266 |
| ALK | | NT | PV3867 |
| BLK | N-terminal His | Weak | PV3683 |
| BMX | C-terminal His | Strong | PV3371 |
| BTK | C-terminal His | Strong | PV3363 |
| CSK | C-terminal His | Negative | P2927 |
| FER | N-terminal GST | Negative | PV3806 |
| FES | C-terminal His | Negative | PV3354 |
| FGR | C-terminal His | Weak | P3041 |
| FYN | C-terminal His | Strong | P3042 |
| FRK | | NT | PV3874 |
| HCK | C-terminal His | Strong | P2908 |
| ITK | | NT | PV3875 |
| JAK3 | N-terminal GST | Strong | PV3855 |
| LCK | C-terminal His | Strong | P3043 |
| LYNA | C-terminal His | Weak | P2906 |
| LYNB | C-terminal His | NT | P2907 |
| MATK(HYL) | C-terminal His | Negative | PV3370 |
| PTK2(FAK) | N-terminal GST | Negative | PV3832 |
| PTK6(BRK) | C-terminal His | Strong | PV3291 |
| SRC | C-terminal His | Strong | P3044 |
| SRCN1 | none | NT | P2904 |
| SRCN2 | none | NT | P2909 |
| SYK | N-terminal GST | Negative | PV3857 |
| TEC | C-terminal His | Negative | PV3269 |
| YES1 | C-terminal His | Weak | P3078 |
| ZAP70 | C-terminal His | Negative | P2782(20 ug) |
| Serine/Threonin | | | |
| Kinase | | | |
| ADRBK1(GRK2) | C-terminal His | Negative | PV3361 |
| ADRBK2(GRK3) | N-terminal GST | Negative | PV3827 |
| AKT1 | N-terminal His | Strong | P2999 |
| AKT2 | N-terminal His | Strong | PV3184 |
| AKT3 | N-terminal His | Strong | PV3185 |
| STK6(AuroraA) | N-terminal His | Weak | PV3612 |
| AURKB (AuroraB) | | NT | PV3970 |
| AURKC (AuroraC) | | NT | PV3856 |
| CAMK1D | N-terminal His | NT | PV3663 |
| CAMK2A | C-terminal His | NT | PV3142 |
| CAMK2D | C-terminal His | NT | PV3373 |
| CAMK4 | N-terminal GST | NT | PV3310 |
| CDK1/cyclin B | C-terminal His | Strong | PV3292 |
| CDK2/cyclin A | N-terminal His | Strong | PV3267 |
| CDK5/p35 | N-terminal His | Strong | PV3000 |
| CDK7/cyclinH | | NT | PV3868 |
| CHEK1 | N-terminal His | Strong | P3040 |
| CHEK2 | C-terminal His | Strong | PV3367 |
| CLK1 | N-terminal GST | Strong | PV3315 |
| CLK3 | N-terminal GST | Strong | PV3826 |
| CLK4 | N-terminal GST | Negative | PV3839 |
| CSNK1A1(CK1alpha1) | | NT | PV3850 |
| CSNK1D(CK1delta) | N-terminal GST | Negative | PV3665 |
| CSNK1E(CK1eps) | C-terminal His | Negative | PV3500 |
| CSNK1G1(CK1gamma1) | N-terminal GST | Negative | PV3825 |
| CSNK1G2 | C-terminal His | Negative | PV3499 |
| CSNK1G3 | N-terminal GST | Weak | PV3838 |
| CSNK2A1(CK2alpha1) | C-terminal His | Strong | PV3248 |
| CSNK2A2 | N-terminal GST | Negative | PV3624 |
| DAPK1 | | NT | PV3969 |
| DAPK2 | N-terminal GST | Negative | PV3614 |
| DAPK3(ZIPK) | N-terminal GST | Negative | PV3686 |
| DMPK | N-terminal GST | Negative | PV3784 |
| DYRK1A | N-terminal GST | Negative | PV3785 |
| DYRK3 | N-terminal GST | Negative | PV3837 |
| DYRK4 | | NT | PV3871 |
| GRK4 | N-terminal GST | Negative | PV3807 |
| GRK5 | N-terminal GST | Negative | PV3824 |
| GRK6 | N-terminal GST | Negative | PV3661 |
| GRK7 | N-terminal GST | Negative | PV3823 |
| GSK3A | C-terminal His | Weak | PV3270 |
| GSK3B | C-terminal His | Negative | PV3365 |
| HIPK4 | | NT | PV3852 |
| IKBKB | N-terminal GST | Weak | PV3836 |
| IRAK4 | N-terminal His | Strong | PV3362 |
| MAP2K1(MEK1) | N-terminal His | Negative | PV3303 |
| MAP2K1, mutant | C-terminal His | Negative | P3099 |
| MAP2K2(MEK2) | C-terminal His | Negative | PV3615 |
| MAP2K3(MEK3) | N-terminal GST | Negative | PV3662 |
| MAP2K6(MKK6) | N-terminal His | Weak | PV3318 |
| MAP2K6, mutant | N-terminal His | Negative | PV3293 |
| MAP3K11(MLK3) | N-terminal GST | Negative | PV3788 |
| MAP3K2(MEKK2) | N-terminal GST | Weak | PV3822 |
| MAP3K3(MEKK3) | | NT | PV3876 |
| MAP3K5(ASK1) | N-terminal GST | Weak | PV3809 |
| MAP3K9(MLK1) | N-terminal GST | Strong | PV3787 |
| MAP3K10(MLK2) | | NT | PV3877 |
| MAP4K4(HGK) | N-terminal GST | Strong | PV3687 |
| MAP4K5(KHS1) | N-terminal GST | Negative | PV3682 |
| MAPK1(ERK2) | N-terminal GST | Strong | PV3313 |
| MAPK11(p38B) | N-terminal His | Strong | PV3679 |
| MAPK12(p38gamma) | N-terminal His | Strong | PV3654 |
| MAPK13(p38delta) | N-terminal His | Weak | PV3656 |
| MAPK14(p38alpha) | N-terminal GST | Strong | PV3304 |
| MAPK3(ERK1) | N-terminal GST | Strong | PV3311 |
| MAPK8(JNK1) | N-terminal His | Negative | PV3319 |
| MAPK9(JNK2) | N-terminal His | NT | PV3620 |
| MAPKAPK2 | N-terminal His | Strong | PV3317 |
| MAPKAPK3 | N-terminal His | Strong | PV3299 |
| MAPKAPK5(PRAK) | N-terminal His | Strong | PV3301 |
| MARK2 | | NT | PV3878 |
| MARK4 | | NT | PV3851 |
| MINK1 | N-terminal GST | Weak | PV3810 |
| MLCK | N-terminal GST | Negative | PV3835 |
| MST4 | N-terminal GST | Negative | PV3690 |
| MYLK2 | N-terminal GST | Negative | PV3757 |
| NEK2 | C-terminal His | Strong | PV3360 |
| NEK3 | N-terminal GST | Negative | PV3821 |
| NEK6 | C-terminal His | Negative | PV3353 |
| NEK7 | N-terminal GST | Negative | PV3833 |
| PAK1 | N-terminal GST | Negative | PV3820 |
| PAK3 | N-terminal His | Weak | PV3789 |
| PAK4 | | NT | PV3845 |
| PAK6 | C-terminal His | Negative | PV3502 |
| PASK | | NT | PV3972 |
| PDK1 | N-terminal His | Weak | P3001(5 ug) |
| PHKG1 | N-terminal GST | Negative | PV3853 |
| PHKG2 | C-terminal His | Negative | PV3369 |
| PIM1 | C-terminal His | Strong | PV3503 |
| PIM2 | N-terminal GST | Weak | PV3649 |
| PKN1(PRK1) | N-terminal GST | Strong | PV3790 |
| PKN2(PRK2) | | NT | PV3879 |
| PLK1 | none | Negative | PV3501 |
| PLK3 | N-terminal GST | Negative | PV3812 |
| PRKACA(PKA) | N-terminal His | Negative | P2912 |
| PRKCA | none | Strong | P2232(5 ug), |
| | | | P2227(20 ug) |
| PRKCB1 | none | Strong | P2291, P2281 |
| PRKCB2 | none | Strong | P2254, P2251 |
| PRKCD | none | Strong | P2293, P2287 |
| PRKCE | none | Strong | P2292, P2282 |
| PRKCG | none | Strong | P2233, P2228 |
| PRKCH | N-terminal His | NT | P2633, P2634 |
| PRKCI | N-terminal His | Strong | PV3183, PV3186 |
| PRKCQ | C-terminal His | Strong | P2996 |
| PRKCZ | none | Strong | P2273, P2268 |
| PRKD1(PKD) | N-terminal GST | Weak | PV3791 |
| PRKD2(PKD2) | N-terminal GST | Strong | PV3758 |
| PRKD2 | N-terminal His | NT | PV3352 |
| PRKG2 | | NT | PV3973 |
| PRKCN(PKD3) | N-terminal GST | Weak | PV3692 |
| PRKX | N-terminal GST | Negative | PV3813 |
| RAF1 | N-terminal GST | Negative | PV3805 |
| RAFB | | NT | PV3848 |
| ROCK1 | N-terminal GST | Strong | PV3691 |
| ROCK2 | N-terminal GST | Strong | PV3759 |
| ROR2 | | NT | PV3861 |
| RPS6KA1(RSK1) | N-terminal His | Strong | PV3680 |
| RPS6KA2(RSK3) | N-terminal His | Negative | PV3846 |
| RPS6KA3(RSK2) | C-terminal His | Strong | PV3323 |
| RPS6KA4(MSK2) | N-terminal GST | Weak | PV3782 |
| RPS6KA5(MSK1) | N-terminal GST | Weak | PV3681 |
| RPS6KB1(p70S6K) | N-terminal GST | Negative | PV3815 |
| RPS6KB2(p70S6K2) | N-terminal GST | Negative | PV3831 |
| SGK | N-terminal GST | Strong | PV3818 |
| SGK2 | N-terminal GST | Weak | PV3858 |
| SGKL(SGK3) | N-terminal GST | Weak | PV3859 |
| SLK | N-terminal GST | Weak | PV3830 |
| SRPK2 | N-terminal GST | Strong | PV3829 |
| STK17A(DRAK1) | N-terminal GST | Negative | PV3783 |
| STK22B(TSSK2) | N-terminal His | Negative | PV3622 |
| STK22D(TSSK1) | C-terminal His | Strong | PV3505 |
| STK23(MSSK1) | | NT | PV3880 |
| STK31(SgK396) | | NT | PV3862 |
| STK4(MST1) | | NT | PV3854 |
| STK3(MST2) | N-terminal His | NT | PV3684 |
| STK24(MST3) | N-terminal GST | Weak | PV3650 |
| STK25(YSK1) | N-terminal GST | Strong | PV3657 |
| TAOK2(TAO1) | N-terminal GST | Strong | PV3760 |
| TAOK3(JIK) | N-terminal GST | Strong | PV3652 |
| TBK1 | N-terminal His | Strong | PV3504 |
| TTK | N-terminal GST | Negative | PV3792 |
| WEE1 | N-terminal GST | Negative | PV3817 |
| ZAK | | NT | PV3882 |

*NT indicates test was not performed

### Example 11

### Universal Substrate and Assay

As described above, another embodiment of the present invention is a “universal” substrate and assays using the same. This substrate comprises an amino acid sequence corresponding to at least a portion of MBP joined to at least one amino acid sequence different from that of MBP, such that both the MBP sequence and the non-MBP amino acid sequence have the ability to serve as the substrate for one or more kinases. It is preferred that the non-MBP amino acid sequence is a substrate for one or more kinases that do not phosphorylate MBP. By joining multiple non-MBP amino acid sequences to the MBP sequence, a universal substrate is provided that may serve as a substrate for each kinase in the human kinome.

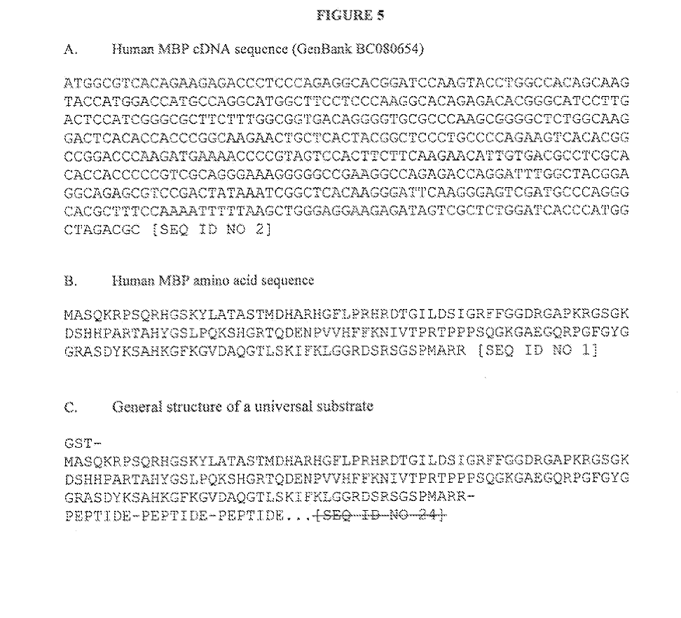

In this illustrative example, the starting material for producing the universal substrate is an expression vector (pDEST15) containing human MBP cDNA (FIG. 5) with GST fused at the N-terminus (hMBP-GST). An XhoI site is inserted at the 3′ end (C-terminus) of the hMBP-GST (using QuickChange™ from Stratagene). The vector is then treated with Xho1 in order to ligate into that site an oligonucleotide encoding a peptide and having XhoI complementary overhangs. After ligation, the original XhoI site is non-functional, but the ligated oligonucleotides contain a new XhoI site at the C-terminus. As such, this round of construction may be repeated to insert another peptide sequence. This is repeated until phosho-acceptor sites for every kinase are available on the MBP-peptide fusion sequence (i.e., the “universal substrate”). The general structure of a universal substrate is shown in FIG. 5(C):

An exemplary cloning strategy is shown below:

ENTR221 (MBP forward primer)

[SEQ ID NO:25]

GGGGACAAGTTTGTACAAAAAAGCAGGCACCATGGCGTCACAGAAGAGAC
CCTCC
ENTR221 (MBP reverse primer)

[SEQ ID NO:26]

GGGGACCACTTTGTACAAGAAAGCTGGGTTCTAGCGTCTAGCCATGGGTG
ATCC
hMBP XhoI construction for ligating peptide
fusions:
XhoI:

[SEQ ID NO:27]

5′: C′TCGAG (encodes Leu Val)

[SEQ ID NO:28]

3′: GAGCT′C

Quick Change Sequences for hMBP pDEST15:

OligoI:

[SEQ ID NO:29]

CTTTCGACCCAAGATGAGCTCCGCAGATCGGTACCC 22/39 GC
OligoII:

[SEQ ID NO:30]

GAAAGCTGGGTTCTACTCGAGGCGTCTAGCCATGGG 56%
hMBP pDEST15:

[SEQ ID NO:31]

GAAAGCTGGGTTCTAGCGTCTAGCCATGGG N = 30
Tm = 81.5 + 0.41(%GC) − 675/N = 81.5 + 0.41(56) −
675/30 = 81.5 + 22.96 − 22.5 = 82

Peptide Inserts/Cut Parent with XhoI/Ligate Peptides

| | |
|---|---|
| XhoI: | |
| 5′: C′TCGAG LeuVal | [SEQ ID NO:32] |
| 3′: GAGCT′C | [SEQ ID NO:33] |
| Cut w/XhoI: | |
| 5′: C TCGAG | [SEQ ID NO:34] |
| 3′: GAGCT C | [SEQ ID NO:35] |
| Insert: | |
| TCGACPEPTIDESEQUENCEC | [SEQ ID NO:36] |
| GPEPTIDESEQUENCEGAGCT | [SEQ ID NO:37] |
| Ligate: | |
| (no XhoI) XhoI | |
| CTCGACPEPTIDESEQUENCECTCGAG | [SEQ ID NO:38] |
| GAGCTGPEPTIDESEQUENCEGAGCTC | [SEQ ID NO:39] |
| Product: | |
| LeuAspPEPTIDESEQUENCELeuVal | [SEQ ID NO:40] |

### Peptide Inserts:

[SEQ ID NO:41]

EEEEYIQIVK Tyr 4

[SEQ ID NO:42]

5′: GAAGAAGAAGAATACATACAAATAGTAAAA

[SEQ ID NO:41]

3′: CTTCTTCTTCTTATGTATGTTTATCATTTT

[SEQ ID NO:44]

5′: TTTTACTATTTGTATGTATTCTTCTTCTTC

[SEQ ID NO:45]

EAEAIYAAPGDK Tyr 2

[SEQ ID NO:46]

5′: GAAGCAGAAGCAATATACGCAGCACCAGGAGACAAA

[SEQ ID NO:47]

3′: CTTCGTCTTCGTTATATGCGTCGTGGTCCTCTGTTT

[SEQ ID NO:48]

5′: TTTGTCTCCTGGTGCTGCGTATATTGCTTCTGCTTC
XhoI-T4T2F 72mer

[SEQ ID NO:49]

5′: TCGACGAAGAAGAAGAATACATACAAATAGTAAAAGAAGCAGAAGC
AATATACGCAGCACCAGGAGACAAAC
XhoI-T4T2R 72mer

[SEQ ID NO:50]

5′: TCGAGTTTGTCTCCTGGTGCTGCGTATATTGCTTCTGCTTCTTTTA
CTATTTGTATGTATTCTTCTTCTTCG

[SEQ ID NO:51]

EEEIYGVIEK Tyr 1

[SEQ ID NO:52]

5′: GAAGAAGAAATATACGGAGTAATAGAAAAA

[SEQ ID NO:53]

3′: CTTCTTCTTTATATGCCTCATTATCTTTTT

[SEQ ID NO:54]

5′: TTTTTCTATTACTCCGTATATTTCTTCTTC

[SEQ ID NO:55]

ALRRFSLGEK Ser/Thr 1

[SEQ ID NO:56]

5′: GCACTACGACGATTCTCACTAGGAGAAAAA

[SEQ ID NO:57]

3′: CGTGATGCTGCTAAGAGTGATCCTCTTTTT

[SEQ ID NO:58]

5′: TTTTTCTCCTAGTGAGAATCGTCGTAGTGC
XhoI-T1S1F 66mer

[SEQ ID NO:59]

5′: TCGACGAAGAAGAAATATACGGAGTAATAGAAAAAGCACTACGACG
ATTCTCACTAGGAGAAAAAC
XhoI-T1S1R 66mer

[SEQ ID NO:60]

5′: TCGAGTTTTTCTCCTAGTGAGAATCGTCGTAGTGCTTTTTCTATTA
CTCCGTATATTTCTTCTTCG

[SEQ ID NO:61]

KLNRVFSVAC Ser/Thr 4

[SEQ ID NO:62]

5′: AAACTAAACCGAGTATTCTCAGTAGCATGC

[SEQ ID NO:63]

3′: TTTGATTTGGCTCATAAGAGTCATCGTACG

[SEQ ID NO:64]

5′: GCATGCTACTGAGAATACTCGGTTTAGTTT

[SEQ ID NO:50]

RRRQFSLRRKAK Ser/Thr 7

[SEQ ID NO:65]

5′: CGACGACGACAATTCTCACTACGACGAAAAGCAAAA

[SEQ ID NO:66]

3′: GCTGCTGCTGTTAAGAGTGATGCTGCTTTTCGTTTT

[SEQ ID NO:67]

5′: TTTTGCTTTTCGTCGTAGTGAGAATTGTCGTCGTCG
XhoI-S4S7F 72mer

[SEQ ID NO:68]

5′: TCGACAAACTAAACCGAGTATTCTCAGTAGCATGCCGACGACGACA
ATTCTCACTACGACGAAAAGCAAAAC
XhoI-S4S7R 72mer

[SEQ ID NO:69]

5′: TCGAGTTTTGCTTTTCGTCGTAGTGAGAATTGTCGTCGTCGGCATG
CTACTGAGAATACTCGGTTTAGTTTG

The exemplary universal substrate so constructed has the following amino acid sequence:

[SEQ ID NO:70]

MASQKRPSQRHGSKYLATASTMDHARHGFLPRHRDTGILDSIGRFFGGDR
GAPKRGSGKDSHHPARTALHYGSLPQKSHGRTQDENPVVHFFKNIVTPRT
PPPSQGKGAEGQRPGFGYGGRASDYKSAHKGFKGVDAQGTLSKIFKLGGR
DSRSGSPMARR-LV-EEEEYIQIVK-LV-EAEAIYAAPGDK-LV-EEEIY
GVIEK-LV-ALRRFSLGEK-LV-KLNRVFSVAC-LV-RRRQFSLRRKAK

Additional peptide sequences may be added using the technique described above until a sufficient number of phosphor-acceptor sites are represented on the universal substrate. The linker (LV) may or may not included, as desired by the investigator.

The universal substrate may then be utilized in a kinase assay as described above in Example X. Briefly, the universal substrate is diluted to 1 mg/ml in PBS, applied to the slide surface, covered with a coverslip, and left overnight at 4° C. Slides are then washed 3 times with water and spun dry before printing. Kinases (Panvera/Invitrogen) are diluted in printing buffer (50 mM Tris pH 7.5, 25% glyercol, 0.05% Triton X-100, 2 mM DTT), deposited onto the slides using a GeneMachine OmniGrid100, and stored at −20° C.

Reactions are performed following removal of the slide from the freezer. Reaction buffer (20 mM HEPES pH 7.5, 4 mM MgCl2, 2 mM DTT, 20 uM ATP, 5% DMSO) is added with or without inhibitor, a coverslip applied, and the slide placed at 30° C. for the appropriate reaction time. The slide is washed with water to stop the reaction reaction (3 times) and spun dry. ProQ Diamond Microarray Stain (Invitrogen #P33706) is applied, covered with a coverslip, and the slide is incubated in the dark at room temperature for 30 minutes. The slide is destained and washed three times with water, and spun dry. Results are then acquired and analyzed using fluorometer (GenePix 4000B).

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled

### References Cited

All references cited herein are incorporated herein by reference in their entirety and for all purposes to the same extent as if each individual publication or patent or patent application was specifically and individually indicated to be incorporated by reference in its entirety for all purposes.

1. A method for detecting phosphorylation of myelin basic protein (MBP) by a kinase, the method comprising:
(a) incubating a tyrosine kinase and MBP, or a fragment or derivative thereof comprising at least 15 contiguous amino acids of MBP, or one or more conservative substitutions thereof, and comprising at least one phosphorylation site of MBP within the at least 15 contiguous amino acids, under conditions allowing for phosphorylation of the MBP or fragment or derivative thereof by the tyrosine kinase; and,
(b) detecting phosphorylation of the MBP, or the fragment or derivative thereof.

2. The method of claim 1, wherein the incubating step is done in the presence of a test molecule.

3. The method of claim 2, wherein the detecting step comprises detecting a decrease in the phosphorylation in the presence of the test molecule, thereby identifying the test molecule as an inhibitor of the kinase.

4. The method of claim 2, wherein the detecting step comprises detecting an increase in the phosphorylation in the presence of the test molecule, thereby identifying the test molecule as an activator of the kinase.

5. The method of claim 1, wherein step (b) comprises detecting phosphorylated tyrosines on the myelin basic protein or the fragment or derivative thereof.

6. The method of claim 1, wherein the determining step comprises contacting myelin basic protein, or a fragment or derivative thereof, with a binding partner that selectively binds to the phosphorylated or non-phosphorylated form of MBP or a fragment thereof.

7. The method of claim 1, wherein the tyrosine kinase is a tyrosine kinase of Table 2 or Table 6.

8. The method of claim 1, wherein the tyrosine kinase is selected from the group consisting of CSF1R, EPHA1, EPHA2, EPHA3, EPHA4, EPHA7, EPHA8, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, ABL1, ABL2(ARG), BLK, BMX, BTK, FGR, FYN, HCK, JAK3, LCK, LYNA, PTK6(BRK), SRC, and YES1.

9. The method of claim 1, wherein the tyrosine kinase is selected from the group consisting of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

10-12. (canceled)

13. The method of claim 1, wherein the tyrosine kinase is selected from two or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

14. The method of claim 1, wherein the tyrosine kinase is selected from five or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

15. The method of claim 1, wherein the tyrosine kinase is selected from ten or more of CSF1R, EPHA1, EPHA3, EPHA4, EPHB1, EPHB2, EPHB3, EPHB4, FGFR1, FGFR2, FGFR3, FGFR4, FLT1, FLT3, IGF1R, INSR, INSR, KDR, MERTK, MET, NTRK1, NTRK2, NTRK3, PDGFRA, PDGFRB, RET, ROS1, TYRO3, BMX, BTK, FYN, HCK, JAK3, LCK, PTK6(BRK), and SRC.

16. The method of claim 1, wherein the MBP or the fragment or derivative thereof, is MBP or a fragment thereof comprising at least 15 contiguous amino acids of MBP.

17. The method of claim 1, wherein the MBP or the fragment or derivative thereof, is full length MBP.

18. The method of claim 1, wherein the MBP or the fragment or derivative thereof, is full length human MBP or a fragment thereof comprising at least 15 contiguous amino acids of human MBP.

19. The method of claim 1, wherein the MBP or the fragment or derivative thereof, is full length bovine MBP or a fragment thereof comprising at least 15 contiguous amino acids of bovine MBP.

20. The method of claim 1, wherein the MBP or fragment or derivative thereof, at the start of the incubating, is not phosphorylated.

21. The method of claim 1, further comprising isolating the MBP or the fragment or derivative thereof from a prokaryotic host cell.

22. The method of claim 1, wherein at least one of the tyrosine kinase and the MBP or the fragment or derivative thereof, are immobilized on the surface of a solid support.

23. The method of claim 1, wherein both the tyrosine kinase and the MBP or the fragment or derivative thereof, are immobilized on the surface of a solid support.

24. (canceled)

25. The method of claim 23, wherein the MBP or the fragment or derivative thereof, is coated onto the surface of the solid support and the kinase is deposited onto the surface of the solid support.

26. (canceled)

27. The method of claim 23, wherein a kinase substrate other than MBP or a fragment or derivative thereof, is coated onto the surface of the solid support along with MBP or a fragment or derivative thereof.

28. The method of claim 23, wherein a plurality of kinases are immobilized on the solid support, wherein at least one of the plurality of kinases is other than a tyrosine kinase.

29-31. (canceled)

32. The method of claim 28, wherein the plurality of different kinases comprises a tyrosine kinase and a serine/threonine kinase.

33. The method of claim 32, wherein the detecting comprises detecting phosphorylation of MBP, or the fragment or derivative thereof, by the tyrosine kinase and/or by the serine/threonine kinase, wherein both the tyrosine kinase and the serine/threonine kinase phosphorylate MBP, or the fragment or derivative thereof.

34. The method of claim 28, wherein a plurality of different substrates are immobilized on the solid support.

35-40. (canceled)

41. The method of claim 1, wherein the MBP or the fragment or derivative thereof, is a first amino acid sequence of a recombinant fusion protein further comprising a second amino acid sequence comprising a kinase substrate other than MBP or a fragment or derivative thereof.

42. The method of claim 41, wherein the second amino acid sequence is a substrate for a kinase that does not phosphorylate MBP.

43. The method of claim 41, wherein the recombinant fusion protein comprises additional amino acid sequences that are kinase substrate such that the recombinant fusion protein is phosphorylated by at least 100 kinases.

44-88. (canceled)


SEQUENCE LISTING (59 sequences)

SEQ ID NO: 1 - protein, 160 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 2 - DNA, 480 bp, Homo sapiens
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgc

SEQ ID NO: 3 - protein, 10 aa, Artificial Sequence
  Description of Artificial Sequence Synthetic kinase substrate peptide
ALRRFSLGEK

SEQ ID NO: 4 - protein, 13 aa, Artificial Sequence
  Description of Artificial Sequence Synthetic kinase substrate peptide
RGGLFSTTPGGTK

SEQ ID NO: 5 - protein, 11 aa, Artificial Sequence
  Description of Artificial Sequence Synthetic kinase substrate peptide
VAPFSPGGRAK

SEQ ID NO: 6 - protein, 10 aa, Artificial Sequence
  Description of Artificial Sequence Synthetic kinase substrate peptide
KLNRVFSVAC

SEQ ID NO: 7 - protein, 10 aa, Artificial Sequence
  Description of Artificial Sequence Synthetic kinase substrate peptide
GDQDYLSLDK

SEQ ID NO: 8 - protein, 10 aa, Artificial Sequence
  Description of Artificial Sequence Synthetic kinase substrate peptide
ARPRAFSVGK

SEQ ID NO: 9 - protein, 12 aa, Artificial Sequence
  Description of Artificial Sequence Synthetic kinase substrate peptide
RRRQFSLRRKAK

SEQ ID NO: 10 - protein, 12 aa, Artificial Sequence
  Description of Artificial Sequence Synthetic kinase substrate peptide
RPRTFSSLAEGK

SEQ ID NO: 11 - protein, 13 aa, Artificial Sequence
  Description of Artificial Sequence Synthetic kinase substrate peptide
PRPFSVPPPSPDK

SEQ ID NO: 12 - protein, 14 aa, Artificial Sequence
  Description of Artificial Sequence Synthetic kinase substrate peptide
KKKALSRQFSVAAK

SEQ ID NO: 13 - protein, 9 aa, Artificial Sequence
  Description of Artificial Sequence Synthetic kinase substrate peptide
ESFSSSEEK

SEQ ID NO: 14 - protein, 16 aa, Artificial Sequence
  Description of Artificial Sequence Synthetic kinase substrate peptide
VLAKSFGSPNRARKKK

SEQ ID NO: 15 - protein, 12 aa, Artificial Sequence
  Description of Artificial Sequence Synthetic kinase substrate peptide
KKRPQRRYSNVL

SEQ ID NO: 16 - protein, 10 aa, Artificial Sequence
  Description of Artificial Sequence Synthetic kinase substrate peptide
RRRLSFAEPG

SEQ ID NO: 17 - protein, 16 aa, Artificial Sequence
  Description of Artificial Sequence Synthetic kinase substrate peptide
LVEPFTPSGEAPNQKK

SEQ ID NO: 18 - protein, 13 aa, Artificial Sequence
  Description of Artificial Sequence Synthetic kinase substrate peptide
EVIEASFAEQEAK

SEQ ID NO: 19 - protein, 10 aa, Artificial Sequence
  Description of Artificial Sequence Synthetic kinase substrate peptide
EEEIYGVIEK

SEQ ID NO: 20 - protein, 12 aa, Artificial Sequence
  Description of Artificial Sequence Synthetic kinase substrate peptide
EAEAIYAAPGDK

SEQ ID NO: 21 - protein, 11 aa, Artificial Sequence
  Description of Artificial Sequence Synthetic kinase substrate peptide
GVLTGYVARRK

SEQ ID NO: 22 - protein, 10 aa, Artificial Sequence
  Description of Artificial Sequence Synthetic kinase substrate peptide
EEEEYIQIVK

SEQ ID NO: 23 - protein, 11 aa, Artificial Sequence
  Description of Artificial Sequence Synthetic kinase substrate peptide
AAEEIYAARRG

SEQ ID NO: 24 - DNA, 55 bp, Artificial Sequence
  Description of Artificial Sequence Synthetic primer
ggggacaagtttgtacaaaaaagcaggcaccatggcgtcacagaagagaccctcc

SEQ ID NO: 25 - DNA, 54 bp, Artificial Sequence
  Description of Artificial Sequence Synthetic primer
ggggaccactttgtacaagaaagctgggttctagcgtctagccatgggtgatcc

SEQ ID NO: 26 - DNA, 36 bp, Homo sapiens
ctttcgacccaagatgagctccgcagatcggtaccc

SEQ ID NO: 27 - DNA, 36 bp, Homo sapiens
gaaagctgggttctactcgaggcgtctagccatggg

SEQ ID NO: 28 - DNA, 30 bp, Homo sapiens
gaaagctgggttctagcgtctagccatggg

SEQ ID NO: 29 - protein, 10 aa, Artificial Sequence
  Description of Artificial Sequence Synthetic peptide insert
EEEEYIQIVK

SEQ ID NO: 30 - DNA, 30 bp, Artificial Sequence
  Description of Artificial Sequence Synthetic oligonucleotide
gaagaagaagaatacatacaaatagtaaaa

SEQ ID NO: 31 - DNA, 30 bp, Artificial Sequence
  Description of Artificial Sequence Synthetic oligonucleotide
cttcttcttcttatgtatgtttatcatttt

SEQ ID NO: 32 - DNA, 30 bp, Artificial Sequence
  Description of Artificial Sequence Synthetic oligonucleotide
ttttactatttgtatgtattcttcttcttc

SEQ ID NO: 33 - protein, 12 aa, Artificial Sequence
  Description of Artificial Sequence Synthetic peptide insert
EAEAIYAAPGDK

SEQ ID NO: 34 - DNA, 36 bp, Artificial Sequence
  Description of Artificial Sequence Synthetic oligonucleotide
gaagcagaagcaatatacgcagcaccaggagacaaa

SEQ ID NO: 35 - DNA, 36 bp, Artificial Sequence
  Description of Artificial Sequence Synthetic oligonucleotide
cttcgtcttcgttatatgcgtcgtggtcctctgttt

SEQ ID NO: 36 - DNA, 36 bp, Artificial Sequence
  Description of Artificial Sequence Synthetic oligonucleotide
tttgtctcctggtgctgcgtatattgcttctgcttc

SEQ ID NO: 37 - DNA, 72 bp, Artificial Sequence
  Description of Artificial Sequence Synthetic Xhol-T4T2F oligonucleotide
tcgacgaagaagaagaatacatacaaatagtaaaagaagcagaagcaatatacgcagcaccaggagacaaac

SEQ ID NO: 38 - protein, 72 aa, Artificial Sequence
  Description of Artificial Sequence Synthetic Xhol-T4T2R polypeptide
TCGAGTTTGTCTCCTGGTGCTGCGTATATTGCTTCTGCTTCTTTTACTATTTGTATGTATTCTTCTTCTTCG

SEQ ID NO: 39 - protein, 10 aa, Artificial Sequence
  Description of Artificial Sequence Synthetic Xhol-T4T2R peptide
EEEIYGVIEK

SEQ ID NO: 40 - DNA, 30 bp, Artificial Sequence
  Description of Artificial Sequence Synthetic Xhol-T4T2R oligonucleotide
gaagaagaaatatacggagtaatagaaaaa

SEQ ID NO: 41 - DNA, 30 bp, Artificial Sequence
  Description of Artificial Sequence Synthetic Xhol-T4T2R oligonucleotide
cttcttctttatatgcctcattatcttttt

SEQ ID NO: 42 - DNA, 30 bp, Artificial Sequence
  Description of Artificial Sequence Synthetic Xhol-T4T2R oligonucleotide
tttttctattactccgtatatttcttcttc

SEQ ID NO: 43 - protein, 10 aa, Artificial Sequence
  Description of Artificial Sequence Synthetic Xhol-T4T2R peptide
ALRRFSLGEK

SEQ ID NO: 44 - DNA, 30 bp, Artificial Sequence
  Description of Artificial Sequence Synthetic Xhol-T4T2R oligonucleotide
gcactacgacgattctcactaggagaaaaa

SEQ ID NO: 45 - DNA, 30 bp, Artificial Sequence
  Description of Artificial Sequence Synthetic Xhol-T4T2R oligonucleotide
cgtgatgctgctaagagtgatcctcttttt

SEQ ID NO: 46 - DNA, 30 bp, Artificial Sequence
  Description of Artificial Sequence Synthetic Xhol-T4T2R oligonucleotide
tttttctcctagtgagaatcgtcgtagtgc

SEQ ID NO: 47 - DNA, 66 bp, Artificial Sequence
  Description of Artificial Sequence Synthetic Xhol-T1S1F oligonucleotide
tcgacgaagaagaaatatacggagtaatagaaaaagcactacgacgattctcactaggagaaaaac

SEQ ID NO: 48 - DNA, 66 bp, Artificial Sequence
  Description of Artificial Sequence Synthetic Xhol-T1S1R oligonucleotide
tcgagtttttctcctagtgagaatcgtcgtagtgctttttctattactccgtatatttcttcttcg

SEQ ID NO: 49 - protein, 10 aa, Artificial Sequence
  Description of Artificial Sequence Synthetic XhoI-T1S1R peptide
KLNRVFSVAC

SEQ ID NO: 50 - DNA, 30 bp, Artificial Sequence
  Description of Artificial Sequence Synthetic XhoI-T1S1R oligonucleotide
aaactaaaccgagtattctcagtagcatgc

SEQ ID NO: 51 - DNA, 30 bp, Artificial Sequence
  Description of Artificial Sequence Synthetic XhoI-T1S1R oligonucleotide
tttgatttggctcataagagtcatcgtacg

SEQ ID NO: 52 - DNA, 30 bp, Artificial Sequence
  Description of Artificial Sequence Synthetic XhoI-T1S1R oligonucleotide
gcatgctactgagaatactcggtttagttt

SEQ ID NO: 53 - protein, 12 aa, Artificial Sequence
  Description of Artificial Sequence Synthetic XhoI-T1S1R peptide
RRRQFSLRRKAK

SEQ ID NO: 54 - DNA, 36 bp, Artificial Sequence
  Description of Artificial Sequence Synthetic XhoI-T1S1RR oligonucleotide
cgacgacgacaattctcactacgacgaaaagcaaaa

SEQ ID NO: 55 - DNA, 36 bp, Artificial Sequence
  Description of Artificial Sequence Synthetic XhoI-T1S1R oligonucleotide
gctgctgctgttaagagtgatgctgcttttcgtttt

SEQ ID NO: 56 - DNA, 36 bp, Artificial Sequence
  Description of Artificial Sequence Synthetic XhoI-T1S1R oligonucleotide
ttttgcttttcgtcgtagtgagaattgtcgtcgtcg

SEQ ID NO: 57 - DNA, 72 bp, Artificial Sequence
  Description of Artificial Sequence Synthetic XhoI-S4S7F oligonucleotide
tcgacaaactaaaccgagtattctcagtagcatgccgacgacgacaattctcactacgacgaaaagcaaaac

SEQ ID NO: 58 - DNA, 72 bp, Artificial Sequence
  Description of Artificial Sequence Synthetic XhoI-S4S7R oligonucleotide
tcgagttttgcttttcgtcgtagtgagaattgtcgtcgtcggcatgctactgagaatactcggtttagtttg

SEQ ID NO: 59 - protein, 236 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARRLVEEEEYIQIVKLVEAEAIYAAPGDKLVEEEIYGVIEKLVALRRFSLGEKLVKLNRVFSVACLVRRRQFSLRRKAK